SEQUENCE LISTING (49 sequences)

SEQ ID NO: 1 - DNA, 363 bp, synthetic construct
  misc_feature 1..363: note = Synthetic
  source 1..363: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaaggacttgagtggatgggatggatcagcccttacaatgttaacacaaactatgcacagaagctccagggcagagtcaccatgaccacagacacatccacgaacacagcctacatggagctgaggagcctgagatctgacgactcggccgtgtattactgttcgagagctgggatgccagctcgtcgctacggtatggacgtctggggccaagggaccacggtcaccgtctcctca

SEQ ID NO: 2 - protein, 121 aa, synthetic construct
  REGION 1..121: note = Synthetic
QVQLVQSGAEVKKPGASVKVSCKASGYTFTSYGISWVRQAPGQGLEWMGWISPYNVNTNYAQKLQGRVTMTTDTSTNTAYMELRSLRSDDSAVYYCSRAGMPARRYGMDVWGQGTTVTVSS

SEQ ID NO: 3 - DNA, 24 bp, synthetic construct
  misc_feature 1..24: note = Synthetic
  source 1..24: mol_type = other DNA
ggttacacctttaccagctatggt

SEQ ID NO: 4 - protein, 8 aa, synthetic construct
  REGION 1..8: note = Synthetic
GYTFTSYG

SEQ ID NO: 5 - DNA, 24 bp, synthetic construct
  misc_feature 1..24: note = Synthetic
  source 1..24: mol_type = other DNA
atcagcccttacaatgttaacaca

SEQ ID NO: 6 - protein, 8 aa, synthetic construct
  REGION 1..8: note = Synthetic
ISPYNVNT

SEQ ID NO: 7 - DNA, 42 bp, synthetic construct
  misc_feature 1..42: note = Synthetic
  source 1..42: mol_type = other DNA
tcgagagctgggatgccagctcgtcgctacggtatggacgtc

SEQ ID NO: 8 - protein, 14 aa, synthetic construct
  REGION 1..14: note = Synthetic
SRAGMPARRYGMDV

SEQ ID NO: 9 - DNA, 366 bp, synthetic construct
  misc_feature 1..366: note = Synthetic
  source 1..366: mol_type = other DNA
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggatcacccactacaacccctccctcaagagtcgagtcaccatatcagtagacacgtccaagatccagttctccctgaagctgagttctgtgaccgctgcggacacggccgtgtattactgtgcgagatggggggttcggagggactactactactacggtatggacgtctggggccaagggaccacggtcaccgtctcctca

SEQ ID NO: 10 - protein, 122 aa, synthetic construct
  REGION 1..122: note = Synthetic
QVQLQESGPGLVKPSETLSLTCTVSGGSISSYYWSWIRQPPGKGLEWIGYIYYSGITHYNPSLKSRVTISVDTSKIQFSLKLSSVTAADTAVYYCARWGVRRDYYYYGMDVWGQGTTVTVSS

SEQ ID NO: 11 - DNA, 24 bp, synthetic construct
  misc_feature 1..24: note = Synthetic
  source 1..24: mol_type = other DNA
ggtggctccatcagtagttactac

SEQ ID NO: 12 - protein, 8 aa, synthetic construct
  REGION 1..8: note = Synthetic
GGSISSYY

SEQ ID NO: 13 - DNA, 21 bp, synthetic construct
  misc_feature 1..21: note = Synthetic
  source 1..21: mol_type = other DNA
atctattacagtgggatcacc

SEQ ID NO: 14 - protein, 7 aa, synthetic construct
  REGION 1..7: note = Synthetic
IYYSGIT

SEQ ID NO: 15 - DNA, 48 bp, synthetic construct
  misc_feature 1..48: note = Synthetic
  source 1..48: mol_type = other DNA
gcgagatggggggttcggagggactactactactacggtatggacgtc

SEQ ID NO: 16 - protein, 16 aa, synthetic construct
  REGION 1..16: note = Synthetic
ARWGVRRDYYYYGMDV

SEQ ID NO: 17 - DNA, 324 bp, synthetic construct
  misc_feature 1..324: note = Synthetic
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 18 - protein, 108 aa, synthetic construct
  REGION 1..108: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 19 - DNA, 21 bp, synthetic construct
  misc_feature 1..21: note = Synthetic
  source 1..21: mol_type = other DNA
cagtcagtctctagctcttat

SEQ ID NO: 20 - protein, 7 aa, synthetic construct
  REGION 1..7: note = Synthetic
QSVSSSY

SEQ ID NO: 21-22 - skipped (2 entries)

SEQ ID NO: 23 - DNA, 27 bp, synthetic construct
  misc_feature 1..27: note = Synthetic
  source 1..27: mol_type = other DNA
caacagtacggaagcagcccgtggacg

SEQ ID NO: 24 - protein, 9 aa, synthetic construct
  REGION 1..9: note = Synthetic
QQYGSSPWT

SEQ ID NO: 25 - DNA, 1344 bp, synthetic construct
  misc_feature 1..1344: note = Synthetic
  source 1..1344: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaaggacttgagtggatgggatggatcagcccttacaatgttaacacaaactatgcacagaagctccagggcagagtcaccatgaccacagacacatccacgaacacagcctacatggagctgaggagcctgagatctgacgactcggccgtgtattactgttcgagagctgggatgccagctcgtcgctacggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 26 - protein, 447 aa, synthetic construct
  REGION 1..447: note = Synthetic
QVQLVQSGAEVKKPGASVKVSCKASGYTFTSYGISWVRQAPGQGLEWMGWISPYNVNTNYAQKLQGRVTMTTDTSTNTAYMELRSLRSDDSAVYYCSRAGMPARRYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - DNA, 1347 bp, synthetic construct
  misc_feature 1..1347: note = Synthetic
  source 1..1347: mol_type = other DNA
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggatcacccactacaacccctccctcaagagtcgagtcaccatatcagtagacacgtccaagatccagttctccctgaagctgagttctgtgaccgctgcggacacggccgtgtattactgtgcgagatggggggttcggagggactactactactacggtatggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 28 - protein, 448 aa, synthetic construct
  REGION 1..448: note = Synthetic
QVQLQESGPGLVKPSETLSLTCTVSGGSISSYYWSWIRQPPGKGLEWIGYIYYSGITHYNPSLKSRVTISVDTSKIQFSLKLSSVTAADTAVYYCARWGVRRDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 29 - DNA, 648 bp, synthetic construct
  misc_feature 1..648: note = Synthetic
  source 1..648: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 30 - protein, 215 aa, synthetic construct
  REGION 1..215: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 360 bp, synthetic construct
  misc_feature 1..360: note = Synthetic
  source 1..360: mol_type = other DNA
caggtgcagctgcaggagtcgggcccaggactggttcagccttcggagaccctgtccctcacctgcactgtctctggtggctccatcagtaattactactggagctggatccggcaggccccaggaaagggactggaatggattggatatgtctatcacagtgggagcaccaattccaagtccgccctcaagagtcgagtcaccatatcagttgacacgtccaagaatcagttctcccttaaactgcggactgtgaccgccgcagacacggccgtatatttctgtgcgagacatatcccagcccgttattactatggcatggacgtctggggccaagggaccacggtcaccgtctcctca

SEQ ID NO: 32 - protein, 120 aa, synthetic construct
  REGION 1..120: note = Synthetic
QVQLQESGPGLVQPSETLSLTCTVSGGSISNYYWSWIRQAPGKGLEWIGYVYHSGSTNSKSALKSRVTISVDTSKNQFSLKLRTVTAADTAVYFCARHIPARYYYGMDVWGQGTTVTVSS

SEQ ID NO: 33 - DNA, 24 bp, synthetic construct
  misc_feature 1..24: note = Synthetic
  source 1..24: mol_type = other DNA
ggtggctccatcagtaattactac

SEQ ID NO: 34 - protein, 8 aa, synthetic construct
  REGION 1..8: note = Synthetic
GGSISNYY

SEQ ID NO: 35 - DNA, 21 bp, synthetic construct
  misc_feature 1..21: note = Synthetic
  source 1..21: mol_type = other DNA
gtctatcacagtgggagcacc

SEQ ID NO: 36 - protein, 7 aa, synthetic construct
  REGION 1..7: note = Synthetic
VYHSGST

SEQ ID NO: 37 - DNA, 42 bp, synthetic construct
  misc_feature 1..42: note = Synthetic
  source 1..42: mol_type = other DNA
gcgagacatatcccagcccgttattactatggcatggacgtc

SEQ ID NO: 38 - protein, 14 aa, synthetic construct
  REGION 1..14: note = Synthetic
ARHIPARYYYGMDV

SEQ ID NO: 39 - DNA, 1341 bp, synthetic construct
  misc_feature 1..1341: note = Synthetic
  source 1..1341: mol_type = other DNA
caggtgcagctgcaggagtcgggcccaggactggttcagccttcggagaccctgtccctcacctgcactgtctctggtggctccatcagtaattactactggagctggatccggcaggccccaggaaagggactggaatggattggatatgtctatcacagtgggagcaccaattccaagtccgccctcaagagtcgagtcaccatatcagttgacacgtccaagaatcagttctcccttaaactgcggactgtgaccgccgcagacacggccgtatatttctgtgcgagacatatcccagcccgttattactatggcatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 40 - protein, 446 aa, synthetic construct
  REGION 1..446: note = Synthetic
QVQLQESGPGLVQPSETLSLTCTVSGGSISNYYWSWIRQAPGKGLEWIGYVYHSGSTNSKSALKSRVTISVDTSKNQFSLKLRTVTAADTAVYFCARHIPARYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - DNA, 363 bp, synthetic construct
  misc_feature 1..363: note = Synthetic
  source 1..363: mol_type = other DNA
caggtgcagctgcaggagtcgggcccaggactggtgaagccttcggagaccctgtccctcacctgcactgtctctggtggctccatcagtagttactactggagttggatccggcagcccccagggaagggactggaatggattgggtctatttattacagtgggggcaccaattacaacccctccctcaagagtcgagtcaccatatcattagacacgtccaagaaccagttctccctgaagctgagatctgggaccgctgcggacacggccgtttatttttgtaccagaaataactggttctacgggagggtggggggccttgactactggggccagggaaccctggtcaccgtctcctca

SEQ ID NO: 42 - protein, 121 aa, synthetic construct
  REGION 1..121: note = Synthetic
QVQLQESGPGLVKPSETLSLTCTVSGGSISSYYWSWIRQPPGKGLEWIGSIYYSGGTNYNPSLKSRVTISLDTSKNQFSLKLRSGTAADTAVYFCTRNNWFYGRVGGLDYWGQGTLVTVSS

SEQ ID NO: 43 - DNA, 21 bp, synthetic construct
  misc_feature 1..21: note = Synthetic
  source 1..21: mol_type = other DNA
atttattacagtgggggcacc

SEQ ID NO: 44 - protein, 7 aa, synthetic construct
  REGION 1..7: note = Synthetic
IYYSGGT

SEQ ID NO: 45 - DNA, 45 bp, synthetic construct
  misc_feature 1..45: note = Synthetic
  source 1..45: mol_type = other DNA
accagaaataactggttctacgggagggtggggggccttgactac

SEQ ID NO: 46 - protein, 15 aa, synthetic construct
  REGION 1..15: note = Synthetic
TRNNWFYGRVGGLDY

SEQ ID NO: 47 - DNA, 1344 bp, synthetic construct
  misc_feature 1..1344: note = Synthetic
  source 1..1344: mol_type = other DNA
caggtgcagctgcaggagtcgggcccaggactggtgaagccttcggagaccctgtccctcacctgcactgtctctggtggctccatcagtagttactactggagttggatccggcagcccccagggaagggactggaatggattgggtctatttattacagtgggggcaccaattacaacccctccctcaagagtcgagtcaccatatcattagacacgtccaagaaccagttctccctgaagctgagatctgggaccgctgcggacacggccgtttatttttgtaccagaaataactggttctacgggagggtggggggccttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 48 - protein, 447 aa, synthetic construct
  REGION 1..447: note = Synthetic
QVQLQESGPGLVKPSETLSLTCTVSGGSISSYYWSWIRQPPGKGLEWIGSIYYSGGTNYNPSLKSRVTISLDTSKNQFSLKLRSGTAADTAVYFCTRNNWFYGRVGGLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 49 - protein, 367 aa, synthetic construct
  REGION 1..367: note = Synthetic
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

(19) United States
(12) Patent Application Publication
(10) Pub. No.: US 2026/0014252 A1
(43) Pub. Date: Jan. 15, 2026

(54) GPRC5D x CD28 Bispecific Antibodies and Methods of Use Thereof

(71) Applicant: Regeneron Pharmaceuticals, Inc., Tarrytown, NY (US)

(72) Inventors: David DiLillo, New York, NY (US); Kara Olson, White Plains, NY (US); Erica Ullman, Yorktown Heights, NY (US)

(21) Appl. No.: 19/265,769

(22) Filed: Jul. 10, 2025

Related U.S. Application Data
(60) Provisional application No. 63/670,016, filed on Jul. 11, 2024.

(51) Int. Cl. A61K 39/395; A61P 35/00; C07K 16/28

(52) U.S. Cl. CPC A61K 39/3955; A61P 35/00; C07K 16/2818; C07K 16/2866; C07K 2317/31; C07K 2317/92

(57) ABSTRACT

The present disclosure provides bispecific antigen-binding molecules comprising a first antigen-binding domain that binds specifically to GPRC5D and a second antigen-binding domain that binds specifically to CD28. In certain embodiments, the antigen-binding molecules are bispecific antibodies or antigen-binding fragments thereof that comprise a first antigen-binding domain that binds specifically to GPRC5D and a second antigen-binding domain that binds specifically to CD28. In certain embodiments, the bispecific antibodies are useful for treating a cancer (e.g., multiple myeloma).

### CROSS-REFERENCE TO RELATED APPLICATIONS

This application claims the benefit under 35 USC § 119 (e) of U.S. Provisional Application No. 63/670,016, filed Jul. 11, 2024, which is incorporated herein by reference in its entirety for all purposes.

### REFERENCE TO A SEQUENCE LISTING

This application incorporates by reference a computer readable Sequence Listing in ST.26 XML format, titled 11586US01_Sequence, created on Jul. 1, 2025 and containing 70,579 bytes.

### FIELD OF THE INVENTION

The present disclosure relates to antibodies, and antigen-binding fragments thereof, which are specific for G protein-coupled receptor, class C group 5 member D (GPRC5D), and methods of use thereof. The present disclosure includes bispecific antigen-binding molecules (e.g., bispecific antibodies) that bind GPRC5D and CD28, and methods of use thereof.

### BACKGROUND

The orphan G protein-coupled receptor, class C group 5 member D (GPRC5D) has been shown to be expressed on malignant bone marrow plasma cells, whereas normal tissue expression is limited to the hair follicle (Smith et al., Sci Transl Med., 11(485):1-26, 2019). GPRC5D is also reportedly expressed in differentiating cells that produce hard keratin, including cortical cells of the hair shaft, the keratogenous zone of the nail, and in a central region of the filiform papillae of the tongue (Inoue et al., J Invest Dermatol., 122(3):565-573, 2004). GPRC5D is an orphan receptor with no known ligand or function in human cancer. The GPRC5D encoding gene, which is mapped on chromosome 12p13.3, contains three exons and spans about 9.6 kb (Brauner-Osborne, et al., Biochim Biophys Acta., 1518(3):237-48, 2001). The large first exon encodes the seven-transmembrane domain. The biology of GPRC5D is however largely unknown.

CD28 is a type I transmembrane protein, which has a single extracellular Ig-V-like domain assembled as a homodimer and which is expressed on the surface of T cells. CD28 is the receptor for the CD80 (B7.1) and CD86 (B7.2) proteins and is activated by CD80 or CD86 expressed on antigen-presenting cells (APCs). The binding of CD28 to CD80 or CD86 provides co-stimulatory signals important for T cell activation and survival. T cell stimulation through CD28, in addition to the T-cell receptor (TCR), provides a potent signal for the production of various interleukins. CD28 also potentiates cellular signals such as pathways controlled by the NFκB transcription factor after TCR activation. The CD28 co-signal is important for effective T-cell activation such as T cell differentiation, proliferation, cytokine release and cell-death.

Multiple myeloma is a malignant neoplasm of plasma cells in the bone marrow associated with an overproduction of monoclonal (M)-protein often causing characteristic osteolytic lesions, anemia, renal failure, and hypercalcemia. Monoclonal gammopathy of unknown significance (MGUS) and smoldering multiple myeloma (SMM) are asymptomatic plasma cell disorders that result in increasing risks of progression to symptomatic multiple myeloma. Antigen-binding molecules that target GPRC5D, including bispecific antigen-binding molecules that bind both GPRC5D and CD28 would be useful in therapeutic settings in which specific targeting and T cell-mediated killing of cells that express GPRC5D is desired.

### SUMMARY OF THE DISCLOSURE

In one aspect, the present disclosure provides an isolated bispecific antigen-binding molecule comprising: (a) a first antigen-binding domain that specifically binds to human G protein-coupled receptor, class C group 5 member D (GPRC5D); and (b) a second antigen-binding domain that specifically binds human CD28. In some cases, the first antigen-binding domain binds to human GPRC5D within residues comprising an extracellular loop (ECL) 2 and ECL3 of GPRC5D.

In some embodiments, the first antigen-binding domain comprises a heavy chain variable region (HCVR) comprising three heavy chain complementarity determining regions (CDRs), HCDR1, HCDR2 and HCDR3, and a light chain variable region (LCVR) comprising three light chain complementarity determining regions (CDRs), LCDR1, LCDR2 and LCDR3, wherein: (a) HCDR1, HCDR2 and HCDR3 are the CDRs contained within a HCVR comprising the amino acid sequence of SEQ ID NO: 2; or (b) HCDR1, HCDR2 and HCDR3 are the CDRs contained within a HCVR comprising the amino acid sequence of SEQ ID NO: 32; or (c) HCDR1, HCDR2 and HCDR3 are the CDRs contained within a HCVR comprising the amino acid sequence of SEQ ID NO: 42. In some embodiments, LCDR1, LCDR2 and LCDR3 of the first antigen-binding domain are the CDRs contained within a LCVR comprising the amino acid sequence of SEQ ID NO: 18. In some embodiments, the second antigen-binding domain comprises a heavy chain variable region (HCVR) comprising three heavy chain complementarity determining regions (CDRs), HCDR1, HCDR2 and HCDR3, and a light chain variable region (LCVR) comprising three light chain complementarity determining regions (CDRs), LCDR1, LCDR2 and LCDR3, wherein HCDR1, HCDR2 and HCDR3 are the CDRs contained within a HCVR comprising the amino acid sequence of SEQ ID NO: 10. In some embodiments, the LCDR1, LCDR2 and LCDR3 of the second antigen-binding domain are the CDRs contained within a LCVR comprising the amino acid sequence of SEQ ID NO: 18.

In some embodiments, the first antigen-binding domain of the bispecific antigen-binding molecule comprises a HCVR comprising HCDR1, HCDR2 and HCDR3 comprising the amino acid sequences of SEQ ID NOs: 4, 6 and 8, respectively, and a LCVR comprising LCDR1, LCDR2 and LCDR3 comprising the amino acid sequences of SEQ ID NOs: 20, 22 and 24, respectively, and the second antigen-binding domain of the bispecific antigen-binding molecule comprises a HCVR comprising HCDR1, HCDR2 and HCDR3 comprising the amino acid sequences of SEQ ID NOs: 10, 12 and 14, respectively, and a LCVR comprising LCDR1, LCDR2 and LCDR3 comprising the amino acid sequences of SEQ ID NOs: 20, 22 and 24, respectively. In some cases, the first antigen-binding domain comprises a HCVR comprising the amino acid sequence of SEQ ID NO: 2 and a LCVR comprising the amino acid sequence of SEQ ID NO: 18, and the second antigen-binding domain comprises a HCVR comprising the amino acid sequence of SEQ ID NO: 10 and a LCVR comprising the amino acid sequence of SEQ ID NO: 18. In some embodiments, the bispecific antigen-binding molecule is a bispecific antibody comprising: a first heavy chain and a paired light chain interconnected by disulfide bonds, wherein the first heavy chain comprises a HCVR and a heavy chain constant region comprising CH1, CH2, and CH3 domains, and the paired light chain comprises a LCVR and a light chain constant region, wherein the first heavy chain and paired light chain comprise the first antigen-binding domain; and a second heavy chain and a paired light chain interconnected by disulfide bonds, wherein the second heavy chain comprises a HCVR and a heavy chain constant region comprising CH1, CH2, and CH3 domains, and the light chain comprises a LCVR and a light chain constant region, wherein the second heavy chain and paired light chain comprise the second antigen-binding domain. In some cases, the first heavy chain or the second heavy chain, but not both, comprises a CH3 domain comprising a H435R (EU numbering) modification and a Y436F (EU numbering) modification. In some cases, the heavy chain constant region of the first heavy chain and the heavy chain constant region of the second heavy chain are of isotype IgG1. In some cases, the heavy chain constant region of the first heavy chain and the heavy chain constant region of the second heavy chain are of isotype IgG4. In some cases, the first heavy chain and the second heavy chain comprise a chimeric hinge that reduces Fcγ receptor binding relative to a wild-type hinge of the same isotype. In some embodiments, the first heavy chain comprises the amino acid sequence of SEQ ID NO: 26, the second heavy chain comprises the amino acid sequence of SEQ ID NO: 28, and the paired light chain comprises the amino acid sequence of SEQ ID NO: 30.

In some embodiments, the first antigen-binding domain of the bispecific antigen-binding molecule comprises a HCVR comprising HCDR1, HCDR2 and HCDR3 comprising the amino acid sequences of SEQ ID NOs: 12, 44 and 46, respectively, and a LCVR comprising LCDR1, LCDR2 and LCDR3 comprising the amino acid sequences of SEQ ID NOs: 20, 22 and 24, respectively, and the second antigen-binding domain of the bispecific antigen-binding molecule comprises a HCVR comprising HCDR1, HCDR2 and HCDR3 comprising the amino acid sequences of SEQ ID NOs: 10, 12 and 14, respectively, and a LCVR comprising LCDR1, LCDR2 and LCDR3 comprising the amino acid sequences of SEQ ID NOs: 20, 22 and 24, respectively. In some cases, the first antigen-binding domain comprises a HCVR comprising the amino acid sequence of SEQ ID NO: 42 and a LCVR comprising the amino acid sequence of SEQ ID NO: 18, and the second antigen-binding domain comprises a HCVR comprising the amino acid sequence of SEQ ID NO: 10 and a LCVR comprising the amino acid sequence of SEQ ID NO: 18. In some embodiments, the bispecific antigen-binding molecule is a bispecific antibody comprising: a first heavy chain and a paired light chain interconnected by disulfide bonds, wherein the first heavy chain comprises a HCVR and a heavy chain constant region comprising CH1, CH2, and CH3 domains, and the paired light chain comprises a LCVR and a light chain constant region, wherein the first heavy chain and paired light chain comprise the first antigen-binding domain; and a second heavy chain and a paired light chain interconnected by disulfide bonds, wherein the second heavy chain comprises a HCVR and a heavy chain constant region comprising CH1, CH2, and CH3 domains, and the light chain comprises a LCVR and a light chain constant region, wherein the second heavy chain and paired light chain comprise the second antigen-binding domain. In some cases, the first heavy chain or the second heavy chain, but not both, comprises a CH3 domain comprising a H435R (EU numbering) modification and a Y436F (EU numbering) modification. In some cases, the heavy chain constant region of the first heavy chain and the heavy chain constant region of the second heavy chain are of isotype IgG1. In some cases, the heavy chain constant region of the first heavy chain and the heavy chain constant region of the second heavy chain are of isotype IgG4. In some cases, the first heavy chain and the second heavy chain comprise a chimeric hinge that reduces Fcγ receptor binding relative to a wild-type hinge of the same isotype. In some embodiments, the first heavy chain comprises the amino acid sequence of SEQ ID NO: 48, the second heavy chain comprises the amino acid sequence of SEQ ID NO: 28, and the paired light chain comprises the amino acid sequence of SEQ ID NO: 30.

In some embodiments, the first antigen-binding domain of the bispecific antigen-binding molecule comprises a HCVR comprising HCDR1, HCDR2 and HCDR3 comprising the amino acid sequences of SEQ ID NOs: 34, 36 and 38, respectively, and a LCVR comprising LCDR1, LCDR2 and LCDR3 comprising the amino acid sequences of SEQ ID NOs: 20, 22 and 24, respectively, and the second antigen-binding domain of the bispecific antigen-binding molecule comprises a HCVR comprising HCDR1, HCDR2 and HCDR3 comprising the amino acid sequences of SEQ ID NOs: 10, 12 and 14, respectively, and a LCVR comprising LCDR1, LCDR2 and LCDR3 comprising the amino acid sequences of SEQ ID NOs: 20, 22 and 24, respectively. In some cases, the first antigen-binding domain comprises a HCVR comprising the amino acid sequence of SEQ ID NO: 32 and a LCVR comprising the amino acid sequence of SEQ ID NO: 18, and the second antigen-binding domain comprises a HCVR comprising the amino acid sequence of SEQ ID NO: 10 and a LCVR comprising the amino acid sequence of SEQ ID NO: 18. In some embodiments, the bispecific antigen-binding molecule is a bispecific antibody comprising: a first heavy chain and a paired light chain interconnected by disulfide bonds, wherein the first heavy chain comprises a HCVR and a heavy chain constant region comprising CH1, CH2, and CH3 domains, and the paired light chain comprises a LCVR and a light chain constant region, wherein the first heavy chain and paired light chain comprise the first antigen-binding domain; and a second heavy chain and a paired light chain interconnected by disulfide bonds, wherein the second heavy chain comprises a HCVR and a heavy chain constant region comprising CH1, CH2, and CH3 domains, and the light chain comprises a LCVR and a light chain constant region, wherein the second heavy chain and paired light chain comprise the second antigen-binding domain. In some cases, the first heavy chain or the second heavy chain, but not both, comprises a CH3 domain comprising a H435R (EU numbering) modification and a Y436F (EU numbering) modification. In some cases, the heavy chain constant region of the first heavy chain and the heavy chain constant region of the second heavy chain are of isotype IgG1. In some cases, the heavy chain constant region of the first heavy chain and the heavy chain constant region of the second heavy chain are of isotype IgG4. In some cases, the first heavy chain and the second heavy chain comprise a chimeric hinge that reduces Fcγ receptor binding relative to a wild-type hinge of the same isotype. In some embodiments, the first heavy chain comprises the amino acid sequence of SEQ ID NO: 40, the second heavy chain comprises the amino acid sequence of SEQ ID NO: 28, and the paired light chain comprises the amino acid sequence of SEQ ID NO: 30.

In one aspect, the present disclosure provides a pharmaceutical composition comprising a bispecific antigen-binding molecule as discussed above or herein, and a pharmaceutically acceptable carrier or diluent.

In one aspect, the present disclosure provides a nucleic acid molecule comprising a nucleotide sequence encoding a bispecific antigen-binding molecule as discussed above or herein, or a group of nucleic acid molecules comprising nucleotide sequences, respectively, encoding the HCVR of the first antigen-binding domain, the HCVR of the second antigen-binding domain, and the LCVR of the first and second antigen-binding domains of a bispecific antigen-binding molecule as discussed above or herein.

In one aspect, the present disclosure provides an expression vector comprising the nucleic acid molecule, or a group of expression vectors comprising, respectively, the group of nucleic acid molecules, as discussed above or herein.

In one aspect, the present disclosure provides a host cell comprising a bispecific antigen-binding molecule as discussed above or herein, a nucleic acid molecule or group of nucleic acid molecules as discussed above or herein, or an expression vector or group of expression vectors as discussed above or herein.

In one aspect, the present disclosure provides a host cell comprising: (a) an expression vector comprising a nucleic acid molecule encoding a first immunoglobulin heavy chain comprising the amino acid sequence of SEQ ID NO: 26; (b) an expression vector comprising a nucleic acid molecule encoding a second immunoglobulin heavy chain comprising the amino acid sequence of SEQ ID NO: 28; and (c) an expression vector comprising a nucleic acid molecule encoding an immunoglobulin light chain comprising the amino acid sequence of SEQ ID NO: 30.

In one aspect, the present disclosure provides a host cell comprising: (a) an expression vector comprising a nucleic acid molecule encoding a first immunoglobulin heavy chain comprising the amino acid sequence of SEQ ID NO: 48; (b) an expression vector comprising a nucleic acid molecule encoding a second immunoglobulin heavy chain comprising the amino acid sequence of SEQ ID NO: 28; and (c) an expression vector comprising a nucleic acid molecule encoding an immunoglobulin light chain comprising the amino acid sequence of SEQ ID NO: 30.

In one aspect, the present disclosure provides a host cell comprising: (a) an expression vector comprising a nucleic acid molecule encoding a first immunoglobulin heavy chain comprising the amino acid sequence of SEQ ID NO: 40; (b) an expression vector comprising a nucleic acid molecule encoding a second immunoglobulin heavy chain comprising the amino acid sequence of SEQ ID NO: 28; and (c) an expression vector comprising a nucleic acid molecule encoding an immunoglobulin light chain comprising the amino acid sequence of SEQ ID NO: 30.

In one aspect, the present disclosure provides a method of producing a bispecific antigen-binding molecule as discussed above or herein, comprising culturing a host cell as discussed above or herein under conditions permitting production of the bispecific antigen-binding molecule, and recovering the bispecific antigen-binding molecule so produced. In some embodiments, the method further comprises formulating the bispecific antigen-binding molecule as a pharmaceutical composition with a suitable carrier.

In one aspect, the present disclosure provides a method of treating a GPRC5D-expressing cancer in a subject in need thereof, the method comprising administering a bispecific antigen-binding molecule as discussed above or herein, or a pharmaceutical composition as discussed above or herein, to the subject. In some embodiments, the GPRC5D-expressing cancer is multiple myeloma. In some embodiments, subject has relapsed following prior therapy to treat the GPRC5D-expressing cancer, or the subject is refractory to at least one other therapy to treat the GPRC5D-expressing cancer. In some embodiments, the GPRC5D-expressing cancer is multiple myeloma, non-small-cell lung cancer, head & neck cancer, breast cancer, colorectal cancer, pancreatic cancer, ovarian cancer, non-Hodgkin's lymphoma, renal cell carcinoma, lung cancer, liver cancer, or stomach cancer.

In one aspect, the present disclosure provides a method of reducing risk of progression to symptomatic multiple myeloma in a subject in need thereof, comprising administering to the subject a bispecific antigen-binding molecule as discussed above or herein or a pharmaceutical composition as discussed above or herein to a subject that has been diagnosed with monoclonal gammopathy of undetermined significance (MGUS) or smoldering multiple myeloma (SMM) such that the risk of progression is reduced relative to a control population not administered the bispecific antigen-binding molecule. In some embodiments, the subject is a subject that has been diagnosed with MGUS. In some such cases, the subject has a serum monoclonal protein level of <3 g/dL, clonal bone marrow plasma cells of <10%, and an absence of end-organ damage or a myeloma-defining event that can be attributed to the underlying plasma cell proliferative disorder. In some embodiments, the subject is a subject that has been diagnosed with SMM. In some such cases, the subject has a serum monoclonal protein level of ≥3 g/dL, clonal bone marrow plasma cells of ≥10%, and an absence of end-organ damage or a myeloma-defining event that can be attributed to the underlying plasma cell proliferative disorder. In various embodiments, the subject may exhibit one or more indicia of an enhanced risk of progression to symptomatic multiple myeloma. In some embodiments, the methods further comprise identifying a subject that exhibits one or more indicia of an enhanced risk of progression to symptomatic multiple myeloma. In some cases, the one or more indicia of an enhanced risk of progression to symptomatic multiple myeloma includes one or more of: (a) an M protein level of ≥5 g/L, ≥15 g/L, or ≥25 g/L; (b) presence of an IgM or IgA monoclonal protein; (c) presence of >5% clonal bone marrow plasma cells; and (d) an abnormal FLC ratio.

In some embodiments of the methods discussed above or herein, the method further comprises administering a second therapeutic agent or therapy. In some cases, the second therapeutic agent or therapy comprises a chemotherapeutic drug, DNA alkylators, immunomodulators, proteasome inhibitors, histone deacetylase inhibitors, radiotherapy, a stem cell transplant, a different bispecific antigen-binding molecule that interacts with a different tumor cell surface antigen and a T cell or immune cell antigen, an antibody drug conjugate, a bispecific antibody that specifically binds human B-cell maturation antigen (BCMA) and human CD3, a PD-1 inhibitor, a PD-L1 inhibitor, a CTLA-4 inhibitor, a LAG-3 inhibitor, or combinations thereof. In some embodiments, the second therapeutic agent or therapy comprises a bispecific antibody that specifically binds human BCMA and human CD3. In some embodiments, the bispecific antibody that specifically binds human BCMA and human CD3 is linvoseltamab.

In one aspect, the present disclosure provides an isolated antibody or antigen binding fragment thereof that specifically binds to human G protein-coupled receptor, class C group 5 member D (GPRC5D), wherein the antibody or antigen-binding fragment thereof comprises a heavy chain variable region (HCVR) comprising three heavy chain complementarity determining regions HCDR1, HCDR2 and HCDR3, and a light chain variable region (LCVR) comprising three light chain complementarity determining regions, LCDR1, LCDR2 and LCDR3, wherein the HCDR1, HCDR2 and HCDR3 are the complementarity determining regions contained within a HCVR comprising the amino acid sequence of SEQ ID NO: 2, 32 or 42, and the LCDR1, LCDR2 and LCDR3 are the complementarity determining regions contained within a LCVR comprising the amino acid sequence of SEQ ID NO: 18.

In some embodiments, the antibody or antigen-binding fragment thereof binds to human GPRC5D within residues comprising an extracellular loop (ECL) 2 and ECL3 of GPRC5D.

In some embodiments, the antibody or antigen-binding fragment comprises HCDR1, HCDR2, HCDR3, LCDR1, LCDR2, and LCDR3 domains comprising the amino acid sequences of SEQ ID NOs: 4, 6, 8, 20, 22, and 24, respectively. In some embodiments, the antibody or antigen-binding fragment comprises a HCVR comprising the amino acid sequence of SEQ ID NO: 2 and a LCVR comprising the amino acid sequence of SEQ ID NO: 18. In some embodiments, the antibody or antigen-binding fragment comprises a heavy chain comprising the amino acid sequence of SEQ ID NO: 26 and a light chain comprising the amino acid sequence of SEQ ID NO: 30.

In some embodiments, the antibody or antigen-binding fragment comprises HCDR1, HCDR2, HCDR3, LCDR1, LCDR2, and LCDR3 domains comprising the amino acid sequences of SEQ ID NOs: 34, 36, 38, 20, 22, and 24, respectively. In some embodiments, the antibody or antigen-binding fragment comprises a HCVR comprising the amino acid sequence of SEQ ID NO: 32 and a LCVR comprising the amino acid sequence of SEQ ID NO: 18. In some embodiments, the antibody or antigen-binding fragment comprises a heavy chain comprising the amino acid sequence of SEQ ID NO: 40 and a light chain comprising the amino acid sequence of SEQ ID NO: 30.

In some embodiments, the antibody or antigen-binding fragment comprises HCDR1, HCDR2, HCDR3, LCDR1, LCDR2, and LCDR3 domains comprising the amino acid sequences of SEQ ID NOs: 12, 44, 46, 20, 22, and 24, respectively. In some embodiments, the antibody or antigen-binding fragment comprises a HCVR comprising the amino acid sequence of SEQ ID NO: 42 and a LCVR comprising the amino acid sequence of SEQ ID NO: 18. In some embodiments, the antibody or antigen-binding fragment comprises a heavy chain comprising the amino acid sequence of SEQ ID NO: 48 and a light chain comprising the amino acid sequence of SEQ ID NO: 30.

In one aspect, the present disclosure provides a pharmaceutical composition comprising the antibody or antigen-binding fragment discussed above or herein, and a pharmaceutically acceptable carrier or diluent.

In one aspect, the present disclosure provides a nucleic acid molecule comprising a nucleotide sequence encoding an antibody or antigen-binding fragment as discussed above or herein, or a pair of nucleic acid molecules comprising nucleotide sequences, respectively, encoding the HCVR and the LCVR of an antibody or antigen-binding fragment as discussed above or herein.

In one aspect, the present disclosure provides an expression vector comprising the nucleic acid molecule as discussed above or herein, or a pair of expression vectors comprising, respectively, the pair of nucleic acid molecules discussed above or herein.

In one aspect, the present disclosure provides a host cell comprising an antibody or antigen-binding fragment as discussed above or herein, a nucleic acid molecule or pair of nucleic acid molecules as discussed above or herein, or the expression vector or pair of expression vectors as discussed above or herein.

In one aspect, the present disclosure provides a method of treating a GPRC5D-expressing cancer in a subject in need thereof, the method comprising administering an antibody or antigen-binding fragment as discussed above or herein, or a pharmaceutical composition as discussed above or herein, to the subject. In some embodiments, the GPRC5D-expressing cancer is multiple myeloma.

The present disclosure further includes use of the antibodies or bispecific antigen-binding molecules discussed above or herein in the manufacture of a medicament for treating a disease or disorder associated with expression of GPRC5D, as well as use of the antibodies or bispecific antigen-binding molecules discussed above or herein for treating a disease or disorder associated with expression of GPRC5D, as well as an antibody or bispecific antigen-binding molecule as discussed above or herein for treating a disease or disorder associated with expression of GPRC5D. Thus, it will be understood that any method of treatment discussed above or herein also encompasses Swiss-use, use and product-for-use embodiments.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges and all values falling within such ranges are encompassed within the scope of the present disclosure.

Other embodiments will become apparent from a review of the ensuing detailed description.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present disclosure, the preferred methods and materials are now described. All patents, applications and non-patent publications mentioned in this specification are incorporated herein by reference in their entireties.

### Terms and Definitions

The expression “GPRC5D,” as used herein, refers to G protein-coupled receptor, class C group 5 member D. Human GPRC5D has two published isoforms: X1, corresponding to NCBI accession number XP_054228436; and X2, corresponding to NCBI accession number XP_054228435.

As used herein, “an antibody that specifically binds GPRC5D” or an “anti-GPRC5D antibody” includes antibodies and antigen-binding fragments thereof that specifically recognize GPRC5D.

The expression “CD28,” as used herein, refers to an antigen which is expressed on T cells as a costimulatory receptor. Human CD28 comprises the amino acid sequence as set forth in NCBI accession No. NP_006130.1. All references to proteins, polypeptides and protein fragments herein are intended to refer to the human version of the respective protein, polypeptide or protein fragment unless explicitly specified as being from a non-human species. Thus, the expression “CD28” means human CD28 unless specified as being from a non-human species, e.g., “mouse CD28” “monkey CD28.”

As used herein, “an antibody that specifically binds CD28” or an “anti-CD28 antibody” includes antibodies and antigen-binding fragments thereof that specifically recognize a monomeric CD28, as well as antibodies and antigen-binding fragments thereof that specifically recognize a dimeric CD28. The antibodies and antigen-binding fragments of the present disclosure may bind soluble CD28 and/or cell surface expressed CD28. Soluble CD28 includes natural CD28 proteins as well as recombinant CD28 protein variants such as, e.g., monomeric and dimeric CD28 constructs, that lack a transmembrane domain or are otherwise unassociated with a cell membrane.

As used herein, the expression “cell surface-expressed CD28” means one or more CD28 protein(s) that is/are expressed on the surface of a cell in vitro or in vivo, such that at least a portion of a CD28 protein is exposed to the extracellular side of the cell membrane and is accessible to an antigen-binding portion of an antibody. “Cell surface-expressed CD28” includes CD28 proteins contained within the context of a functional T cell costimulatory receptor in the membrane of a cell. The expression “cell surface-expressed CD28” includes CD28 protein expressed as part of a homodimer on the surface of a cell. A “cell surface-expressed CD28” can comprise or consist of a CD28 protein expressed on the surface of a cell which normally expresses CD28 protein. Alternatively, “cell surface-expressed CD28” can comprise or consist of CD28 protein expressed on the surface of a cell that normally does not express human CD28 on its surface but has been artificially engineered to express CD28 on its surface.

The term “antigen-binding molecule” includes antibodies and antigen-binding fragments of antibodies, including, e.g., bispecific antibodies and antigen-binding fragments thereof.

The term “antibody”, as used herein, means any antigen-binding molecule or molecular complex comprising at least one complementarity determining region (CDR) that specifically binds to or interacts with a particular antigen (e.g., GPRC5D or CD28). The term “antibody” includes immunoglobulin molecules comprising four polypeptide chains, two heavy (H) chains and two light (L) chains inter-connected by disulfide bonds, as well as multimers thereof (e.g., IgM). The term “antibody” also includes immunoglobulin molecules consisting of four polypeptide chains, two heavy (H) chains and two light (L) chains inter-connected by disulfide bonds. Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-GPRC5D antibody or anti-CD28 antibody (or antigen-binding portion thereof) may be identical to the human germline sequences, or may be naturally or artificially modified. An amino acid consensus sequence may be defined based on a side-by-side analysis of two or more CDRs.

The term “antibody”, as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present disclosure using routine techniques available in the art.

The antibodies of the present disclosure may function through complement-dependent cytotoxicity (CDC) or antibody-dependent cell-mediated cytotoxicity (ADCC). “Complement-dependent cytotoxicity” (CDC) refers to lysis of antigen-expressing cells by an antibody of the disclosure in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al. (1998) Proc. Natl. Acad. Sci. (USA) 95:652-656). The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

In certain embodiments of the disclosure, the anti-GPRC5D monospecific antibodies or anti-GPRC5D x anti-CD28 bispecific antibodies of the disclosure are human antibodies. The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The antibodies of the disclosure may, in some embodiments, be recombinant human antibodies. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al., Nucl. Acids Res., 20:6287-6295, 1992) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al., Molecular Immunology 30:105, 1993) to levels typically observed using a human IgG1 hinge. The instant disclosure encompasses antibodies having one or more mutations in the hinge, C_{H}2 or C_{H}3 region which may be desirable, for example, in production, to improve the yield of the desired antibody form.

The antibodies of the disclosure may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present disclosure. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The present disclosure also includes one-arm antibodies that bind GPRC5D. As used herein, a “one-arm antibody” means an antigen-binding molecule comprising a single antibody heavy chain and a single antibody light chain. The one-arm antibodies of the present disclosure may comprise any of the HCVR/LCVR or CDR amino acid sequences as set forth in Table 1A.

The anti-GPRC5D or anti-GPRC5D x anti-CD28 antibodies disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The present disclosure also includes anti-GPRC5D or anti-GPRC5D x anti-CD28 antibodies comprising variants of any of the HCVR, LCVR, and/or CDR amino acid sequences disclosed herein having one or more conservative substitutions. For example, the present disclosure includes anti-GPRC5D or anti-GPRC5D x anti-CD28 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in Tables 1 and 3 herein. In some embodiments, the present disclosure also includes anti-GPRC5D or anti-GPRC5D x anti-CD28 antibodies that have 90%, 95%, 96%, 97%, 98%, or 99% sequence identity with any of the HCVR, LCVR, HC, or LC sequences set forth in Tables 1 and 3 herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson, Methods Mol. Biol., 24:307-331, 1994, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al., Science 256:1443-1445, 1992, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al., J. Mol. Biol. 215:403-410, 1990 and Altschul et al., Nucleic Acids Res. 25:3389-402, 1997, each herein incorporated by reference.

As used herein, the term “binding” in the context of the binding of an antibody, immunoglobulin, antibody-binding fragment, or Fc-containing protein to either, e.g., a predetermined antigen, such as a cell surface protein or fragment thereof, typically refers to an interaction or association between a minimum of two entities or molecular structures, such as an antibody-antigen interaction.

For instance, binding affinity typically corresponds to a K_{D} value of about 10⁻⁷ M or less, such as about 10⁻⁸ M or less, such as about 10⁻⁹ M or less when determined by, for instance, surface plasmon resonance (SPR) technology in a BIAcore 3000 instrument using the antigen as the ligand and the antibody, Ig, antibody-binding fragment, or Fc-containing protein as the analyte (or antiligand). Cell-based binding strategies, such as fluorescent-activated cell sorting (FACS) binding assays, are also routinely used, and FACS data correlates well with other methods such as radioligand competition binding and SPR (Benedict, C A, J Immunol Methods, 201(2):223-31, 1997; Geuijen, C A, et al. J Immunol Methods., 302 (1-2): 68-77, 2005).

Accordingly, the antibody or antigen-binding protein of the disclosure binds to the predetermined antigen or cell surface molecule (receptor) having an affinity corresponding to a K_{D} value that is at least ten-fold lower than its affinity for binding to a non-specific antigen (e.g., BSA, casein). According to the present disclosure, the affinity of an antibody corresponding to a K_{D} value that is equal to or less than ten-fold lower than a non-specific antigen may be considered non-detectable binding, however such an antibody may be paired with a second antigen binding arm for the production of a bispecific antibody of the disclosure.

The term “K_{D}” (M) refers to the dissociation equilibrium constant of a particular antibody-antigen interaction, or the dissociation equilibrium constant of an antibody or antibody-binding fragment binding to an antigen. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g. antibody) to its interactive partner molecule (e.g. antigen X) compared to the binding affinity of the molecule (e.g. antibody) to another interactive partner molecule (e.g. antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be.

The term “k_{d}” (sec−1 or 1/s) refers to the dissociation rate constant of a particular antibody-antigen interaction, or the dissociation rate constant of an antibody or antibody-binding fragment. Said value is also referred to as the k_{off} value.

The term “kₐ” (M−1×sec−1 or 1/M) refers to the association rate constant of a particular antibody-antigen interaction, or the association rate constant of an antibody or antibody-binding fragment.

The term “K_{A}” (M−1 or 1/M) refers to the association equilibrium constant of a particular antibody-antigen interaction, or the association equilibrium constant of an antibody or antibody-binding fragment. The association equilibrium constant is obtained by dividing the ka by the ka.

The term “EC50” or “EC₅₀” refers to the half maximal effective concentration, which includes the concentration of an antibody which induces a response halfway between the baseline and maximum after a specified exposure time. The EC₅₀ essentially represents the concentration of an antibody where 50% of its maximal effect is observed. In certain embodiments, the EC₅₀ value equals the concentration of an antibody of the disclosure that gives half-maximal binding to cells expressing CD28 or GPRC5D, as determined by e.g. a FACS binding assay. Thus, reduced or weaker binding is observed with an increased EC₅₀, or half maximal effective concentration value.

In one embodiment, decreased binding can be defined as an increased EC₅₀ antibody concentration which enables binding to the half-maximal amount of target cells.

In another embodiment, the EC₅₀ value represents the concentration of an antibody of the disclosure that elicits half-maximal depletion of target cells by T cell cytotoxic activity. Thus, increased cytotoxic activity (e.g. T cell-mediated tumor cell killing) is observed with a decreased EC₅₀, or half maximal effective concentration value.

A “plasma cell” is a differentiated B-lymphocyte capable of secreting antibodies.

References to “end-organ damage or a myeloma-defining event that can be attributed to an underlying plasma cell proliferative disorder” for defining symptomatic multiple myeloma include one or more of the following: (i) hypercalcemia, wherein serum calcium is >11 mg/dL; (ii) renal insufficiency, wherein serum creatinine is >177 μmol/L; (iii) anemia with a hemoglobin value ≥2 g/dL below the lower limit of normal, or a hemoglobin value of <10 g/dL; (iv) more than one myeloma-related lesion on advanced imaging, including magnetic resonance imaging (MRI), positron emission tomography/computed tomography (PET/CT), or whole-body low dose computed tomography (WBLDCT); (v) clonal bone marrow plasma cells ≥60%; or (vi) involved/uninvolved serum free light chain (FLC) ratio of 100 or more.

### Bispecific Antigen-Binding Molecules

The bispecific antigen-binding molecules of the present disclosure may be monospecific, bi-specific, or multispecific. Multispecific antigen-binding molecules may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The anti-GPRC5D monospecific antibodies or anti-GPRC5D x anti-CD28 bispecific antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second or additional binding specificity.

Use of the expression “anti-CD28 antibody” or “anti-GPRC5D antibody” herein is intended to include both monospecific anti-CD28 or anti-GPRC5D antibodies as well as bispecific antibodies comprising a CD28-binding arm and a GPRC5D-binding arm. Thus, the present disclosure includes bispecific antibodies wherein one arm of an immunoglobulin binds human CD28, and the other arm of the immunoglobulin is specific for human GPRC5D. The CD28-binding arm can comprise the HC/LC, HCVR/LCVR or CDR amino acid sequences as set forth in Table 3 herein.

In certain embodiments, the CD28-binding arm binds to human CD28 and induces human T cell activation. In certain embodiments, the CD28-binding arm binds weakly to human CD28 and induces human T cell activation. In other embodiments, the CD28-binding arm binds weakly to human CD28 and induces tumor-associated antigen-expressing cell killing in the context of a bispecific or multispecific antibody. The GPRC5D-binding arm can comprise any of the HCVR/LCVR or CDR amino acid sequences as set forth in Table 1A herein.

According to certain exemplary embodiments, the present disclosure includes bispecific antigen-binding molecules that specifically bind CD28 and GPRC5D. Such molecules may be referred to herein as, e.g., “anti-GPRC5D x anti-CD28” or “anti-CD28/anti-GPRC5D,” or “anti-CD28xGPRC5D” bispecific molecules, or other similar terminology (e.g., anti-GPRC5D/anti-CD28).

The term “GPRC5D,” as used herein, refers to the human GPRC5D protein unless specified as being from a non-human species (e.g., “mouse GPRC5D,” “monkey GPRC5D,” etc.).

As used herein, the expression “antigen-binding molecule” means a protein, polypeptide or molecular complex comprising or consisting of at least one complementarity determining region (CDR) that alone, or in combination with one or more additional CDRs and/or framework regions (FRs), specifically binds to a particular antigen. In certain embodiments, an antigen-binding molecule is an antibody or a fragment of an antibody, as those terms are defined elsewhere herein.

As used herein, the expression “bispecific antigen-binding molecule” means a protein, polypeptide or molecular complex comprising at least a first antigen-binding domain and a second antigen-binding domain. Each antigen-binding domain within the bispecific antigen-binding molecule comprises at least one CDR that alone, or in combination with one or more additional CDRs and/or FRs, specifically binds to a particular antigen. In the context of the present disclosure, the first antigen-binding domain specifically binds a first antigen (e.g., GPRC5D), and the second antigen-binding domain specifically binds a second, distinct antigen (e.g., CD28).

In certain exemplary embodiments of the present disclosure, the bispecific antigen-binding molecule is a bispecific antibody. Each antigen-binding domain of a bispecific antibody comprises a heavy chain variable domain (HCVR) and a light chain variable domain (LCVR). In the context of a bispecific antigen-binding molecule comprising a first and a second antigen-binding domain (e.g., a bispecific antibody), the CDRs of the first antigen-binding domain may be designated with the prefix “D1” and the CDRs of the second antigen-binding domain may be designated with the prefix “D2”. Thus, the CDRs of the first antigen-binding domain may be referred to herein as D1-HCDR1, D1-HCDR2, and D1-HCDR3; and the CDRs of the second antigen-binding domain may be referred to herein as D2-HCDR1, D2-HCDR2, and D2-HCDR3. In some embodiments, the bispecific antibody comprises a first heavy chain paired with a light chain, and a second heavy chain paired with a light chain (the light chain may be common to both heavy chains, i.e., two separate, but identical light chains, each separately paired with one of the heavy chains). The two heavy chains each comprise a HCVR comprising HCDR1, HCDR2 and HCDR3 in framework regions, a CH1 domain, a hinge, a CH2 domain, and a CH3 domain. The two light chain each comprise a LCVR comprising LCDR1, LCDR2 and LCDR3 in framework regions, and a light chain constant (CL) domain. In the context of the present disclosure, the bispecific antibody is human as defined elsewhere herein.

In certain exemplary embodiments, the bispecific antigen-binding molecule comprises a first antigen-binding domain that comprises: (a) three heavy chain complementarity determining regions (HCDR1, HCDR2 and HCDR3) contained within a heavy chain variable region (HCVR) comprising the amino acid sequence of SEQ ID NO: 2; and (b) three light chain complementarity determining regions (LCDR1, LCDR2 and LCDR3) contained within a light chain variable region (LCVR) comprising the amino acid sequence of SEQ ID NO: 18. In some cases, the isolated bispecific antigen binding molecule comprises a HCDR1 comprising the amino acid sequence of SEQ ID NO: 4, a HCDR2 comprising the amino acid sequence of SEQ ID NO: 6, and a HCDR3 comprising the amino acid sequence of SEQ ID NO: 8. In some cases, the isolated bispecific antigen-binding molecule comprises a LCDR1 comprising the amino acid sequence of SEQ ID NO: 20, a LCDR2 comprising the amino acid sequence of SEQ ID NO: 22, and a LCDR3 comprising the amino acid sequence of SEQ ID NO: 24. In some cases, the first antigen-binding domain comprises a HCVR comprising the amino acid sequence of SEQ ID NO: 2, and a LCVR comprising the amino acid sequence of SEQ ID NO: 18.

In certain exemplary embodiments, the bispecific antigen-binding molecule comprises a first antigen-binding domain that comprises: (a) three heavy chain complementarity determining regions (HCDR1, HCDR2 and HCDR3) contained within a heavy chain variable region (HCVR) comprising the amino acid sequence of SEQ ID NO: 32; and (b) three light chain complementarity determining regions (LCDR1, LCDR2 and LCDR3) contained within a light chain variable region (LCVR) comprising the amino acid sequence of SEQ ID NO: 18. In some cases, the isolated bispecific antigen binding molecule comprises a HCDR1 comprising the amino acid sequence of SEQ ID NO: 34, a HCDR2 comprising the amino acid sequence of SEQ ID NO: 36, and a HCDR3 comprising the amino acid sequence of SEQ ID NO: 38. In some cases, the isolated bispecific antigen-binding molecule comprises a LCDR1 comprising the amino acid sequence of SEQ ID NO: 20, a LCDR2 comprising the amino acid sequence of SEQ ID NO: 22, and a LCDR3 comprising the amino acid sequence of SEQ ID NO: 24. In some cases, the first antigen-binding domain comprises a HCVR comprising the amino acid sequence of SEQ ID NO: 32, and a LCVR comprising the amino acid sequence of SEQ ID NO: 18.

In certain exemplary embodiments, the bispecific antigen-binding molecule comprises a first antigen-binding domain that comprises: (a) three heavy chain complementarity determining regions (HCDR1, HCDR2 and HCDR3) contained within a heavy chain variable region (HCVR) comprising the amino acid sequence of SEQ ID NO: 42; and (b) three light chain complementarity determining regions (LCDR1, LCDR2 and LCDR3) contained within a light chain variable region (LCVR) comprising the amino acid sequence of SEQ ID NO: 18. In some cases, the isolated bispecific antigen binding molecule comprises a HCDR1 comprising the amino acid sequence of SEQ ID NO: 12, a HCDR2 comprising the amino acid sequence of SEQ ID NO: 44, and a HCDR3 comprising the amino acid sequence of SEQ ID NO: 46. In some cases, the isolated bispecific antigen-binding molecule comprises a LCDR1 comprising the amino acid sequence of SEQ ID NO: 20, a LCDR2 comprising the amino acid sequence of SEQ ID NO: 22, and a LCDR3 comprising the amino acid sequence of SEQ ID NO: 24. In some cases, the first antigen-binding domain comprises a HCVR comprising the amino acid sequence of SEQ ID NO: 42, and a LCVR comprising the amino acid sequence of SEQ ID NO: 18.

In certain exemplary embodiments, the bispecific antigen-binding molecule comprises a second antigen-binding domain that comprises: (a) three heavy chain complementarity determining regions (HCDR1, HCDR2 and HCDR3) contained within a heavy chain variable region (HCVR) comprising the amino acid sequence of SEQ ID NO: 10; and (b) three light chain complementarity determining regions (LCDR1, LCDR2 and LCDR3) contained within a light chain variable region (LCVR) comprising the amino acid sequence of SEQ ID NO: 18. In some cases, the second antigen-binding domain comprises: (a) a HCDR1 comprising the amino acid sequence of SEQ ID NO: 12; (b) a HCDR2 comprising the amino acid sequence of SEQ ID NO: 14; and (c) a HCDR3 comprising the amino acid sequence of SEQ ID NO: 16. In some cases, the second antigen-binding domain comprises a LCDR1 comprising the amino acid sequence of SEQ ID NO: 20, a LCDR2 comprising the amino acid sequence of SEQ ID NO: 22, and a LCDR3 comprising the amino acid sequence of SEQ ID NO: 24. In some cases, the second antigen-binding domain comprises a HCVR comprising the amino acid sequence of SEQ ID NO: 10, and a LCVR comprising the amino acid sequence of SEQ ID NO: 18.

In certain exemplary embodiments, the bispecific antigen-binding molecule comprises: (a) a first antigen-binding domain that comprises HCDR1, HCDR2, HCDR3 domains, respectively, comprising the amino acid sequences of SEQ ID NOs: 4, 6, 8, and LCDR1, LCDR2, LCDR3 domains, respectively, comprising the amino acid sequences of SEQ ID NOs: 20, 22, 24; and (b) a second antigen binding domain that comprises HCDR1, HCDR2, HCDR3 domains, respectively, comprising the amino acid sequences of SEQ ID NOs: 12, 14, 16, and LCDR1, LCDR2, LCDR3 domains, respectively, comprising the amino acid sequences of SEQ ID NOs: 20, 22, 24. In some cases, the bispecific antigen-binding molecule comprises: (a) a first antigen binding domain that comprises a HCVR comprising the amino acid sequence of SEQ ID NO: 2, and a LCVR comprising the amino acid sequence of SEQ ID NO: 18; and (b) a second antigen binding domain that comprises a HCVR comprising the amino acid sequence of SEQ ID NO: 10, and a LCVR comprising the amino acid sequence of SEQ ID NO: 18. In some cases, the bispecific antigen-binding molecule comprises a first heavy chain comprising the amino acid sequence of SEQ ID NO: 26, a second heavy chain comprising the amino acid sequence of SEQ ID NO: 28, and a common light chain comprising the amino acid sequence of SEQ ID NO: 30.

In certain exemplary embodiments, the bispecific antigen-binding molecule comprises: (a) a first antigen-binding domain that comprises HCDR1, HCDR2, HCDR3 domains, respectively, comprising the amino acid sequences of SEQ ID NOs: 34, 36, 38, and LCDR1, LCDR2, LCDR3 domains, respectively, comprising the amino acid sequences of SEQ ID NOs: 20, 22, 24; and (b) a second antigen binding domain that comprises HCDR1, HCDR2, HCDR3 domains, respectively, comprising the amino acid sequences of SEQ ID NOs: 12, 14, 16, and LCDR1, LCDR2, LCDR3 domains, respectively, comprising the amino acid sequences of SEQ ID NOs: 20, 22, 24. In some cases, the bispecific antigen-binding molecule comprises: (a) a first antigen binding domain that comprises a HCVR comprising the amino acid sequence of SEQ ID NO: 32, and a LCVR comprising the amino acid sequence of SEQ ID NO: 18; and (b) a second antigen binding domain that comprises a HCVR comprising the amino acid sequence of SEQ ID NO: 10, and a LCVR comprising the amino acid sequence of SEQ ID NO: 18. In some cases, the bispecific antigen-binding molecule comprises a first heavy chain comprising the amino acid sequence of SEQ ID NO: 40, a second heavy chain comprising the amino acid sequence of SEQ ID NO: 28, and a common light chain comprising the amino acid sequence of SEQ ID NO: 30.

In certain exemplary embodiments, the bispecific antigen-binding molecule comprises: (a) a first antigen-binding domain that comprises HCDR1, HCDR2, HCDR3 domains, respectively, comprising the amino acid sequences of SEQ ID NOs: 12, 44, 46, and LCDR1, LCDR2, LCDR3 domains, respectively, comprising the amino acid sequences of SEQ ID NOs: 20, 22, 24; and (b) a second antigen binding domain that comprises HCDR1, HCDR2, HCDR3 domains, respectively, comprising the amino acid sequences of SEQ ID NOs: 12, 14, 16, and LCDR1, LCDR2, LCDR3 domains, respectively, comprising the amino acid sequences of SEQ ID NOs: 20, 22, 24. In some cases, the bispecific antigen-binding molecule comprises: (a) a first antigen binding domain that comprises a HCVR comprising the amino acid sequence of SEQ ID NO: 42, and a LCVR comprising the amino acid sequence of SEQ ID NO: 18; and (b) a second antigen binding domain that comprises a HCVR comprising the amino acid sequence of SEQ ID NO: 10, and a LCVR comprising the amino acid sequence of SEQ ID NO: 18. In some cases, the bispecific antigen-binding molecule comprises a first heavy chain comprising the amino acid sequence of SEQ ID NO: 48, a second heavy chain comprising the amino acid sequence of SEQ ID NO: 28, and a common light chain comprising the amino acid sequence of SEQ ID NO: 30.

In certain exemplary embodiments, the isolated bispecific antigen binding molecule competes for binding to GPRC5D, or binds to the same epitope on GPRC5D as a reference antibody, wherein the reference antibody comprises a first antigen-binding domain comprising an HCVR/LCVR pair comprising the amino acid sequences of SEQ ID NOs: 2/18, 32/18, or 42/18 and a second antigen-binding domain comprising an HCVR/LCVR pair comprising the amino acid sequences of either SEQ ID NOs: 10/18. In some cases, the reference antibody is any of the anti-GPRC5D antibodies discussed herein.

In certain exemplary embodiments, the isolated bispecific antigen binding molecule competes for binding to human CD28, or binds to the same epitope on human CD28 as a reference antibody, wherein the reference antibody comprises a first antigen-binding domain comprising an HCVR/LCVR pair comprising the amino acid sequences of SEQ ID NOs: 2/18, 32/18, or 42/18 and a second antigen-binding domain comprising an HCVR/LCVR pair comprising the amino acid sequences of either SEQ ID NOs: 10/18. In some cases, the reference antibody is any of the anti-CD28 antibodies discussed herein.

The bispecific antigen-binding molecules discussed above or herein may be bispecific antibodies. In some cases, the bispecific antibody comprises a human IgG heavy chain constant region. In some cases, the human IgG heavy chain constant region is isotype IgG1. In some cases, the human IgG heavy chain constant region is isotype IgG4. In various embodiments, the bispecific antibody comprises a chimeric hinge that reduces Fcγ receptor binding relative to a wild-type hinge of the same isotype. In certain embodiments, the heavy chain constant region may be chimeric, combining sequences derived from more than one immunoglobulin isotype. For example, a chimeric heavy chain constant domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric heavy chain constant domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric heavy chain constant domain that can be included in any of the antigen-binding molecules set forth herein comprises, from N- to C-terminus: [IgG4 C_{H}1]-[IgG4 upper hinge]-[IgG2 lower hinge]-[IgG4 C_{H}2]-[IgG4 C_{H}3]. Another example of a chimeric heavy chain constant domain that can be included in any of the antigen-binding molecules set forth herein comprises, from N- to C-terminus: [IgG1 C_{H}1]-[IgG1 upper hinge]-[IgG2 lower hinge]-[IgG4 C_{H}2]-[IgG1 C_{H}3]. These and other examples of chimeric heavy chain constant domains that can be included in any of the antigen-binding molecules of the present disclosure are described in WO 2014/121087, which is herein incorporated in its entirety. Chimeric heavy chain constant domains having these general structural arrangements, and variants thereof, can have altered Fc receptor binding, which in turn affects Fc effector function.

The first antigen-binding domain and the second antigen-binding domain may be directly or indirectly connected to one another to form a bispecific antigen-binding molecule of the present disclosure. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Bispecific antigen-binding molecules of the present disclosure will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of from 1 to about 200 amino acids in length containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

Any bispecific antibody format or technology may be used to make the bispecific antigen-binding molecules of the present disclosure. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats).

In the context of bispecific antigen-binding molecules of the present disclosure, the multimerizing domains, e.g., Fc domains, may comprise one or more amino acid changes (e.g., insertions, deletions or substitutions) as compared to the wild-type, naturally occurring version of the Fc domain. For example, the disclosure includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428 L (e.g., M428 L) and 434S (e.g., N434S) modification; a 428 L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428 L modification (e.g., T250Q and M428 L); and a 307 and/or 308 modification (e.g., 308F or 308P).

The present disclosure also includes bispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. In some embodiments, one C_{H}3 domain, but not both, comprises H435R and Y436F modifications (EU numbering). Further modifications that may be found within the second C_{H}3 include: D16E, L18M, N44S, K52N, V57M, and V82I (by IMGT; D356E, L358M, N384S, K392N, V397M, and V4221 by EU) in the case of IgG1 antibodies; N44S, K52N, and V82I (IMGT; N384S, K392N, and V4221 by EU) in the case of IgG2 antibodies; and Q15R, N44S, K52N, V57M, R69K, E79Q, and V82I (by IMGT; Q355R, N384S, K392N, V397M, R409K, E419Q, and V4221 by EU) in the case of IgG4 antibodies.

### Sequence Variants

The antibodies and bispecific antigen-binding molecules of the present disclosure may comprise one or more amino acid substitutions, insertions and/or deletions in the framework and/or CDR regions of the heavy and light chain variable domains as compared to the corresponding germline sequences from which the individual antigen-binding domains were derived. Such mutations can be readily ascertained by comparing the amino acid sequences disclosed herein to germline sequences available from, for example, public antibody sequence databases. The antigen-binding molecules of the present disclosure may comprise antigen-binding domains which are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antigen-binding domains that contain one or more germline mutations can be easily tested for one or more desired property such as, improved binding specificity, increased binding affinity, improved or enhanced antagonistic or agonistic biological properties (as the case may be), reduced immunogenicity, etc. Bispecific antigen-binding molecules comprising one or more antigen-binding domains obtained in this general manner are encompassed within the present disclosure.

pH-Dependent Binding

The present disclosure includes anti-GPRC5D antibodies, and anti-GPRC5D x anti-CD28 bispecific antigen-binding molecules, with pH-dependent binding characteristics. For example, an anti-GPRC5D antibody of the present disclosure may exhibit reduced binding to GPRC5D at acidic pH as compared to neutral pH. Alternatively, anti-GPRC5D antibodies of the disclosure may exhibit enhanced binding to GPRC5D at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding . . . at acidic pH as compared to neutral pH” is expressed in terms of a ratio of the K_{D} value of the antibody binding to its antigen at acidic pH to the K_{D} value of the antibody binding to its antigen at neutral pH (or vice versa). For example, an antibody or antigen-binding fragment thereof may be regarded as exhibiting “reduced binding to GPRC5D at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0. 25.0, 30.0, 40.0, 50.0, 60.0, 70.0, 100.0 or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

### Biological Characteristics of the Antibodies and Bispecific Antigen-Binding Molecules

The present disclosure includes antibodies and antigen-binding fragments thereof that bind human GPRC5D with high affinity (e.g., nanomolar or sub-nanomolar K_{D} values).

According to certain embodiments, the present disclosure includes antibodies and antigen-binding fragments of antibodies that bind human GPRC5D (e.g., at 25° C.) with a K_{D} of less than about 5 nM as measured by surface plasmon resonance, e.g., using an assay format as defined in Example 2 herein. In certain embodiments, the antibodies or antigen-binding fragments of the present disclosure bind GPRC5D with a K_{D} of less than about 20 nM, less than about 10 nM, less than about 8 nM, less than about 7 nM, less than about 6 nM, less than about 5 nM, less than about 4 nM, less than about 3 nM, less than about 2 nM, less than about 1 nM, less than about 800 pM, less than about 700 pM, less than about 500 pM, less than about 400 pM, less than about 300 pM, less than about 200 pM, less than about 100 pM, less than about 50 pM, or less than about 25 pM as measured by surface plasmon resonance, e.g., using an assay format as defined in Example 2 herein, or a substantially similar assay. The present disclosure includes bispecific antigen-binding molecules (e.g., bispecific antibodies which bind human GPRC5D with a K_{D} of less than about 25 pM, and which bind monkey GPRC5D with a K_{D} of less than about 170 pM, as measured by surface plasmon resonance, e.g., using an assay format as defined in Example 2 herein, or a substantially similar assay.

The present disclosure also includes antibodies and antigen-binding fragments thereof that bind GPRC5D with a dissociative half-life (t1/2) of greater than about 10 minutes or greater than about 125 minutes as measured by surface plasmon resonance at 25° C., e.g., using an assay format as defined in Example 2 herein, or a substantially similar assay. In certain embodiments, the antibodies or antigen-binding fragments of the present disclosure bind GPRC5D with a t1/2 of greater than about 3 minutes, greater than about 4 minutes, greater than about 10 minutes, greater than about 20 minutes, greater than about 30 minutes, greater than about 40 minutes, greater than about 50 minutes, greater than about 60 minutes, greater than about 70 minutes, greater than about 80 minutes, greater than about 90 minutes, greater than about 100 minutes, greater than about 110 minutes, or greater than about 120 minutes, as measured by surface plasmon resonance at 25° C., e.g., using an assay format as defined in Example 2 herein, or a substantially similar assay. The present disclosure includes bispecific antigen-binding molecules (e.g., bispecific antibodies which bind GPRC5D with a of greater than about 10 minutes as measured by surface plasmon resonance at 25° C., e.g., using an assay format as defined in Example 2 herein, or a substantially similar assay.

The present disclosure also includes antibodies and antigen-binding fragments thereof which bind specifically to human cell lines which express endogenous GPRC5D (e.g., NCI-H929, MOLP-8 or OMP-2), as determined by a FACS binding assay as set forth in Example 3 or a substantially similar assay. The expression of GPRC5D varies from one cell line to another, with MOLP-8 expressing a low number of surface available GPRC5D proteins (˜4400), H929 expressing a moderate number of surface exposed GPRC5D proteins (˜9200), and OPM-2 expressing a high number of surface exposed GPRC5D proteins (˜11700).

The present disclosure also includes anti-GPRC5D x anti-CD28 bispecific antigen-binding molecules which exhibit tumor inhibition in mice bearing human multiple myeloma xenografts of MOLP-8, H929, and OPM-2 cells alone or in combination with an anti-BCMA x CD3 bispecific antibody.

The present disclosure includes antibodies and antigen-binding fragments thereof that bind human CD28 with high affinity. The present disclosure also includes antibodies and antigen-binding fragments thereof that bind human CD28 with medium or low affinity, depending on the therapeutic context and particular targeting properties that are desired. For example, in the context of a bispecific antigen-binding molecule, wherein one arm binds CD28 and another arm binds a target antigen (e.g., GPRC5D), it may be desirable for the target antigen-binding arm to bind the target antigen with high affinity while the anti-CD28 arm binds CD28 with only moderate or low affinity or no affinity. In this manner, preferential targeting of the antigen-binding molecule to cells expressing the target antigen may be achieved while avoiding general/untargeted CD28 binding and the consequent adverse side effects associated therewith. In some embodiments, the bispecific antigen-binding molecules of the present disclosure are advantageous in that they can target tumor cells expressing GPRC5D without inducing unwanted off-target effects characterized by skin, nail, and taste toxicities, which have been observed in other GPRC5D-targeted immunotherapies.

The anti-CD28 antibodies of the present disclosure can be administered (e.g., to human subjects) without inducing a cytokine storm. Anti-CD28 antibodies have been proposed for therapeutic purposes involving the activation of T cells. One particular anti-CD28 antibody, TGN1412 (anti-CD28 superagonist), was used in a clinical trial in 2006. Six healthy volunteers were dosed intravenously with TGN1412 (anti-CD28 superagonist) at a dose of 0.1 mg/kg. Within two hours, all six patients had significant inflammatory responses (cytokine storm), and all patients were in multi-organ failure within sixteen hours. Subjects were treated with corticosteroids, and cytokine levels returned to normal within 2-3 days. The starting dose of 0.1 mg/kg in a Phase 1 study was based on a 500-fold multiple of the no-observed-adverse-effect-level (“NOAEL”) of 50 mg/kg in cynomolgus monkeys (Suntharalingam, et al., Cytokine Storm in a Phase 1 Trial of the Anti-CD28 Monoclonal Antibody TGN1412, NEJM 355:1018-1028 (2006)). Unfortunately, the cytokine storm induced by TGN1412 was not predicted by toxicology studies in cynomolgus macaques or in ex vivo human PBMC studies.

The present disclosure includes bispecific antigen-binding molecules (e.g., bispecific antibodies) which are capable of simultaneously binding to human CD28 and a human GPRC5D. The binding arm that interacts with cells that express CD28 may have high binding or weak binding as measured in a suitable in vitro binding assay. The extent to which a bispecific antigen-binding molecule binds cells that express CD28 and/or GPRC5D can be assessed by fluorescence activated cell sorting (FACS), as illustrated in Example 3 herein. For example, the present disclosure includes antibodies, antigen-binding fragments, and bispecific antibodies thereof which specifically bind human T-cell lines which express CD28 but do not express GPRC5D (e.g., Jurkat), and/or GPRC5D-expressing cells. The present disclosure also includes antibodies, antigen-binding fragments, and bispecific antibodies thereof that bind human CD28 and induce T cell activation.

### Epitope Mapping and Related Technologies

The epitope on CD28 and/or GPRC5D to which the antigen-binding molecules of the present disclosure bind may consist of a single contiguous sequence of 3 or more (e.g., 3, 4, 5, 6, 7, 8, 9, 10, 11, 12, 13, 14, 15, 16, 17, 18, 19, 20 or more) amino acids of a CD28 or GPRC5D protein. Alternatively, the epitope may consist of a plurality of non-contiguous amino acids (or amino acid sequences) of CD28 or GPRC5D. The term “epitope,” as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sulfonyl groups on the antigen.

Various techniques known to persons of ordinary skill in the art can be used to determine whether an antigen-binding domain of an antibody “interacts with one or more amino acids” within a polypeptide or protein. Exemplary techniques include, e.g., routine cross-blocking assay such as that described Antibodies, Harlow and Lane (Cold Spring Harbor Press, Cold Spring Harb., NY), alanine scanning mutational analysis, peptide blots analysis (Reineke, 2004, Methods Mol Biol 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antigen-binding domain of an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73: 256A-265A. X-ray crystallography of the antigen/antibody complex may also be used for epitope mapping purposes.

The present disclosure further includes anti-GPRC5D antibodies that bind to the same epitope as any of the specific exemplary antibodies described herein (e.g. antibodies comprising any of the amino acid sequences as set forth in Table 1A herein). Likewise, the present disclosure also includes anti-GPRC5D antibodies that compete for binding to GPRC5D with any of the specific exemplary antibodies described herein (e.g. antibodies comprising any of the amino acid sequences as set forth in Table 1A herein).

Likewise, the present disclosure also includes bispecific antigen-binding molecules comprising a first antigen-binding domain that specifically binds human GPRC5D, and a second antigen binding domain that specifically binds human CD28, wherein the first antigen-binding domain competes for binding to GPRC5D with any of the specific exemplary GPRC5D-specific antigen-binding domains described herein, and/or wherein the second antigen-binding domain competes for binding to CD28 with any of the specific exemplary CD28-specific antigen-binding domains described herein.

One can easily determine whether a particular antigen-binding molecule (e.g., antibody) or antigen-binding domain thereof binds to the same epitope as, or competes for binding with, a reference antigen-binding molecule of the present disclosure by using routine methods known in the art. For example, to determine if a test antibody binds to the same epitope on GPRC5D (or CD28) as a reference bispecific antigen-binding molecule of the present disclosure, the reference bispecific molecule is first allowed to bind to a GPRC5D protein (or CD28 protein). Next, the ability of a test antibody to bind to the GPRC5D (or CD28) molecule is assessed. If the test antibody is able to bind to GPRC5D (or CD28) following saturation binding with the reference bispecific antigen-binding molecule, it can be concluded that the test antibody binds to a different epitope of GPRC5D (or CD28) than the reference bispecific antigen-binding molecule. On the other hand, if the test antibody is not able to bind to the GPRC5D (or CD28) molecule following saturation binding with the reference bispecific antigen-binding molecule, then the test antibody may bind to the same epitope of GPRC5D (or CD28) as the epitope bound by the reference bispecific antigen-binding molecule of the disclosure. Additional routine experimentation (e.g., peptide mutation and binding analyses) can then be carried out to confirm whether the observed lack of binding of the test antibody is in fact due to binding to the same epitope as the reference bispecific antigen-binding molecule or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present disclosure, two antigen-binding proteins bind to the same (or overlapping) epitope if, e.g., a 1-, 5-, 10-, 20- or 100-fold excess of one antigen-binding protein inhibits binding of the other by at least 50% but preferably 75%, 90% or even 99% as measured in a competitive binding assay (see, e.g., Junghans et al., Cancer Res. 1990:50:1495-1502). Alternatively, two antigen-binding proteins are deemed to bind to the same epitope if essentially all amino acid mutations in the antigen that reduce or eliminate binding of one antigen-binding protein reduce or eliminate binding of the other. Two antigen-binding proteins are deemed to have “overlapping epitopes” if only a subset of the amino acid mutations that reduce or eliminate binding of one antigen-binding protein reduce or eliminate binding of the other.

To determine if an antibody or antigen-binding domain thereof competes for binding with a reference antigen-binding molecule, the above-described binding methodology is performed in two orientations: In a first orientation, the reference antigen-binding molecule is allowed to bind to a GPRC5D protein (or CD28 protein) under saturating conditions followed by assessment of binding of the test antibody to the GPRC5D (or CD28) molecule. In a second orientation, the test antibody is allowed to bind to a GPRC5D (or CD28) molecule under saturating conditions followed by assessment of binding of the reference antigen-binding molecule to the GPRC5D (or CD28) molecule. If, in both orientations, only the first (saturating) antigen-binding molecule is capable of binding to the GPRC5D (or CD28) molecule, then it is concluded that the test antibody and the reference antigen-binding molecule compete for binding to GPRC5D (or CD28). As will be appreciated by a person of ordinary skill in the art, an antibody that competes for binding with a reference antigen-binding molecule may not necessarily bind to the same epitope as the reference antibody, but may sterically block binding of the reference antibody by binding an overlapping or adjacent epitope.

The anti-GPRC5D antibodies or antigen-binding molecules of the present disclosure may bind to the extracellular loop (ECL) domains 2 and 3 of the GPRC5D protein, rather than ECL1 or the N-terminus of the protein. In some cases, the anti-GPRC5D antibodies or antigen-binding molecules of the present disclosure preferentially bind ECL2 and ECL3 of GPRC5D, relative to ECL1 and the N-terminus of GPRC5D.

### Polynucleotides, Vectors, and Host Cells

In various embodiments, the present disclosure provides a polynucleotide encoding a polypeptide (e.g., an antibody or part thereof) discussed herein, a vector comprising the polynucleotide, and/or a host cell comprising the antibody or antigen-binding fragment or polypeptide or polynucleotide or vector. In various embodiments, the present disclosure provides a polynucleotide that encodes a HCVR, a LCVR, or both a HCVR and a LCVR of an antibody or antigen-binding fragment thereof as discussed herein. The HCVR and/or LCVR may be defined by the CDRs contained within the HCVR sequence, the LCVR sequence, or both the HCVR sequence and the LCVR sequence, respectively, as set forth in Table 1A or Table 2 for the amino acid sequences and Table 1B or Table 3 for the nucleic acid sequences. The HCVR and/or LCVR may also be defined by the heavy chain CDR sequences, the light chain CDR sequences, or both the heavy and light chain CDR sequences, respectively, as set forth in Table 1A or Table 2 for the amino acid sequences and Table 1B or Table 3 for the nucleic acid sequences. The HCVR and/or LCVR may also be defined by the HCVR sequence, the LCVR sequence, or both the HCVR and LCVR sequences, respectively, as set forth in Table 1A or Table 2 for the amino acid sequences and Table 1B or Table 3 for the nucleic acid sequences. In various embodiments, the present disclosure provides a polynucleotide that encodes a heavy chain, a light chain, or both a heavy chain and a light chain of an antibody as discussed above or herein. The heavy chain and/or light chain may be defined by the heavy and light chain sequences, respectively, as set forth in Table 4. In various embodiments, the polynucleotide comprises a nucleic acid sequence as set forth in Table 4. In various embodiments, the present disclosure provides a vector or vectors comprising the polynucleotides discussed above, and/or a host cell comprising the polynucleotides or vectors, or HCVR or LCVR, of HC or LC, or an assembled antibody or antigen-binding fragment thereof as discussed above or herein. In various embodiments, the present disclosure provides a pair of polynucleotides, wherein (a) the first polynucleotide encodes: (i) a HCVR comprising the CDR sequences contained in a HCVR of an antibody of Table 1A or Table 2, (ii) a HCVR comprising the HCDR1, HCDR2 and HCDR3 sequences as set forth for an antibody in Table 1A or Table 2, (iii) a HCVR comprising the HCVR sequence of an antibody of Table 1A or Table 2, or (iv) a heavy chain (HC) comprising the HC sequence of an antibody of Table 4, and (b) the second polynucleotide encodes: (i) a LCVR comprising the CDR sequences contained in a LCVR of an antibody of Table 1A or Table 2, (ii) a LCVR comprising the LCDR1, LCDR2 and LCDR3 sequences as set forth for an antibody in Table 1A or Table 2, (iii) a LCVR comprising the LCVR sequence of an antibody of Table 1A or Table 2, or (iv) a light chain (LC) comprising the LC sequence of an antibody of Table 4. In various embodiments, the present disclosure descriptions of vectors comprising, respectively, the polynucleotides discussed above, and/or a host cell comprising the vectors. A host cell can be, for example, a hamster ovary cell. In any of the various embodiments, the nucleic acid sequence for the corresponding component of the antibody or antigen-binding fragment thereof (e.g., the HCVR, the LCVR, or any one or all of the CDRs) may be as set forth in Table 1B, Table 3, or Table 4.

In various embodiments, the present disclosure provides a group of nucleic acid molecules comprising nucleotide sequences, respectively, encoding the heavy chain or the HCVR or the HCDRs of the first antigen-binding domain, the heavy chain or the HCVR or the HCDRs of the second antigen-binding domain, and the light chain or the LCVR, or the LCDRs of the first and second antigen-binding domains of a bispecific antigen-binding molecule. In various embodiments, the present disclosure provides a group of polynucleotides encoding a bispecific antigen-binding molecule (e.g., a bispecific antibody), wherein (a) the first polynucleotide encodes: (i) a first HCVR comprising the CDR sequences contained in a HCVR of an antibody of Table 3, (ii) a first HCVR comprising the HCDR1, HCDR2 and HCDR3 sequences as set forth for an antibody in Table 3, (iii) a first HCVR comprising the HCVR sequence of an antibody of Table 3, or (iv) a first heavy chain (HC) comprising the HC sequence of an antibody of Table 3, (b) the second polynucleotide encodes: (i) a second HCVR comprising the CDR sequences contained in a HCVR of an antibody of Table 3, (ii) a second HCVR comprising the HCDR1, HCDR2 and HCDR3 sequences as set forth for an antibody in Table 3, (iii) a second HCVR comprising the HCVR sequence of an antibody of Table 3, or (iv) a second heavy chain (HC) comprising the HC sequence of an antibody of Table 3, and (c) the third polynucleotide encodes: (i) a common LCVR comprising the CDR sequences contained in a LCVR of an antibody of Table 3, (ii) a common LCVR comprising the LCDR1, LCDR2 and LCDR3 sequences as set forth for an antibody in Table 3, (iii) a common LCVR comprising the LCVR sequence of an antibody of Table 3, or (iv) a common light chain (LC) comprising the LC sequence of an antibody of Table 3. In any of the various embodiments, the nucleic acid sequence for the corresponding component of the antibody or antigen-binding fragment thereof (e.g., the HCVR, the LCVR, or any one or all of the CDRs) may be as set forth in Table 4. The present disclosure also includes a group of expression vectors comprising, respectively, the group of nucleic acid molecules, as discussed above or herein, and a host cell comprising a bispecific antigen-binding molecule as discussed above or herein, a group of nucleic acid molecules as discussed above or herein, or a group of expression vectors as discussed above or herein.

### Preparation of Antigen-Binding Domains and Construction of Bispecific Molecules

Antigen-binding domains specific for particular antigens can be prepared by any antibody generating technology known in the art. Once obtained, two different antigen-binding domains, specific for two different antigens (e.g., CD28 and GPRC5D), can be appropriately arranged relative to one another to produce a bispecific antigen-binding molecule of the present disclosure using routine methods. In certain embodiments, one or more of the individual components (e.g., heavy and light chains) of the multispecific antigen-binding molecules of the disclosure are derived from chimeric, humanized or fully human antibodies. Methods for making such antibodies are well known in the art. For example, one or more of the heavy and/or light chains of the bispecific antigen-binding molecules of the present disclosure can be prepared using VELOCIMMUNE™ technology. Using VELOCIMMUNE™ technology (or any other human antibody generating technology), high affinity chimeric antibodies to a particular antigen (e.g., CD28 or GPRC5D) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules of the present disclosure.

Genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human bispecific antigen-binding molecules comprising two different heavy chains that associate with an identical light chain that comprises a variable domain derived from one of two different human light chain variable region gene segments. (See, e.g., US 2011/0195454). Fully human refers to an antibody, or antigen-binding fragment or immunoglobulin domain thereof, comprising an amino acid sequence encoded by a DNA derived from a human sequence over the entire length of each polypeptide of the antibody or antigen-binding fragment or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g., compared to any wild-type human immunoglobulin regions or domains.

### BIOEQUIVALENTS

The present disclosure encompasses antigen-binding molecules having amino acid sequences that vary from those of the exemplary molecules disclosed herein but that retain the ability to bind CD28 and/or GPRC5D. Such variant molecules may comprise one or more additions, deletions, or substitutions of amino acids when compared to parent sequence, but exhibit biological activity that is essentially equivalent to that of the described bispecific antigen-binding molecules.

The present disclosure includes antigen-binding molecules that are bioequivalent to any of the exemplary antigen-binding molecules set forth herein. Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antigen-binding proteins will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding proteins may include variants of the exemplary bispecific antigen-binding molecules set forth herein comprising amino acid changes which modify the glycosylation characteristics of the molecules, e.g., mutations which eliminate or remove glycosylation.

### Species Selectivity and Species Cross-Reactivity

According to certain embodiments of the disclosure, antigen-binding molecules are provided which bind to human CD28 but not to CD28 from other species. Also provided are antigen-binding molecules which bind to human GPRC5D, but not to GPRC5D from other species. The present disclosure also includes antigen-binding molecules that bind to human CD28 and to CD28 from one or more non-human species; and/or antigen-binding molecules that bind to human GPRC5D and to GPRC5D from one or more non-human species.

According to certain exemplary embodiments of the disclosure, antigen-binding molecules are provided which bind to human CD28 and/or human GPRC5D and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee CD28 and/or GPRC5D. For example, in particular exemplary embodiments of the present disclosure bispecific antigen-binding molecules are provided comprising a first antigen-binding domain that binds human GPRC5D and cynomolgus GPRC5D, and a second antigen-binding domain that specifically binds human CD28, or bispecific antigen-binding molecules comprising a first antigen-binding domain that binds human GPRC5D and cynomolgus GPRC5D, and a second antigen-binding domain that specifically binds human CD28.

### Therapeutic Uses of the Antigen-Binding Molecules

The present disclosure includes methods comprising administering to a subject in need thereof a therapeutic composition comprising an anti-GPRC5D antibody or antigen-binding fragment thereof, or a bispecific antigen-binding molecule that specifically binds CD28 and GPRC5D. The therapeutic composition may comprise any of the antibodies or bispecific antigen-binding molecules as disclosed herein and a pharmaceutically acceptable carrier or diluent. As used herein, the expression “a subject in need thereof” means a human or non-human animal that exhibits one or more symptoms or indicia of cancer (e.g., a subject having a GPRC5D-expressing tumor or suffering from any of the cancers mentioned herein), or who otherwise would benefit from an inhibition or reduction in GPRC5D activity or a depletion of GPRC5D+ cells (e.g., multiple myeloma cells).

The antibodies and bispecific antigen-binding molecules of the disclosure (and therapeutic compositions comprising the same) are useful, inter alia, for treating any disease or disorder in which stimulation, activation and/or targeting of an immune response would be beneficial. In particular, the anti-GPRC5D antibodies or the anti-GPRC5D x anti-CD28 bispecific antigen-binding molecules of the present disclosure may be used for the treatment, prevention and/or amelioration of any disease or disorder associated with or mediated by GPRC5D expression or activity or the proliferation of GPRC5D+ cells. The mechanism of action by which the therapeutic methods of the disclosure are achieved include killing of the cells expressing GPRC5D in the presence of effector cells, for example, by CDC, apoptosis, ADCC, phagocytosis, or by a combination of two or more of these mechanisms. Cells expressing GPRC5D that can be inhibited or killed using the bispecific antigen-binding molecules of the disclosure include, for example, multiple myeloma cells. In certain embodiments, the GPRC5D-expressing cancer is multiple myeloma, non-small-cell lung cancer, head & neck cancer, breast cancer, colorectal cancer, pancreatic cancer, ovarian cancer, non-Hodgkin's lymphoma, renal cell carcinoma, lung cancer, liver cancer, or stomach cancer.

According to certain embodiments of the present disclosure, the anti-GPRC5D antibodies or anti-GPRC5D x anti-CD28 bispecific antigen-binding molecules (e.g., a bispecific antibody) are useful for treating a patient afflicted with multiple myeloma. Analytic/diagnostic methods known in the art, such as tumor scanning, etc., may be used to ascertain whether a patient harbors multiple myeloma or another GPRC5D-expressing cancer. The present disclosure also includes methods for treating residual cancer in a subject. As used herein, the term “residual cancer” means the existence or persistence of one or more cancerous cells in a subject following treatment with an anti-cancer therapy.

According to certain aspects, the present disclosure provides methods for treating a disease or disorder associated with GPRC5D expression (e.g., multiple myeloma) comprising administering one or more of the anti-GPRC5D antibodies (or antigen-binding fragments thereof) or bispecific antigen-binding molecules described elsewhere herein to a subject after the subject has been determined to have multiple myeloma. For example, the present disclosure includes methods for treating multiple myeloma comprising administering an anti-GPRC5D antibody or antigen-binding fragment thereof, or an anti-GPRC5D x anti-CD28 bispecific antigen-binding molecule (e.g., a bispecific antibody) to a patient 1 day, 2 days, 3 days, 4 days, 5 days, 6 days, 1 week, 2 weeks, 3 weeks or 4 weeks, 2 months, 4 months, 6 months, 8 months, 1 year, or more after the subject has received other immunotherapy or chemotherapy.

The present disclosure also includes methods of treating lymphoproliferative diseases resulting from abnormal proliferation of plasma cells. The lymphoproliferative diseases include multiple myeloma (MM), including both asymptomatic and symptomatic stages of the disease.

In some cases, the present disclosure is directed to methods of treating symptomatic multiple myeloma in newly-diagnosed or treatment-naïve subjects. The treatment-naïve subjects may have received prior radiation therapy or up to one month of corticosteroid therapy to treat or manage symptoms of multiple myeloma, but have not otherwise received any systemic therapy for the treatment of multiple myeloma.

In some cases, the present disclosure is directed to methods of reducing the risk of developing a symptomatic lymphoproliferative disease (e.g., multiple myeloma). Monoclonal gammopathy of undetermined significance (MGUS) is reportedly associated with an approximate 1% risk, per year, of progression to symptomatic multiple myeloma, Waldenstrom's macroglobulinemia, or other lymphoproliferative disorders. Similarly, smoldering multiple myeloma (SMM) is reportedly associated with an approximate 10% risk, per year (for the first five years), an approximate 3% risk, per year (for the next five years), and an approximate 1-2% risk, per year (for the following ten years), of progression to symptomatic multiple myeloma (see Kyle et al., Leukemia, 24 (6): 1121-1127, 2010). The methods discussed herein reduce the risk of developing symptomatic multiple myeloma (or other lymphoproliferative disease of plasma cell origin) in subject diagnosed with MGUS or SMM relative to a control population that has not received the bispecific antibody therapy discussed herein, or the reported risk discussed above.

As discussed herein, MGUS is associated with a serum monoclonal protein (M protein) level of <3 g/dL, clonal bone marrow plasma cells of <10%, and an absence of end-organ damage such as hypercalcemia, renal insufficiency, anemia and bone lesions that can be attributed to the plasma cell proliferative disorder. SMM is associated with a serum monoclonal protein (IgG or IgA)≥3 g/dL and/or clonal bone marrow plasma cells of ≥10%, and an absence of end-organ damage such as hypercalcemia, renal insufficiency, anemia and bone lesions that can be attributed to the plasma cell proliferative disorder. In contrast, multiple myeloma (symptomatic MM) is associated with clonal bone marrow plasma cells of ≥10%, presence of serum and/or urinary monoclonal protein (except in patients with non-secretory multiple myeloma), and evidence of end organ damage that can be attributed to the underlying plasma cell proliferative disorder, specifically (a) hypercalcemia, wherein serum calcium is >11 mg/dL; (b) renal insufficiency, wherein serum creatinine is >177 μmol/L; (c) anemia with a hemoglobin value ≥2 g/dL below the lower limit of normal, or a hemoglobin value of <10 g/dL; (d) more than one myeloma-related lesion on advanced imaging, including magnetic resonance imaging (MRI), positron emission tomography/computed tomography (PET/CT), or whole-body low dose computed tomography (WBLDCT); (e) clonal bone marrow plasma cells ≥60%; or (f) involved/uninvolved serum free light chain (FLC) ratio of 100 or more.

In some cases, specific predictors of enhanced risk of progression from asymptomatic to symptomatic multiple myeloma can be used to identify patients for intervention with the bispecific antibody therapy discussed herein. In particular, the size the M protein, the type of M protein, the number of bone marrow plasma cells, and the free light chain (FLC) ratio may be used to identify patients with an increased risk of progression to symptomatic disease. In some cases, an M protein level of ≥5 g/L, ≥15 g/L, or ≥25 g/L can be used to identify patients with an increasing risk of progression from asymptomatic to symptomatic disease. In some cases, presence of an IgM or IgA monoclonal protein (as opposed to IgG) can be used to identify patients with an increased risk of progression from asymptomatic to symptomatic disease. In some cases, presence of >5% clonal bone marrow plasma cells can be used to identify patients with an increased risk of progression from asymptomatic to symptomatic disease. In some cases, an abnormal FLC ratio can be used to identify patients with an increased risk of progression from asymptomatic to symptomatic disease. In some cases, any two or more of these factors can be combined to identify patients at increased risk of progression from asymptomatic to symptomatic multiple myeloma. Thus, in some cases, the methods discussed herein include identifying a subject with one or more of the factors discussed above, and/or measuring one or more of the factors discussed above. In some cases, the methods discussed herein include administration of the bispecific antibody (or antigen-binding fragment thereof) to a subject identified as having one or more of the factors discussed above.

The present disclosure includes methods comprising administering to a subject in need thereof a therapeutic composition comprising an anti-GPRC5D x anti-CD28 bispecific antibody or antigen-binding fragment thereof. The therapeutic composition can comprise any of the bispecific antigen-binding molecules as disclosed herein and a pharmaceutically acceptable carrier or diluent. As used herein, the expression “a subject in need thereof” means a human or non-human animal that exhibits one or more indicia of an asymptomatic or symptomatic lymphoproliferative disorder (e.g., multiple myeloma) as discussed herein.

The present disclosure also includes methods for treating relapsed or refractory lymphoproliferative disorders (e.g., multiple myeloma) in a subject. In some cases, the subject may have been previously treated with an anti-CD38 antibody therapy. In some cases, the anti-CD38 antibody is daratumumab or isatuximab. In some cases, the subject may have been previously treated with a proteasome inhibitor or an immunomodulatory drug. In some cases, the proteasome inhibitor is bortezomib, carfilzomib or ixazomib. In some cases, the immunomodulatory drug is lenalidomide or pomalidomide. In some cases, the subject has relapsed or refractory multiple myeloma following one or more (e.g., two or more, three or more, four or more, or five or more) prior systemic treatments, including any one or more of the previous treatments discussed above or herein. In some cases, the subject is at least triple-refractory to prior therapies (i.e., progressed after at least three prior lines of therapy). In some cases, the subject is quad-refractory to prior therapies. In some cases, the subject is penta-refractory to prior therapies.

In some cases, administration of the bispecific antibody therapy discussed herein results in a stringent complete response, a complete response, a very good partial response, or a partial response. In some cases, a stringent complete response (sCR) corresponds to negative immunofixation on the serum and urine and disappearance of any soft tissue plasmacytomas and <5% plasma cells in bone marrow aspirates, plus normal FLC ratio and absence of clonal cells in bone marrow biopsy by immunohistochemistry (κ/λratio≤4:1 or ≥1:2 for κ and λ patients, respectively, after counting ≥100 plasma cells). In some cases, a complete response (CR) corresponds to negative immunofixation on the serum and urine and disappearance of any soft tissue plasmacytomas and <5% plasma cells in bone marrow aspirates. In some cases, a very good partial response (VGPR) corresponds to serum and urine M-protein detectable by immunofixation but not on electrophoresis or ≥90% reduction in serum M-protein plus urine M-protein level <100 mg per 24 h. In some cases, a partial response (PR) corresponds to ≥50% reduction of serum M-protein plus reduction in 24 h urinary M-protein by ≥90% or to <200 mg per 24 h; If the serum and urine M-protein are unmeasurable, a ≥50% decrease in the difference between involved and uninvolved FLC levels is required in place of the M-protein criteria; If serum and urine M-protein are unmeasurable, and serum-free light assay is also unmeasurable, ≥50% reduction in plasma cells is required in place of M-protein, provided baseline bone marrow plasma-cell percentage was ≥30%. In addition to these criteria, if present at baseline, a ≥50% reduction in the size of soft tissue plasmacytomas is also required.

### Combination Therapies and Formulations

The present disclosure provides methods which comprise administering a pharmaceutical composition comprising any of the exemplary antibodies and bispecific antigen-binding molecules described herein in combination with one or more additional therapeutic agents. Exemplary additional therapeutic agents that may be combined with or administered in combination with an antigen-binding molecule of the present disclosure include, e.g., an anti-tumor agent (e.g. chemotherapeutic agents including melphalan, vincristine (Oncovin), cyclophosphamide (Cytoxan), etoposide (VP-16), doxorubicin (Adriamycin), liposomal doxorubicin (Doxil), bendamustine (Treanda), or any others known to be effective in treating a plasma cell tumor in a subject). In some embodiments, the second therapeutic agent comprises steroids. In some embodiments, the second therapeutic agent comprises largeled therapies including thalidomide, lenalidomide, and bortezomib, which are therapies approved to treat newly diagnosed patients. Lenalidomide, pomalidomide, bortezomib, carfilzomib, panobinostat, ixazomib, elotuzumab, and daratumumab are examples of a second therapeutic agent effective for treating recurrent myeloma. In certain embodiments the second therapeutic agent is a regimen comprising radiotherapy or a stem cell transplant. In certain embodiments, the second therapeutic agent may be an immunomodulatory agent. In certain embodiments, the second therapeutic agent may be a proteasome inhibitor, including bortezomib (Velcade), carfilzomib (Kyprolis), ixazomib (Ninlaro). In certain embodiments the second therapeutic agent may be a histone deacetylase inhibitor such as panobinostat (Farydak). In certain embodiments, the second therapeutic agent may be a monoclonal antibody, an antibody drug conjugate, a bispecific antibody conjugated to an anti-tumor agent, a checkpoint inhibitor, or combinations thereof. The pharmaceutical compositions of the present disclosure (e.g., pharmaceutical compositions comprising an anti-GPRC5D x anti-CD28 bispecific antigen-binding molecule as disclosed herein) may also be administered as part of a therapeutic regimen comprising one or more therapeutic combinations selected from a monoclonal antibody other than those described herein, which may interact with a different antigen on the plasma cell surface (e.g., CD38), a bispecific antibody that has one arm that binds to an antigen on the tumor cell surface and the other arm binds to an antigen on a T cell, an antibody drug conjugate, a bispecific antibody that binds human BCMA and human CD3, a checkpoint inhibitor, for example, one that targets, PD-1, PD-L1, LAG-3 or CTLA-4, or combinations thereof. In certain embodiments, the checkpoint inhibitors may be selected from PD-1 inhibitors, such as pembrolizumab (Keytruda), nivolumab (Opdivo), or cemiplimab. In certain embodiments, the checkpoint inhibitors may be selected from PD-L1 inhibitors, such as atezolizumab (Tecentriq), avelumab (Bavencio), or Durvalumab (Imfinzi)). In certain embodiments, the checkpoint inhibitors may be selected from CTLA-4 inhibitors, such as ipilimumab (Yervoy). In some cases, the additional therapeutic agent is a bispecific anti-BCMA x CD3 antibody, for example, teclistamab, elranatamab, alnuctamab, or linvoseltamab (also referred to as REGN5458). Other bispecific anti-BCMA x CD3 antibodies that may be used in combination with the anti-GPRC5D x CD28 antibodies discussed herein (e.g., for treating a GPRC5D-expressing cancer or multiple myeloma), are those anti-BCMA x CD3 antibodies discussed in WO 2020/018820 (including REGN5458 and REGN5459).

The additional therapeutically active component(s) may be administered just prior to, concurrent with, or shortly after the administration of an antigen-binding molecule of the present disclosure; (for purposes of the present disclosure, such administration regimens are considered the administration of an antigen-binding molecule “in combination with” an additional therapeutically active component).

The present disclosure includes pharmaceutical compositions comprising an antigen-binding molecule (e.g., an anti-GPRC5D antibody or antigen-binding fragment thereof, or a bispecific antigen-binding molecule or bispecific antibody that specifically binds GPRC5D and CD28). The present disclosure also includes pharmaceutical compositions in which an antigen-binding molecule of the present disclosure is separately formulated or co-formulated with one or more of the additional therapeutically active component(s) as described elsewhere herein. The pharmaceutical compositions of the disclosure may be formulated with suitable carriers, excipients, and other agents. A multitude of appropriate formulations can be found in the formulary known to all pharmaceutical chemists: Remington's Pharmaceutical Sciences, Mack Publishing Company, Easton, PA.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded. Numerous reusable pen and autoinjector delivery devices known in the art have applications in the subcutaneous delivery of a pharmaceutical composition of the present disclosure.

### Diagnostic Uses of the Antibodies

The anti-GPRC5D antibodies of the present disclosure may also be used to detect and/or measure GPRC5D, or GPRC5D-expressing cells in a sample, e.g., for diagnostic purposes. For example, an anti-GPRC5D antibody, or fragment thereof, may be used to diagnose a condition or disease characterized by aberrant expression (e.g., over-expression, under-expression, lack of expression, etc.) of GPRC5D. Exemplary diagnostic assays for GPRC5D may comprise, e.g., contacting a sample, obtained from a patient, with an anti-GPRC5D antibody of the disclosure, wherein the anti-GPRC5D antibody is labeled with a detectable label or reporter molecule. Alternatively, an unlabeled anti-GPRC5D antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Another exemplary diagnostic use of the anti-GPRC5D antibodies of the disclosure includes ⁸⁹Zr-labeled, such as ⁸⁹Zr-desferrioxamine-labeled, antibody for the purpose of noninvasive identification and tracking of tumor cells in a subject (e.g. positron emission tomography (PET) imaging). (See, e.g., Tavare, R. et al. Cancer Res. 2016 Jan. 1; 76(1):73-82; and Azad, B B. et al. Oncotarget. 2016 Mar. 15; 7(11):12344-58.) Specific exemplary assays that can be used to detect or measure GPRC5D in a sample include enzyme-linked immunosorbent assay (ELISA), radioimmunoassay (RIA), and fluorescence-activated cell sorting (FACS).

Samples that can be used in GPRC5D diagnostic assays according to the present disclosure include any tissue or fluid sample obtainable from a patient which contains detectable quantities of GPRC5D protein, or fragments thereof, under normal or pathological conditions. Generally, levels of GPRC5D in a particular sample obtained from a healthy patient (e.g., a patient not afflicted with a disease or condition associated with abnormal GPRC5D levels or activity) will be measured to initially establish a baseline, or standard, level of GPRC5D. This baseline level of GPRC5D can then be compared against the levels of GPRC5D measured in samples obtained from individuals suspected of having a GPRC5D related disease (e.g., a tumor containing GPRC5D-expressing cells) or condition.

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Generation of Anti-GPRC5D Antibodies and Bispecific Anti-GPRC5D x CD28 Antibodies

Anti-GPRC5D antibodies were obtained by immunizing an engineered mouse comprising DNA encoding human immunoglobulin heavy and light (ULC3-20) chain variable regions with a human GPRC5D full-length DNA and full-length GPRC5D protein. Following immunization, splenocytes were harvested from each mouse and either (1) fused with mouse myeloma cells to preserve their viability and form hybridoma cells and screened for GPRC5D specificity, or (2) B-cell sorted (as described in US 2007/0280945A1) using human GPRC5D protein as the sorting reagent that binds and identifies reactive antibodies (antigen-positive B cells).

Chimeric antibodies to GPRC5D were initially isolated having a human variable region and a mouse constant region. The antibodies were characterized and selected for desirable characteristics, including affinity, selectivity, etc. If necessary, mouse constant regions were replaced with a desired human constant region, for example wild-type or modified IgG1 or IgG4 constant region, to generate a fully human anti-GPRC5D antibody. While the constant region selected may vary according to specific use, high affinity antigen-binding and target specificity characteristics reside in the variable region.

Heavy and Light Chain Variable Region Amino Acid and Nucleic Acid Sequences of anti-GPRC5D antibodies: Table 1A sets forth the amino acid sequence identifiers of the heavy and light chain variable regions and CDRs of selected anti-GPRC5D antibodies of the disclosure. The corresponding nucleic acid sequence identifiers are set forth in Table 1B.

TABLE 1A
Amino Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb77036 | 2 | 4 | 6 | 8 | 18 | 20 | 22 | 24 |
| mAb77056 | 32 | 34 | 36 | 38 | 18 | 20 | 22 | 24 |
| mAb77083 | 42 | 12 | 44 | 46 | 18 | 20 | 22 | 24 |

TABLE 1B
Nucleic Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb77036 | 1 | 3 | 5 | 7 | 17 | 19 | 21 | 23 |
| mAb77056 | 31 | 33 | 35 | 37 | 17 | 19 | 21 | 23 |
| mAb77083 | 41 | 11 | 43 | 45 | 17 | 19 | 21 | 23 |

The present disclosure also provides bispecific antigen-binding molecules that bind GPRC5D and CD28; such bispecific antigen-binding molecules are also referred to herein as “anti-GPRC5D x anti-CD28 or anti-GPRC5D x CD28 or anti-CD28 x GPRC5D bispecific molecules or bispecific antibodies.” The anti-GPRC5D portion of the anti-GPRC5D x anti-CD28 bispecific molecules is useful for targeting tumor cells that express GPRC5D (G protein-coupled receptor, class C, group 5, member D), and the anti-CD28 portion of the bispecific molecules is useful for activating T-cells. The simultaneous binding of GPRC5D on a tumor cell and CD28 on a T-cell facilitates directed killing (cell lysis) of the targeted tumor cell by the activated T-cell.

Bispecific antibodies comprising an anti-GPRC5D-specific binding domain and an anti-CD28-specific binding domain were constructed using standard methodologies, wherein the anti-GPRC5D antigen binding domain and the anti-CD28 antigen binding domain each comprise different, distinct HCVRs paired with a common LCVR. In exemplified bispecific antibodies, the molecules were constructed utilizing a heavy chain from an anti-GPRC5D antibody, a heavy chain from an anti-CD28 antibody (mAb14226; U.S. Pat. No. 11,548,947) and a common light chain (ULC3-20).

Table 2 shows the amino acid sequence identifiers for selected bispecific anti-GPRC5D x anti-CD28 antibodies exemplified herein. The corresponding nucleic acid sequence identifiers are set forth in Table 3.

TABLE 2
Amino Acid Sequences of Selected Anti-
GPRC5D × Anti-CD28 Bispecific Antibodies

| Bispecific | Anti-GPRC5D | | | | Anti-CD28 | | | |
|---|---|---|---|---|---|---|---|---|
| Antibody | Heavy Chain | | | | Heavy Chain | | | |
| Identifier | HCVR | HCDR1 | HCDR2 | HCDR3 | HCVR | HCDR1 | HCDR2 | HCDR3 |
| bsAb17372 | 2 | 4 | 6 | 8 | 10 | 12 | 14 | 16 |
| bsAb17374 | 32 | 34 | 36 | 38 | 10 | 12 | 14 | 16 |
| bsAb17375 | 42 | 12 | 44 | 46 | 10 | 12 | 14 | 16 |

Common Light Chain for all three Bispecific Antibodies

| Bispecific | | | | |
|---|---|---|---|---|
| Antibody | | | | |
| Identifier | LCVR | LCDR1 | LCDR2 | LCDR3 |
| bsAb17372 | 18 | 20 | 22 | 24 |
| bsAb17374 | | | | |
| bsAb17375 | | | | |

TABLE 3
Nucleic Acid Sequences of Selected Anti-
GPRC5D × Anti-CD28 Bispecific Antibodies

| Bispecific | Anti-GPRC5D | | | | Anti-CD28 | | | |
|---|---|---|---|---|---|---|---|---|
| Antibody | Heavy Chain | | | | Heavy Chain | | | |
| Identifier | HCVR | HCDR1 | HCDR2 | HCDR3 | HCVR | HCDR1 | HCDR2 | HCDR3 |
| bsAb17372 | 1 | 3 | 5 | 7 | 9 | 11 | 13 | 15 |
| bsAb17374 | 31 | 33 | 35 | 37 | 9 | 11 | 13 | 15 |
| bsAb17375 | 41 | 11 | 43 | 45 | 9 | 11 | 13 | 15 |

Common Light Chain for all three Bispecific Antibodies

| Bispecific | | | | |
|---|---|---|---|---|
| Antibody | | | | |
| Identifier | LCVR | LCDR1 | LCDR2 | LCDR3 |
| bsAb17372 | 17 | 19 | 21 | 23 |
| bsAb17374 | | | | |
| bsAb17375 | | | | |

Unless indicated otherwise, all bispecific GPRC5DxCD28 antibodies used in the following Examples comprise a human IgG4 isotype. In one embodiment, the IgG4 Fc domain comprises 2 or more amino acid changes as disclosed in US 2010/0331527. In one embodiment, the human IgG4 Fc comprises a serine to proline mutation in the hinge region (S108P) to promote dimer stabilization.

Bispecific antibodies bsAb17372, bsAb17374, and bsAb17375 comprising a human IgG4 Fc were designated as REGN17372, REGN17374, and REGN17375 respectively. Table 4 sets forth the nucleic acid and amino acid sequence identifiers of full-length heavy chain (HC) and light chain (LC) sequences of these antibodies.

TABLE 4
Amino acid and nucleotide sequences for full length immunoglobulin
chains of designated bispecific antibodies

| Bispecific | | | | | LC (GPRC5D & | |
|---|---|---|---|---|---|---|
| Antibody | HC (GPRC5D) | | HC (CD28) | | CD28) | |
| Designation | D | P | D | P | D | P |
| REGN17372 | 25 | 26 | 27 | 28 | 29 | 30 |
| REGN17374 | 39 | 40 | 27 | 28 | 29 | 30 |
| REGN17375 | 47 | 48 | 27 | 28 | 29 | 30 |

D = Nucleotide sequence of DNA encoding indicated sequence
P = amino acid of polypeptide for the indicated sequence
Numbers refer to SEQ ID NOs for the indicated sequence

### Controls Used in the Following Examples:

In addition to isotype controls, one or more of the following controls were used in certain experiments described in the Examples below:
Control 1: a bispecific antibody with one arm binding to CD28 and the second arm binding to an unrelated antigen;
Control 2: a bispecific antibody with one arm binding to CD3 and the second arm binding to an unrelated antigen;
Comparator 1: a human monoclonal antibody against GPRC5D and having V_{H}/V_{L} sequences of antibody “GC5B596” according to US 2018/0037651 (Janssen Biotech); and
Comparator 2: a human monoclonal antibody against GPRC5D and having V_{H}/V_{L} sequences of antibody “5F11” according to WO 2019/154890 (F. Hoffman-La Roche).

### Example 2: Biacore Binding Kinetics of Bispecific Anti-GPRC5D x CD28 Antibodies

Equilibrium dissociation constants (KD values) for purified bispecific antibodies binding to captured hCD28.mFc (SEQ ID NO: 49) were determined using a real-time surface plasmon resonance biosensor using a Biacore T-200 instrument. The CM5 Biacore sensor surface was derivatized by amine coupling with a polyclonal rabbit anti-mouse Fc antibody (Cytiva, Cat #BR100838) to capture purified human CD28 ecto domain with a C-terminal mouse Fc tag (hCD28.mFc, SEQ ID NO: 49). This Biacore binding study was performed in a buffer composed of 10 mM HEPES pH 7.4, 150 mM NaCl, 3 mM EDTA, 0.05% v/v Surfactant P20 (HBS-EP running buffer). Different concentrations of bispecific antibodies prepared in HBS-EP (ranging from 3.3 nM to 90 nM, 3-fold serial dilutions) were injected over the hCD28.mFc captured surface at a flow rate of 30 μL/minute. Association was monitored for 5 minutes, and the dissociation of the bispecific antibodies was monitored for 10 minutes. Binding kinetics experiments were performed at 25 or 37° C.

The specific SPR-Biacore sensorgrams were obtained by a double referencing procedure. The double referencing was performed by first subtracting the signal of each injection over a reference surface (anti-mFc) from the signal over the experimental surface (anti-mFc-captured hCD28.mFc) thereby removing contributions from refractive index changes. In addition, running buffer injections were performed to allow subtraction of the signal changes resulting from the dissociation of captured hCD28.mFc from the coupled anti-mFc surface. Kinetic association (kₐ) and dissociation (K_{d}) rate constants were determined by fitting the real-time sensorgrams to a 1:1 binding model using Scrubber v2.0c curve fitting software. Binding dissociation equilibrium constants (K_{D}) and dissociative half-lives (t1/2) were calculated from the kinetic rate constants as:

$${K}_{D} ( M ) = \frac{kd}{ka} , and t 1 / 2 ( min ) = \frac{ln ( 2 )}{60 * kd}$$

Kinetics results at 25° C. and 37° C. are presented in Tables 5 and 6, respectively, below.

TABLE 5
Kinetic and Equilibrium Binding Parameters of Anti-GPCR5D × CD28 Bispecific
Antibodies to Surface-captured hCD28.mFc at 25° C.

| | hCD28.mFc | mAb Bound | | | | t½ |
|---|---|---|---|---|---|---|
| REGN # | Capture (RU) | at 90 nM (RU) | ka (1/Ms) | kd (1/s) | KD (M) | (min) |
| bsAb17372 | 104.7 ± 0.1 | 20.1 | 3.86E+04 | 2.48E−03 | 6.44E−08 | 4.7 |
| bsAb17374 | 104.7 ± 0.3 | 30.7 | 5.86E+04 | 2.29E−03 | 3.91E−08 | 5.0 |
| bsAb17375 | 104.5 ± 0.1 | 28.8 | 4.76E+04 | 2.40E−03 | 5.05E−08 | 4.8 |
| Comparator | 105.2 ± 0.0 | −2.4 | NB* | NB* | NB* | NB* |
| 1 | | | | | | |
| Comparator | 105.0 ± 0.0 | −3.7 | NB* | NB* | NB* | NB* |
| 2 | | | | | | |
| Isotype | 104.5 ± 0.2 | 0.2 | NB* | NB* | NB* | NB* |
| Control | | | | | | |

*NB = Non-Binding

TABLE 6
Kinetic and Equilibrium Binding Parameters of Anti-GPCR5D × CD28 Bispecific
Antibodies to Surface-captured hCD28.mFc at 37° C.

| | hCD28.mFc | mAb Bound | | | | t½ |
|---|---|---|---|---|---|---|
| REGN # | Capture (RU) | at 90 nM (RU) | ka (1/Ms) | kd (1/s) | KD (M) | (min) |
| bsAb17372 | 116.7 ± 0.3 | 16.6 | 3.15E+04 | 6.32E−03 | 2.01E−07 | 1.8 |
| bsAb17374 | 116.9 ± 1.1 | 21.2 | 8.66E+04 | 8.32E−03 | 9.61E−08 | 1.4 |
| bsAb17375 | 115.3 ± 0.3 | 21.0 | 7.14E+04 | 6.58E−03 | 9.22E−08 | 1.8 |
| Comparator | 120.0 ± 0.2 | −2.6 | NB* | NB* | NB* | NB* |
| Comparator | 118.6 ± 1.0 | NA** | NA** | NA** | NA** | NA** |
| 2 | | | | | | |
| Isotype | 114.9 ± 0.6 | 0.9 | NB* | NB* | NB* | NB* |
| Control | | | | | | |

*NB = Non-Binding
**NA = Not-Available due to technical issue

### Example 3: Flow Cytometry Binding of Bispecific Anti-GPRC5D x CD28 Antibodies to Human and Cynomolgus GPRC5D and Human CD28 Expressing Cells

Characterization of hGPRC5D x CD28 bispecific antibody binding to cells using flow cytometry was determined. Binding of the GPCRC5D arm was tested using Ramos.2G6.4C10/hGPRC5D cells, which were engineered to express hGPRC5D, as well as Ramos.2G6.4C10 mfGPRC5D cells which were engineered to express mfGPRC5D. Binding of the CD28 arm was assessed using Jurkat cells which endogenously express CD28. Additionally, OPM-2 and MOLP-8 cells were also tested in binding experiments, both cell lines express endogenous GPRC5D.

The ability of antibodies to bind cells was assessed using flow cytometry. In order to assess specificity of antibody binding to GPRC5D and CD28, Ramos.2G6.4C10/hGPRC5D (engineered to express hGPRC5D), Ramos.2G6.4C10/mfGPRC5D (engineered to express mfGPRC5D), Ramos.2G6.4C10 (does not express GPRC5D), Jurkat (endogenously express CD28), MOLP-8 (endogenously express GPRC5D) and OPM-2 (endogenously express GPRC5D and CD28) cell lines were used for binding evaluation. Binding was detected by using a labeled secondary antibody and measuring fluorescence on a flow cytometer.

Ramos.2G6.4C10, Ramos.2G6.4C10/hGPRC5D, Ramos.2G6.4C10/mfGPRC5D, Jurkat, OPM-2, and MOLP-8 cells were washed and resuspended in stain buffer (1% FBS in PBS). The washed cells were filtered through a 0.4 uM cell strainer and 4×10\hat{ }4 cells/well added to the wells of a 384-well, round-bottom plate. An 8-point 1:5 dose titration of antibodies ranging from 100 nM to 6.4 pM were added to cells, with the final point of the titration containing no antibody. Cells and antibodies were incubated for 30 min at 4° C. and then washed in stain buffer. Cells were resuspended in viability dye (reconstituted in DMSO according to the manufacturer's protocol and diluted 1:1000 in PBS) alone or in viability dye containing 2 μg/ml allophycocyanin (APC) conjugated goat-anti human secondary antibody. The mixture was incubated for 30 min at 4° C. and then washed in stain buffer. Cells were resuspended in PFA (2% diluted in stain buffer) for 30 min at 4° C. After washing, cells were resuspended in stain buffer and analyzed by flow cytometry. EC₅₀ values of the antibodies were determined from a 4-parameter logistic equation over an 8-point dose response curve (including secondary only control) using GraphPad Prism software. In Prism, the 0 nM concentration was plotted as 1.3 pM.

Binding to Engineered Ramos Cell Lines (Ramos.2G6.4C10/hGPRC5D, Ramos.2G6.4C10/mfGPRC5D, Ramos.2G6.4C10): Dose dependent binding of the GPRC5DxCD28 antibodies (bsAb17372, bsAb17374 and bsAb17375) was observed in the presence of Ramos.2G6.4C10 cells engineered to express either human or Macaca fascicularis GPRC5D, whereas no binding was observed to Ramos.2G6.4C10 cells (lacking GPRC5D expression). Likewise, corresponding GRPC5D parental bivalent antibodies (mAb77036, mAb77056 and mAb77083) bound to both Ramos.2G6.4C10/hGPRC5D and Ramos.2G6.4C10/mfGPRC5D cell lines, however not Ramos.2G6.4C10. mAb77083 displayed the most potent binding. In contrast, binding of the GPRC5D bivalent comparator antibodies Comparator 1 and Comparator 2, was observed on Ramos.2G6.4C10/hGPRC5D cells but not on Ramos.2G6.4C10/mfGPRC5D cells. No binding of the isotype control antibody was observed to any of the cell lines.

Binding to OPM-2 and MOLP-8 Cell Lines (Endogenous GPRC5D): Dose dependent binding of the GPRC5DxCD28 antibodies (bsAb17372, bsAb17374 and bsAb17375) was observed in the presence of either OPM-2 or MOLP-8 cells, which endogenously express GPRC5D. These cells also endogenously express CD28, with MOLP-8 have about 10-fold the RNA levels for CD28 as OPM-2. We also observed binding of the GPRC5D bivalent parental antibodies to both cell lines, with mAb77056 only showing binding at the highest concentrations. The bivalent comparator antibodies Comparator 1 and Comparator 2, bound both OPM-2 and MOLP-8 cells, though greater max binding was observed with Comparator 2. The isotype antibody bound slightly, only at the highest concentrations.

Jurkat: Dose dependent binding of the GPRC5DxCD28 antibodies (bsAb17372, bsAb17374 and bsAb17375) was observed in the presence of Jurkat cells, which endogenously express CD28. Highest max binding was observed for bsAb17375. No binding was observed for the bivalent anti-GPRC5D antibodies (mAb77036, mAb77056 and mAb77083) nor for the anti-GPRC5D comparator antibodies (Comparator 1 and Comparator 2). Nor was binding observed for the isotype control antibody.

TABLE 7
Maximum binding and EC50 values of binding on Ramos Cells

| | Ramos/hGPRC5D | | | Ramos/mfGPRC5D | | | Ramos | | |
|---|---|---|---|---|---|---|---|---|---|
| | Max | Fold | EC50 | Max | Fold | EC50 | Max | Fold | EC50 |
| Antibody | [gMFI] | Change | [M] | [gMFI] | Change | [M] | [gMFI] | Change | [M] |
| bsAb17372 | 1.5E+06 | 698.7 | 6.79E−09 | 1.5E+06 | 772.5 | 3.364E−08 | 4.6E−08 | 2.6 | ND |
| mAb77036 | 3.1E+06 | 1016.0 | NC | 3.4E+06 | 1571.8 | 1.016E−09 | 3.8E+03 | 2.1 | ND |
| bsAb17374 | 8.4E+05 | 387.0 | 1.95E−08 | 6.8E+05 | 343.8 | NC | 4.3E+03 | 2.5 | ND |
| mAb77056 | 1.7E+06 | 557.6 | 4.39E−09 | 1.9E+06 | 874.1 | 6.709E−10 | 3.6E+03 | 1.9 | ND |
| bsAb17375 | 2.3E+06 | 1058.9 | 1.31E−08 | 2.5E+06 | 1266.5 | NC | 1.0E+04 | 5.8 | ND |
| mAb77083 | 3.4E+06 | 1126.0 | NC | 3.7E+06 | 1709.2 | 3.718E−10 | 3.6E+03 | 2.0 | ND |
| Comparator | 1.5E+06 | 707.7 | 8.09E−10 | 1.5E+04 | 7.8 | ND | 3.6E+03 | 2.1 | ND |
| 1 | | | | | | | | | |
| Comparator | 2.6E+06 | 1215.1 | 1.32E−09 | 1.3E+04 | 6.8 | ND | 3.5E+03 | 2.0 | ND |
| 2 | | | | | | | | | |
| Isotype | 1.5E+04 | 7.0 | ND | 2.4E+04 | 12.0 | ND | 1.6E+04 | 9.1 | ND |
| Control | | | | | | | | | |

TABLE 8
Maximum binding and EC50 values of binding on Endogenous Cells

| | MOLP8 | | | OPM2 | | |
|---|---|---|---|---|---|---|
| Antibody | Max [gMFI] | Fold Change | EC50 [M] | Max [gMFI] | Fold Change | EC50 [M] |
| bsAb17372 | 1.9E+05 | 33.8 | 2.01E−09 | 1.6E+05 | 26.4 | 2.132E−09 |
| mAb77036 | 1.3E+05 | 30.1 | NC | 1.0E+05 | 22.7 | NC |
| bsAb17374 | 9.3E+04 | 16.9 | NC | 7.4E+04 | 12.5 | NC |
| mAb77056 | 3.2E+04 | 7.3 | NC | 2.3E+04 | 4.9 | NC |
| bsAb17375 | 1.8E+05 | 33.5 | NC | 1.2E+05 | 20.5 | NC |
| mab77083 | 1.7E+05 | 38.0 | NC | 1.0E+05 | 22.0 | NC |
| Comparator 1 | 3.1E+04 | 5.6 | ND | 1.4E+04 | 2.4 | NC |
| Comparator 2 | 1.7E+05 | 30.8 | ND | 1.7E+05 | 28.2 | NC |
| Isotype | 1.1E+04 | 1.9 | ND | 1.5E+04 | 2.5 | ND |
| Control | | | | | | |

TABLE 9
Maximum binding and EC50 values of binding on Jurkat

Jurkat

| Antibody | Max [gMFI] | Fold Change | EC50 [M] |
|---|---|---|---|
| bsAb17372 | 1.2E+05 | 65.9 | NC |
| mAb77036 | 1.9E+03 | 1.1 | ND |
| bsAb17374 | 1.2E+05 | 66.3 | NC |
| mAb77056 | 1.8E+03 | 1.1 | ND |
| bsAb17375 | 1.4E+05 | 75.7 | NC |
| mAb77083 | 2.0E+03 | 1.2 | ND |
| Comparator 1 | 2.1E+03 | 1.1 | ND |
| Comparator 2 | 2.6E+03 | 1.4 | ND |
| Isotype Control | 2.3E+03 | 1.2 | ND |

Tables 7, 8 and 9-Geometric Mean Fluorescence Intensity (gMFI) values were plotted using GraphPad Prism and EC₅₀ values of the antibodies were determined using a four-parameter, variable slope, non-linear regression equation over an 8-point dose-response curve, with a 1:5 titration of antibody ranging from 6.4 pM to 100 nM, and a no antibody condition. Maximum gMFI, is the highest gMFI along the dose response curve and the fold binding is the maximum gMFI divided by the gMFI value from secondary antibody alone.
Abbreviations: ND: Not Determined because no dose dependent response was observed; NC: Not calculated because the data did not fit a 4-parameter logistic equation

### Example 4: Characterization of Bispecific Anti-GPRC5D x CD28 Antibody Activity in an Engineered Reporter Assay

Two signals, “signal 1” & “signal 2”, are required for proper T cell activation. “Signal 1” is induced by binding of the T cell receptor (TCR) on T cells to peptide-bound major histocompatibility complex (MHC) molecules on antigen presenting cells (APCs). “Signal 2” is provided by engaging co-stimulatory receptors on T-cells, such as CD28, with their ligands, such as cluster of differentiation 80 or 86 (CD80/CD86), on APCs. Therefore, activation of CD28 signaling provides a targeted approach to enhance existing TCR signaling. GPRC5D x CD28 bispecific antibodies are designed to mimic the natural ligands of CD28, by bridging GPRC5D⁺ target cells with CD28⁺ T cells, to provide “signal 2” in order to enhance the activation of T cells in the presence of a “signal 1” provided by a tumor-associated antigen (TAA) x CD3.

The ability of GPRC5D x CD28 bispecific antibodies to activate human primary T-cells by engaging GPRC5D on target cells and CD28 on T-cells, to deliver “signal 2”, was evaluated in an engineered reporter assay. In this assay, Jurkat cells (endogenously express CD28) are engineered to express the reporter gene luciferase under the control of the transcription factor NF-kB (NFkB-Luc). Target cells are Ramos.2G6.4C10 cells engineered to express human GPRC5D (hGPRC5D) or Macaca fascicularis GPRC5D (mfGPRC5D). Other target cells include OPM-2 and MOLP-8 which endogenously express hGPRC5D. In Jurkat reporter assays with target cells (Ramos.2G6.4C10/hGPRC5D, Ramos.2G6.4C10/mfGPRC5D or Ramos.2G6.4C10), aCD20 x CD3 bispecific antibody is used to provide “signal 1”, while assays using target cells OPM-2 or MOLP-8 utilized BCMA x CD3 to provide “signal 1.” The ability of GPRC5D x CD28 antibodies to specifically engage GPRC5D on target cells and facilitate CD28 clustering/activation of NFκB mediated luciferase production was evaluated.

One day before the experiment, Jurkat reporter cells were split to 7.5×10⁵ cells/mL in growth media (RPMI+10% FBS+Penicillin/Streptomycin/L-glutamine (P/S/G)+1 μg/ml Puromycin). On the day of the experiment Jurkat/NFkB-luc cells were resuspended in assay media (RPMI supplemented with 10% FBS+P/S/G) and were added to 384-well white plates at a final concentration of 1.5×10⁴ cells/well. Target cells, resuspended in assay media, were then added to wells at a concentration of either 4×10³ cells/well (Ramos.2G6.4C10/hGPRC5D, Ramos.2G6.4C10/mfGPRC5D, and Ramos.2G6.4C10 cells) or 6.5×10³ cells/well (OPM-2 and MOLP-8 cells). A fixed amount of either CD20 x CD3 (0.1 nM) or BCMA x CD3 (0.5 nM) was prepared in assay media and added to the appropriate wells. Subsequently, GPRC5D x CD28 and isotype control antibodies were prepared in assay media and titrated from 100 nM to 95.4 fM in a 1:5 dilution, the final point of the 12-point dilution containing no titrated antibody and added, in duplicate, to the appropriate wells. Plates were incubated at 37° C. and 5% CO2 for 5 hours and then ONE-Glo luciferase substrate was added to each well according to manufacturer's instructions. The luciferase activity was recorded as a luminescence signal using the ENVISION plate reader and expressed as relative light units (RLU). The EC₅₀ values were determined by a 4-parameter logistic equation over a 12-point response curve using GraphPad Prism™. Signal recorded for the 12th point on the dilution curve (no titrated antibody) was plotted at 24 fM. Maximal RLU is given as the mean max response detected within the tested dose range.

In the presence of Jurkat/NFkB-Luc cells and target cells expressing CD20 or BCMA, the addition of primary stimulation (CD20xCD3 or BCMAxCD3, respectively) lead to increased baseline activity compared to conditions lacking primary stimulation. Additionally, in the absence of primary stimulation, GPRC5D x CD28 antibodies (bsAb17372, bsAb17374, bsAb17375) led to a minor, dose dependent increase in NF-kB activity, which was greatly increased in the presence of ‘signal 1’ (either CD20xCD3 or BCMAxCD3 depending on the target cell line).

In the presence of Ramos.2G6.4C10/hGPRC5D target cells and CD20xCD3 primary stimulation, the GPRC5D x CD28 molecules (bsAb17372, bsAb17374, bsAb17375) led to a dose dependent increase in luciferase activity, while the isotype control antibody did not result in an increase. The EC₅₀ was lowest for bsAb17372. In the absence of CD20 x CD3 primary stimulation the GPRC5D x CD28 molecules led to a slight increase in activity at the highest tested antibody concentrations with less potency than seen in the presence of CD20 x CD3.

In the presence of Ramos.2G6.4C10/mfGPRC5D target cells and CD20 x CD3 primary stimulation, the GPRC5D x CD28 molecules (bsAb17372, bsAb17374, bsAb17375) led to a slight increase in activity at the highest tested antibody concentrations, with no potency value assigned, due to lack of signal plateau, while the isotype control antibody did not result in an increase. The EC₅₀ was lowest for bsAb17372. In the absence of CD20 x CD3 primary stimulation the GPRC5D x CD28 molecules led to a slight increase in activity at the highest tested antibody concentrations with less potency than seen in the presence of CD20 x CD3.

In the presence of Ramos.2G6.4C10 target cells lacking GPRC5D stimulation, the GPRC5D x CD28 molecules (bsAb17372, bsAb17374, bsAb17375) led to a dose dependent increase in luciferase activity, while the isotype control antibody did not result in an increase. In the absence of CD20 x CD3 primary stimulation the GPRC5D x CD28 molecules led to a minimal increase in activity (˜10-fold lower than in the presence of CD20 xCD3) only at the highest tested antibody concentrations with less potency than seen in the presence of CD20 x CD3.

In the presence of OPM-2 target cells and BCMA x CD3 primary stimulation, the GPRC5D x CD28 molecules (bsAb17372 and bsAb17375) led to a dose dependent increase in luciferase activity, while bsAb17374 led to an increase in activity at the highest points tested. Out of the GPRC5D x CD28 molecules, bsAb17372 was the most potent. The isotype control antibody did not result in an increase in reporter activity. In the absence of BCMA x CD3 primary stimulation the GPRC5D x CD28 molecules did not lead to an increase in NF-kB activity.

In the presence of MOLP-8 target cells and BCMA x CD3 primary stimulation, the GPRC5D x CD28 molecules (bsAb17372 and bsAb17375) led to a dose dependent increase in luciferase activity, while bsAb17374 led to an increase in activity at the highest points tested. Out of the GPRC5D x CD28 molecules, bsAb17372 was the most potent. The isotype control antibody did not result in an increase in reporter activity. In the absence of BCMA x CD3 primary stimulation the GPRC5D x CD28 molecules led to a slight increase in activity at the highest tested antibody concentrations with no potency value assigned, due to lack of signal plateau.

TABLE 10
Potency values, EC₅₀ [M], for GPRC5D × CD28
antibodies in the presence of CD3 Stimulation

| | Ramos | Ramos | | | | Jurkat/ |
|---|---|---|---|---|---|---|
| Antibody | hGPRC5D | mfGPRC5D | Ramos | MOLP-8 | OPM-2 | NFkB-Luc |
| bsAb17372 | 3.00E−12 | 3.33E−11 | NC | 2.16E−10 | 3.96E−10 | ND |
| bsAb17374 | 4.55E−10 | 1.31E−09 | NC | NC | NC | ND |
| bsAb17375 | 1.12E−11 | 9.38E−11 | NC | 1.22E−09 | 1.93E−09 | ND |
| Isotype | ND | ND | ND | ND | ND | ND |
| Control | | | | | | |

TABLE 11
Maximum Reporter Activity, Max RLU, for GPRC5D ×
CD28 antibodies in the presence of CD3 stimulation

| | Ramos | Ramos | | | | Jurkat/ |
|---|---|---|---|---|---|---|
| Antibody | hGPRC5D | mfGPRC5D | Ramos | MOLP-8 | OPM-2 | NFkB-Luc |
| bsAb17372 | 6.53E+05 | 5.68E+05 | 7.81E+04 | 2.69E+05 | 1.63E+05 | 1.69E+04 |
| bsAb17374 | 6.16E+05 | 5.19E+05 | 8.04E+04 | 2.20E+05 | 1.25E+05 | 9.40E+03 |
| bsAb17375 | 6.98E+05 | 6.05E+05 | 7.53E+04 | 2.24E+05 | 1.14E+05 | 8.36E+03 |
| Isotype | 6.15E+04 | 3.37E+04 | 3.10E+04 | 5.73E+04 | 4.52E+04 | 6.32E+03 |
| Control | | | | | | |

TABLE 12
Potency values, EC₅₀ [M], for GPRC5D × CD28
antibodies in the absence of CD3 stimulation

| | Ramos | Ramos | | | | Jurkat/ |
|---|---|---|---|---|---|---|
| Antibody | hGPRC5D | mfGPRC5D | Ramos | MOLP-8 | OPM-2 | NFkB-Luc |
| bsAb17372 | 6.17E−11 | 6.26E−10 | NC | NC | 8.97E−10 | ND |
| bsAb17374 | NC | NC | NC | NC | NC | ND |
| bsAb17375 | NC | NC | NC | NC | 1.12E−08 | ND |
| Isotype | ND | ND | ND | ND | ND | ND |
| Control | | | | | | |

TABLE 13
Maximum Reporter Activity, Max RLU, for GPRC5D ×
CD28 antibodies in the absence of CD3 stimulation

| | Ramos | Ramos | | | | Jurkat/ |
|---|---|---|---|---|---|---|
| Antibody | hGPRC5D | mfGPRC5D | Ramos | MOLP-8 | OPM-2 | NFkB-Luc |
| bsAb17372 | 1.12E+05 | 1.40E+05 | 7.62E+03 | 1.12E+05 | 1.13E+04 | 1.55E+04 |
| bsAb17374 | 2.70E+05 | 2.45E+05 | 1.03E+04 | 2.70E+05 | 1.06E+04 | 1.08E+04 |
| bsAb17375 | 2.01E+05 | 2.41E+05 | 9.16E+03 | 2.01E+05 | 1.12E+04 | 8.40E+03 |
| isotype | 7.12E+03 | 6.72E+03 | 6.92E+03 | 8.82E+03 | 6.92E+03 | 6.06E+03 |
| Control | | | | | | |

Tables 10-13: Abbreviations: ND: Not Determined because no dose dependent response was observed
NC: Not calculated because the data did not fit a 4-parameter logistic equation
Max (RLU) is the highest mean RLU value within tested dose-range.
†While all data points are used to determine Maximum luciferase activity, for EC50 calculation the values for the highest 3 antibody concentrations were removed due to a “hook effect”.

### Example 5: Characterization of T-Cell Activation Activity of Bispecific Anti-GPRC5D x CD28 Antibodies

Two signals, “signal 1” & “signal 2”, are required for proper T cell activation. “Signal 1” is induced by binding of the T cell receptor (TCR) on T cells to peptide-bound major histocompatibility complex (MHC) molecules on antigen presenting cells (APCs). “Signal 2” is provided by engaging co-stimulatory receptors, such as CD28, on T cells with ligands, such as cluster of differentiation 80 or 86 (CD80/CD86), present on APCs. Therefore, activation of CD28 signaling provides a targeted approach to enhance existing TCR signaling. GPRC5D x CD28 bispecific antibodies are designed to mimic the natural ligands of CD28, by bridging GPRC5D⁺ target cells with CD28⁺ T cells, to provide “signal 2” in order to enhance the activation of T cells in the presence of a “signal 1” provided by an allogeneic response provided by the APC.

The ability of GPRC5D x CD28 bispecific antibodies to activate human primary T-cells by engaging GPRC5D and CD28 to deliver “signal 2”, as determined by IL2 release and IFN-γ release, was evaluated in the presence of a myeloma cell line, OPM-2, using an allogenic response provided by the cell line to serve as “signal 1.”

Human peripheral blood mononuclear cells (PBMCs) were isolated from a healthy donor leukocyte pack from Precision for Medicine (Donor 555254) using the EasySep™ Direct Human PBMC Isolation Kit, following the manufacturers recommended protocol and frozen down. CD3⁺ T-cells were isolated from thawed PBMC's using an EasySep™ Human CD3⁺ T Cell Isolation Kit from StemCell Technologies and following the manufacturer's recommended instructions.

Enriched CD3⁺ T-cells, resuspended in stimulation media, were added into 96-well round bottom plates at a concentration of 1×10⁵ cells/well. OPM-2 cells were added to CD3⁺ T-cells at a final concentration of 2×10⁴ cells/well. Subsequently, test antibodies were titrated from 298 fM to 500 nM in a 1:6 dilution and added to wells. The final point of the 10-point dilution contained no titrated antibody. Plates were incubated for 96 hours at 37° C., 5% CO2 and 20 μL supernatant was removed and used for measuring IL2 as well as 20 μl of supernatant for measuring IFNγ. The amount of cytokine in assay supernatant was determined using AlphaLisa kits from PerkinElmer following the manufacturer's protocol with modifications recommended by the manufacturer for enhanced cytokine detection. These modifications included adjusting the supernatant volume for testing from 5 μl to 20 μl, while maintaining the recommended bead and antibody concentration, and increasing the incubation time with the anti-analyte acceptor bead/biotinylated antibody mix from one hour to two hours. The cytokine measurements were acquired on Perkin Elmer's multilabel plate reader Envision and values were reported as μg/mL. All serial dilutions were tested in duplicate. The EC₅₀ values of the antibodies were determined from a four-parameter logistic equation over a 10-point dose-response curve using GraphPad Prism™ software. Maximal IL2 & IFNγ is given as the mean max response detected within the tested dose range.

In the presence of T-cells and an allogeneic cell line expressing GPRC5D, OPM-2, increased IL2 & IFNγ release was observed in the presence of GPRC5DxCD28 antibodies.

In the presence of OPM-2 target cells and allogeneic stimulation, the GPRC5D x CD28 molecules (bsAb17372, bsAb17374 and bsAb17375) led to a dose dependent increase in IL2 release. bsAb17372 led to the highest maximum cytokine release among all molecules tested. An increase in IL-2 was observed for Control 1, however only at highest tested concentrations and EC₅₀ values could not be generated, as the signal did not plateau.

In the presence of OPM-2 target cells and allogeneic stimulation, the GPRC5D x CD28 molecules (bsAb17372, bsAb17374 and bsAb17375) led to a dose dependent increase in IFNγ release. bsAb17372 led to the highest maximum cytokine release among all molecules tested. An increase in IFNγ was observed for Control 1, however only at highest tested concentrations and EC₅₀ values could not be generated, as the signal did not plateau.

TABLE 14
Maximum IL2 & IFNγ release and potency values of Antibodies

| | OPM-2 | | | |
|---|---|---|---|---|
| | IL2 | | IFNγ | |
| | Max | EC50 | Max | EC50 |
| Antibodies | [pg/ml] | [M] | [pg/ml] | [M] |
| bsAb17372 | ^{†}2.77E+04 | NC | 9.60E+03 | 2.121E−08 |
| bsAb17374 | ^{†}2.02E+04 | NC | 4.64E+03 | 3.648E−08 |
| bsAb17375 | ^{†}1.57E+04 | 2.594E−09^{†} | 3.45E+03 | 1.766E−08 |
| Control 1 | 1.03E+04 | NC | 4.19E+03 | NC |

Abbreviations:
ND: Not Determined because a dose dependent response was not observed
NC: Not calculated because the data did not fit a 4-parameter logistic equation
Maximum (pg/ml) is the highest mean pg/ml value within tested dose-range.
^{†}While all data points are used to determine Max [pg/ml] of cytokine released, for EC50 calculation the values for the highest antibody concentrations were removed due to a “hook effect”

### Example 6: FACS Based Cytotoxicity Assay of Bispecific Anti-GPRC5D x CD28 Antibodies

The anti-GPRC5D x CD28 bispecific antibodies bsAb17372, bsAb17374, bsAb17375 were tested for their ability to enhance multiple myeloma (MM) MOLP-8 and OPM-2 cell killing and human T cell activation mediated by anti-BCMA x CD3 antibody REGN5458 (U.S. Pat. No. 11,384,153). GPRC5D x CD28 enhancement of BCMA x CD3 targeted killing was evaluated in a 96-hour cytotoxicity assay targeting multiple myeloma (MM) cells MOLP8 and OPM-2. Briefly, human PBMCs were plated in supplemented RPMI media at 1×10⁶ cells/mL and incubated overnight at 37° C. in order to enrich for lymphocytes by depleting adherent macrophages, dendritic cells, and some monocytes. The following day, MM cells were labeled with 1 uM of the fluorescent tracking dye CFDA-SE and the adherent cell-depleted naïve PBMC were labeled with 1 uM of the fluorescent tracking dye CellTrace Violet. Labeled target cells and PBMC (Effector/Target cell 5:1 ratio) were co-incubated with a serial dilution of BCMAxCD3 bispecific antibody REGN5458 (concentration range: 67 nM to 0.7 pM) and a fixed concentration of GPRC5DxCD28 costimulatory molecules bsAb17372, bsAb17374, bsAb17375, or Control 1 at 16.7 nM for 96 hours at 37° C. Alternatively, labeled target cells and PBMC (Effector/Target cell 5:1 ratio) were co-incubated with a fixed concentration of BCMA x CD3 bispecific antibody REGN5458 at 40 pM and a serial dilution of GPRC5DxCD28 costimulatory molecules bsAb17372, bsAb17375, or Control 1 (concentration range: 20 nM to 3 pM) for 96 hours at 37° C.

Cells were harvested from the plates and analyzed by FACS on a FACS BD LSRFortessa-X20. For FACS analysis, cells were stained with a Fixable Live/Dead Far Red reactive (Invitrogen) dye. 20,000 counting beads were added to each well immediately before FACS analysis and 10,000 beads were collected for each sample. For the assessment of specificity of killing, cells were gated on live CFDA-SE labeled populations. Percent of live population was recorded and used for the calculation of survival.

T cell activation was assessed by incubating cells with directly conjugated antibodies to CD2, CD4, CD8, and CD25. The percentage of CD8+ cells and CD4+ T cells expressing CD25 was reported as the measure of T cell activation. Additionally, as T cells proliferate, CellTrace Violet is diluted. The percentage of CD8+ cells and CD4+ T cells with diluted CellTraceViolet was reported as the measure for T cell proliferation. EC50 values were calculated using 4-parameter non-linear regression analysis in Prism software.

In an assay where a constant concentration of GPRC5D x CD28 or Control 1 was added to a titration of BCMA x CD3 or Control 2, it was found that bsAb17372 and bsAb17375 enhanced the potency of BCMA x CD3 mediated MM cytotoxicity and/or T cell activation. No enhancement of BCMA x CD3 activity was observed with bsAb17374 or Control 1 (Table 15). GPRC5D x CD28 or Control 1 did not show any activity in the presence of Control 2.

In an assay where a titration of GPRC5D x CD28 or Control 1 was added to constant concentration of BCMA x CD3, bsAb17372 and bsAb17375 enhanced the cytotoxicity and T cell activation mediated by 40 pM BCMA x CD3 in dose-dependent manner for GPRC5D positive target cells MOLP-8 and OPM-8. Non-targeting CD28 control antibody showed activity only at 12.5 nM or above (Table 16).

In summary, co-stimulation increased the potency of targeted cytotoxicity and T cell activation when compared to what was observed with BCMAxCD3 alone.

TABLE 15
Fold Change in BCMA × CD3 EC50 Potency
for target cytotoxicity and T cell activation

| | Cell Kill | | CD8 T cell activation | |
|---|---|---|---|---|
| | EC50 [M] | | (CD25+) EC50 [M] | |
| | OPM- | MOLP- | OPM- | MOLP- |
| Ab | 2 | 8 | 2 | 8 |
| bsAb17372 + BCMA × CD3 | 3.6 | 3.3 | 5.7 | 6.9 |
| bsAb17374 + BCMA × CD3 | 2.0 | 0.9 | 1.1 | 1.2 |
| bsAb17375 + BCMA × CD3 | 2.1 | 3.4 | 3.1 | 3.3 |
| Control 1 + BCMA × CD3 | 2.0 | NC | 1.9 | 0.8 |

NC: Not calculated because the data did not fit a 4-parameter logistic equation
EC50 for BCMA × CD3 alone divided by EC50 for BCMA × CD3 in the presence of 16.7 nM GPRC5D × CD28 costim

TABLE 16
EC50 values for target cytotoxicity and T cell activation mediated by
GPRC5D × CD28 costim in the presence of 40 pM BCMA × CD3

| | OPM-2 | | | MOLP-8 | | |
|---|---|---|---|---|---|---|
| | | T cell activation (CD25+) | | | T cell activation (CD25+) | |
| | Cell Kill | EC50 [M] | | Cell Kill | EC50 [M] | |
| Antibody | EC50 [M] | CD8 T cells | CD4 T cells | EC50 [M] | CD8 T cells | CD4 T cells |
| bsAb17372 | 5.81E−09 | 1.87E−09 | 1.37E−09 | 7.81E−09 | 4.75E−09 | 3.52E−09 |
| bsAb17375 | 3.39E−09 | 1.22E−09 | 9.39E−10 | 9.05E−09 | 5.22E−09 | 4.85E−09 |
| Control 1 | NC | NC | NC | NC | NC | NC |

NC: Not calculated because the data did not fit a 4-parameter logistic equation.

### Example 7: In Vivo Antitumor-Efficacy of Bispecific Anti-GPRC5D x CD28 Antibodies in a Xenogenic Tumor Study (H929 Cells)

To determine the in vivo anti-tumor efficacy of GPRC5D x CD28 bispecific antibodies in combination with a BCMA x CD3 bispecific antibody, a xenogenic tumor study was performed. On Day 0, immunodeficient NOD.Cg-Prkdc^{scid}∥2rg^{tm1Wjl}/SzJ (NSG) mice were subcutaneously injected with 10×10⁶ H929 tumor cells plus 0.5×10⁶ human peripheral blood mononuclear cells (PBMC) from a normal, healthy donor. On Day 12, the mice (n=5 per group) were administered either Control 2 or a BCMA x CD3 (REGN5458) bispecific antibody at 0.05 mg/kg, in combination with Control 1, or a GPRC5D x CD28 bispecific antibody (bsAb17372) at 4 mg/kg, 0.4 mg/kg or 0.04 mg/kg. The mice were administered these antibodies twice more on days 19 and 26, for a total of three doses. Tumor growth was assessed over 28 days twice weekly by using calipers and calculated by the following formula: (length×width²)/2. As a positive control, a group of mice (n=5) was given only H929 cells and PBMCs, but not antibody (PBS-treated group). These studies demonstrate that while monotherapy with BCMA x CD3 bispecific antibody shows very modest anti-tumor efficacy, combination treatment with BCMA x CD3 bispecific antibody (REGN5458) plus GPRC5D x CD28 (bsAb17372) results in more potent, combinatorial anti-tumor efficacy that is superior to BCMA x CD3 bispecific antibody (REGN5458) plus Control 1.

On Day 0, immunodeficient NOD.Cg-Prkdc^{scid}∥2rg^{tm1Wjl}/SzJ (NSG) mice were subcutaneously injected with 10×10⁶ H929 tumor cells plus 0.5×10⁶ human peripheral blood mononuclear cells (PBMC) from a normal, healthy donor. On Day 12, the mice (n=5 per group) were administered either Control 2 or a BCMA x CD3 (REGN5458) bispecific antibody at 0.05 mg/kg, in combination with Control 1, or a GPRC5D x CD28 bispecific antibody (bsAb17372) at 4 mg/kg, 0.4 mg/kg or 0.04 mg/kg. The mice were administered these antibodies twice more on days 19 and 26, for a total of three doses. Tumor growth was assessed over 28 days twice weekly by using calipers and calculated by the following formula: (length×width²)/2. As a positive control, a group of mice (n=5) was given only H929 cells and PBMCs, but not antibody (PBS-treated group).

BCMA x CD3 bispecific antibody (REGN5458) plus Control 1 provides very modest anti-tumor efficacy compared to Control 2 plus Control 1. The combination of BCMA x CD3 bispecific antibody (REGN5458) plus GPRC5D x CD28 bispecific antibody (bsAb17372) results in mean tumor volumes that are lower than mice receiving BCMA x CD3 bispecific antibody (REGN5458) plus Control 1 at 4 mg/kg and 0.4 mg/kg, but the combinatorial effect is lost at 0.04 mg/kg.

These studies demonstrate that while monotherapy with BCMA x CD3 bispecific antibody shows very modest anti-tumor efficacy, combination treatment with BCMA x CD3 bispecific antibody (REGN5458) plus GPRC5D x CD28 (bsAb17372) results in more potent, combinatorial anti-tumor efficacy that is superior to BCMA x CD3 bispecific antibody (REGN5458) plus Control 1.

TABLE 17A
Tumor Volume at Day 9

| | Mean Tumor | Tumor | Number of |
|---|---|---|---|
| Antibody | Volume (mm3) | Volume SEM | mice still |
| Treatment | on Day 9 | on Day 9 | alive on day 9 |
| Control 2 (0.05 mg/kg) + Control 1 (4 mg/kg) | 106.0 | 14.6 | 5 of 5 |
| BCMA × CD3 bsAb (0.05 mg/kg) + Control 1 | 113.1 | 27.0 | 8 of 8 |
| (4 mg/kg) | | | |
| BCMA × CD3 bsAb (0.05 mg/kg) + | 95.0 | 20.7 | 5 of 5 |
| GPRC5D × CD28 bsAb17372 (4 mg/kg) | | | |
| BCMA × CD3 bsAb (0.05 mg/kg) + | 92.2 | 23.0 | 5 of 5 |
| GPRC5D × CD28 bsAb17372 (0.4 mg/kg) | | | |
| BCMA × CD3 bsAb (0.05 mg/kg) + | 114.8 | 17.5 | 5 of 5 |
| GPRC5D × CD28 bsAb17372 (0.04 mg/kg) | | | |

TABLE 17B
Tumor Volume at Day 13

| | Mean Tumor | Tumor | Number of |
|---|---|---|---|
| Antibody | Volume (mm3) | Volume SEM | mice still |
| Treatment | on Day 13 | on Day 13 | alive on day 13 |
| Control 2 (0.05 mg/kg) + Control 1 (4 mg/kg) | 225.0 | 53.3 | 5 of 5 |
| BCMA × CD3 bsAb (0.05 mg/kg) + Control 1 | 225.8 | 62.3 | 8 of 8 |
| (4 mg/kg) | | | |
| BCMA × CD3 bsAb (0.05 mg/kg) + | 153.2 | 44.2 | 5 of 5 |
| GPRC5D × CD28 bsAb17372 (4 mg/kg) | | | |
| BCMA × CD3 bsAb (0.05 mg/kg) + | 158.4 | 26.7 | 5 of 5 |
| GPRC5D × CD28 bsAb17372 (0.4 mg/kg) | | | |
| BCMA × CD3 bsAb (0.05 mg/kg) + | 241.8 | 59.1 | 5 of 5 |
| GPRC5D × CD28 bsAb17372 (0.04 mg/kg) | | | |

TABLE 17C
Tumor Volume at Day 16

| | Mean Tumor | Tumor | Number of |
|---|---|---|---|
| Antibody | Volume (mm3) | Volume SEM | mice still |
| Treatment | on Day 16 | on Day 16 | alive on day 16 |
| Control 2 (0.05 mg/kg) + Control 1 (4 mg/kg) | 469.8 | 71.6 | 5 of 5 |
| BCMA × CD3 bsAb (0.05 mg/kg) + Control 1 | 310.4 | 90.5 | 8 of 8 |
| (4 mg/kg) | | | |
| BCMA × CD3 bsAb (0.05 mg/kg) + | 157.2 | 74.0 | 5 of 5 |
| GPRC5D × CD28 bsAb17372 (4 mg/kg) | | | |
| BCMA × CD3 bsAb (0.05 mg/kg) + | 188.2 | 48.5 | 5 of 5 |
| GPRC5D × CD28 bsAb17372 (0.4 mg/kg) | | | |
| BCMA × CD3 bsAb (0.05 mg/kg) + | 401.0 | 106.0 | 5 of 5 |
| GPRC5D × CD28 bsAb17372 (0.04 mg/kg) | | | |

TABLE 17D
Tumor Volume at Day 19

| | Mean Tumor | Tumor | Number of |
|---|---|---|---|
| Antibody | Volume (mm3) | Volume SEM | mice still |
| Treatment | on Day 19 | on Day 19 | alive on day 19 |
| Control 2 (0.05 mg/kg) + Control 1 (4 mg/kg) | 687.4 | 113.0 | 5 of 5 |
| BCMA × CD3 bsAb (0.05 mg/kg) + Control 1 | 515.4 | 141.5 | 8 of 8 |
| (4 mg/kg) | | | |
| BCMA × CD3 bsAb (0.05 mg/kg) + | 218.0 | 115.2 | 5 of 5 |
| GPRC5D × CD28 bsAb17372 (4 mg/kg) | | | |
| BCMA × CD3 bsAb (0.05 mg/kg) + | 298.2 | 89.9 | 5 of 5 |
| GPRC5D × CD28 bsAb17372 (0.4 mg/kg) | | | |
| BCMA × CD3 bsAb (0.05 mg/kg) + | 738.8 | 195.3 | 5 of 5 |
| GPRC5D × CD28 bsAb17372 (0.04 mg/kg) | | | |

TABLE 17E
Tumor Volume at Day 22

| | Mean Tumor | Tumor | Number of |
|---|---|---|---|
| Antibody | Volume (mm3) | Volume SEM | mice still |
| Treatment | on Day 22 | on Day 22 | alive on day 22 |
| Control 2 (0.05 mg/kg) + Control 1 (4 mg/kg) | 1203.0 | 128.7 | 5 of 5 |
| BCMA × CD3 bsAb (0.05 mg/kg) + Control 1 | 719.6 | 210.6 | 8 of 8 |
| (4 mg/kg) | | | |
| BCMA × CD3 bsAb (0.05 mg/kg) + | 401.0 | 222.9 | 5 of 5 |
| GPRC5D × CD28 bsAb17372 (4 mg/kg) | | | |
| BCMA × CD3 bsAb (0.05 mg/kg) + | 556.4 | 160.8 | 5 of 5 |
| GPRC5D × CD28 bsAb17372 (0.4 mg/kg) | | | |
| BCMA × CD3 bsAb (0.05 mg/kg) + | 1154.0 | 346.4 | 5 of 5 |
| GPRC5D × CD28 bsAb17372 (0.04 mg/kg) | | | |

TABLE 17F
Tumor Volume at Day 26

| | Mean Tumor | Tumor | Number of |
|---|---|---|---|
| | Volume (mm3) | Volume SEM | mice still |
| Antibody Treatment | on Day 26 | on Day 26 | alive on day 26 |
| Control 2 (0.05 mg/kg) + Control 1 (4 mg/kg) | 1995.6 | 65.9 | 5 of 5 |
| BCMA × CD3 bsAb (0.05 mg/kg) + Control 1 | 1130.9 | 353.1 | 8 of 8 |
| (4 mg/kg) | | | |
| BCMA × CD3 bsAb (0.05 mg/kg) + | 708.2 | 410.1 | 5 of 5 |
| GPRC5D × CD28 bsAb17372 (4 mg/kg) | | | |
| BCMA × CD3 bsAb (0.05 mg/kg) + | 1124.8 | 306.4 | 5 of 5 |
| GPRC5D × CD28 bsAb17372 (0.4 mg/kg) | | | |
| BCMA × CD3 bsAb (0.05 mg/kg) + | 1574.3 | 478.8 | 4 of 5 |
| GPRC5D × CD28 bsAb17372 (0.04 mg/kg) | | | |

TABLE 17G
Tumor Volume at Day 28

| | Mean Tumor | Tumor | Number of |
|---|---|---|---|
| | Volume (mm3) | Volume SEM | mice still |
| Antibody Treatment | on Day 28 | on Day 28 | alive on day 28 |
| Control 2 (0.05 mg/kg) + Control 1 (4 mg/kg) | | | 0 of 5 |
| BCMA × CD3 bsAb (0.05 mg/kg) + Control 1 | 986.2 | 388.6 | 6 of 8 |
| (4 mg/kg) | | | |
| BCMA × CD3 bsAb (0.05 mg/kg) + | 1016.6 | 549.6 | 5 of 5 |
| GPRC5D × CD28 bsAb17372 (4 mg/kg) | | | |
| BCMA × CD3 bsAb (0.05 mg/kg) + | 1266.0 | 344.7 | 4 of 5 |
| GPRC5D × CD28 bsAb17372 (0.4 mg/kg) | | | |
| BCMA × CD3 bsAb (0.05 mg/kg) + | | | 0 of 5 |
| GPRC5D × CD28 bsAb17372 (0.04 mg/kg) | | | |

### Example 8: In Vivo Antitumor-Efficacy of Bispecific Anti-GPRC5D x CD28 Antibodies in a Xenogenic Tumor Study (MOLP-8 Cells)

To determine the in vivo anti-tumor efficacy of GPRC5D x CD28 bispecific antibodies (bsAb) in combination with a BCMA x CD3 bispecific antibody, a xenogenic tumor study was performed. On Day-13, immunodeficient NOD.Cg-Prkdc^{scid}∥2rg^{tm1Wjl}/SzJ (NSG) mice were intraperitoneally injected with 4×10⁶ human peripheral blood mononuclear cells (PBMC) from a normal, healthy donor. On Day 0, the mice were intravenously administered 2×10⁶ BCMA⁺GPRC5D⁺MOLP-8 human multiple myeloma tumor cells that were engineered to also express firefly luciferase (MOLP-8-luciferase cells). The mice (n=5 per group) were then immediately administered either Control 2 or a BCMA x CD3 (REGN5458) bispecific antibody at 1 mg/kg, in combination with Control 1, or a GPRC5D x CD28 bispecific antibody (either bsAb17372 or bsAb17375) at 4 mg/kg. The mice were administered these antibodies twice more on days 7 and 14, for a total of three doses. Tumor growth was assessed over 37 days by measuring tumor bioluminescence (BLI) in anesthetized animals. As a positive control, a group of mice (n=5) was given only MOLP-8-luciferase cells and PBMCs, but not antibody (PBS-treated group). In order to measure background BLI levels, a group of mice (n=5) were untreated and did not receive tumors, PBMC, or antibody (No Tumor group). These studies demonstrate that while monotherapy with BCMA x CD3 bispecific antibody (REGN5458) demonstrates modest anti-tumor efficacy, combination treatment with BCMA x CD3 bispecific antibody (REGN5458) plus GPRC5D x CD28 (bsAb17372 or bsAb17375) results in more potent, combinatorial anti-tumor efficacy that is superior to either therapy alone.

On Day-13, immunodeficient NOD.Cg-Prkdc^{scid}∥2rg^{tm1Wjl}/SzJ (NSG) mice were intraperitoneally injected with 4×10⁶ human peripheral blood mononuclear cells (PBMC) from a normal, healthy donor. On Day 0, the mice were intravenously administered 2×10⁶ BCMA⁺GPRC5D⁺MOLP-8 human multiple myeloma tumor cells that were engineered to also express firefly luciferase (MOLP-8-luciferase cells). The mice (n=5 per group) were then immediately administered either Control 2 or a BCMA x CD3 (REGN5458) bispecific antibody at 1 mg/kg, in combination with Control 1, or a GPRC5D x CD28 bispecific antibody (either bsAb17372 or bsAb17375) at 4 mg/kg. The mice were administered these antibodies twice more on days 7 and 14, for a total of three doses. Tumor growth was assessed over 37 days by measuring tumor bioluminescence (BLI) in anesthetized animals. As a positive control, a group of mice (n=5) was given only MOLP-8-luciferase cells and PBMCs, but not antibody (PBS-treated group). In order to measure background BLI levels, a group of mice (n=5) were untreated and did not receive tumors, PBMC, or antibody (No Tumor group).

BLI imaging was used to measure tumor burden. Mice were injected IP with 150 mg/kg of the luciferase substrate D-luciferin suspended in PBS. Five minutes after this injection, BLI imaging of the mice was performed under isoflurane anesthesia using the Xenogen IVIS system. Image acquisition was carried out with the field of view at D, subject height of 1.5 cm, and medium binning level with automatic exposure time determined by the Living Image Software. BLI signals were extracted using Living Image software: regions of interest were drawn around each tumor mass and photon intensities were recorded as total flux (photons/second—p/s).

BCMA x CD3 plus Control 1 provides modest anti-tumor efficacy, with mean BLI readings reduced slightly compared to controls. The combination of GPRC5D x CD28 bispecific antibodies (bsAb17372 or bsAb17375) and BCMA x CD3 bispecific antibody results in mean BLI readings that are lower than BCMA x CD3 plus Control 1.

These studies demonstrate that while monotherapy with BCMA x CD3 bispecific antibody (REGN5458) demonstrates modest anti-tumor efficacy, combination treatment with BCMA x CD3 bispecific antibody (REGN5458) plus GPRC5D x CD28 (bsAb17372 or bsAb17375) results in more potent, combinatorial anti-tumor efficacy that is superior to either therapy alone.

TABLE 18A
Tumor Volume at Day 12

| | Tumor Burden - | Total Flux | Number of |
|---|---|---|---|
| | Mean Total Flux | SEM on | mice still |
| Antibody Treatment | (p/s) on Day 12 | Day 12 | alive on day 12 |
| PBS | 3.00E+06 | 4.68E+05 | 5 of 5 |
| Control 2 (1 mg/kg) + Control 1 (4 mg/kg) | 1.51E+06 | 1.77E+05 | 5 of 5 |
| BCMA × CD3 bsAb (1 mg/kg) + Control 1 | 1.31E+06 | 9.54E+04 | 5 of 5 |
| (4 mg/kg) | | | |
| BCMA × CD3 bsAb (1 mg/kg) + | 9.88E+05 | 3.87E+04 | 5 of 5 |
| GPRC5D × CD28 bsAb17372 (4 mg/kg) | | | |
| BCMA × CD3 bsAb (1 mg/kg) + | 1.05E+06 | 4.37E+04 | 5 of 5 |
| GPRC5D × CD28 bsAb17375 (4 mg/kg) | | | |
| No Tumor Control | 9.72E+05 | 3.81E+04 | 5 of 5 |

TABLE 18B
Tumor Volume at Day 15

| | Tumor Burden - | Total Flux | Number of |
|---|---|---|---|
| | Mean Total Flux | SEM on | mice still |
| Antibody Treatment | (p/s) on Day 15 | Day 15 | alive on day 15 |
| PBS | 1.35E+07 | 2.81E+06 | 5 of 5 |
| Control 2 (1 mg/kg) + Control 1 (4 mg/kg) | 4.82E+06 | 5.94E+05 | 5 of 5 |
| BCMA × CD3 bsAb (1 mg/kg) + Control 1 | 3.67E+06 | 3.97E+05 | 5 of 5 |
| (4 mg/kg) | | | |
| BCMA × CD3 bsAb (1 mg/kg) + | 1.23E+06 | 9.94E+04 | 5 of 5 |
| GPRC5D × CD28 bsAb17372 (4 mg/kg) | | | |
| BCMA × CD3 bsAb (1 mg/kg) + | 1.41E+06 | 1.47E+05 | 5 of 5 |
| GPRC5D × CD28 bsAb17375 (4 mg/kg) | | | |
| No Tumor Control | 7.78E+05 | 3.19E+04 | 5 of 5 |

TABLE 18C
Tumor Volume at Day 19

| | Tumor Burden - | Total Flux | Number of |
|---|---|---|---|
| | Mean Total Flux | SEM on | mice still |
| Antibody Treatment | (p/s) on Day 19 | Day 19 | alive on day 19 |
| PBS | 6.39E+07 | 1.83E+07 | 5 of 5 |
| Control 2 (1 mg/kg) + Control 1 (4 mg/kg) | 2.02E+07 | 3.73E+06 | 5 of 5 |
| BCMA × CD3 bsAb (1 mg/kg) + Control 1 | 1.24E+07 | 1.74E+06 | 5 of 5 |
| (4 mg/kg) | | | |
| BCMA × CD3 bsAb (1 mg/kg) + | 3.37E+06 | 7.19E+05 | 5 of 5 |
| GPRC5D × CD28 bsAb17372 (4 mg/kg) | | | |
| BCMA × CD3 bsAb (1 mg/kg) + | 5.27E+06 | 4.89E+05 | 5 of 5 |
| GPRC5D × xCD28 bsAb17375 (4 mg/kg) | | | |
| No Tumor Control | 9.12E+05 | 1.30E+04 | 5 of 5 |

TABLE 18D
Tumor Volume at Day 22

| | Tumor Burden - | Total Flux | Number of |
|---|---|---|---|
| | Mean Total Flux | SEM on | mice still |
| Antibody Treatment | (p/s) on Day 22 | Day 22 | alive on day 22 |
| PBS | 1.33E+08 | 3.70E+07 | 5 of 5 |
| Control 2 (1 mg/kg) + Control 1 bsAb | 4.38E+07 | 1.36E+07 | 4 of 5 |
| (4 mg/kg) | | | |
| BCMA × CD3 bsAb (1 mg/kg) + Control 1 | 2.38E+07 | 5.28E+06 | 5 of 5 |
| (4 mg/kg) | | | |
| BCMA × CD3 bsAb (1 mg/kg) + | 4.99E+06 | 1.08E+06 | 5 of 5 |
| GPRC5D × CD28 bsAb17372 (4 mg/kg) | | | |
| BCMA × CD3 bsAb (1 mg/kg) + | 9.03E+06 | 1.14E+06 | 5 of 5 |
| GPRC5D × CD28 bsAb17375 (4 mg/kg) | | | |
| No Tumor Control | 6.12E+05 | 2.25E+04 | 5 of 5 |

TABLE 18E
Tumor Volume at Day 26

| | Tumor Burden - | Total Flux | Number of |
|---|---|---|---|
| | Mean Total Flux | SEM on | mice still |
| Antibody Treatment | (p/s) on Day 26 | Day 26 | alive on day 26 |
| PBS | 9.94E+07 | | 1 of 5 |
| Control 2 (1 mg/kg) + Control 1 (4 mg/kg) | 6.91E+07 | 2.22E+07 | 4 of 5 |
| BCMA × CD3 bsAb (1 mg/kg) + Control 1 | 3.51E+07 | 2.34E+06 | 4 of 5 |
| (4 mg/kg) | | | |
| BCMA × CD3 bsAb (1 mg/kg) + | 7.11E+06 | 8.49E+05 | 5 of 5 |
| GPRC5D × CD28 bsAb17372 (4 mg/kg) | | | |
| BCMA × CD3 bsAb (1 mg/kg) + | 1.81E+07 | 4.12E+06 | 5 of 5 |
| GPRC5D × CD28 bsAb17375 (4 mg/kg) | | | |
| No Tumor Control | 7.17E+05 | 2.21E+04 | 5 of 5 |

TABLE 18F
Tumor Volume at Day 29

| | Tumor Burden - | Total Flux | Number of |
|---|---|---|---|
| | Mean Total Flux | SEM on | mice still |
| Antibody Treatment | (p/s) on Day 29 | Day 29 | alive on day 29 |
| PBS | | | 0 of 5 |
| Control 2 (1 mg/kg) + Control 1 (4 mg/kg) | | | 0 of 5 |
| BCMA × CD3 bsAb (1 mg/kg) + Control 1 | | | 0 of 5 |
| (4 mg/kg) | | | |
| BCMA × CD3 bsAb (1 mg/kg) + | 1.61E+07 | 3.15E+06 | 3 of 5 |
| GPRC5D × CD28 bsAb17372 (4 mg/kg) | | | |
| BCMA × CD3 bsAb (1 mg/kg) + | 4.88E+07 | 2.40E+07 | 3 of 5 |
| GPRC5D × CD28 bsAb17375 (4 mg/kg) | | | |
| No Tumor Control | 9.32E+05 | 1.48E+04 | 5 of 5 |

TABLE 18G
Tumor Volume at Day 34

| | Tumor Burden - | Total Flux | Number of |
|---|---|---|---|
| | Mean Total Flux | SEM on | mice still |
| Antibody Treatment | (p/s) on Day 34 | Day 34 | alive on day 34 |
| PBS | | | 0 of 5 |
| Control 2 (1 mg/kg) + Control 1 (4 mg/kg) | | | 0 of 5 |
| BCMA × CD3 bsAb (1 mg/kg) + Control 1 | | | 0 of 5 |
| (4 mg/kg) | | | |
| BCMA × CD3 bsAb (1 mg/kg) + | 3.16E+07 | | 1 of 5 |
| GPRC5D × CD28 bsAb17372 (4 mg/kg) | | | |
| BCMA × CD3 bsAb (1 mg/kg) + | 1.41E+08 | | 1 of 5 |
| GPRC5D × CD28 bsAb17375 (4 mg/kg) | | | |
| No Tumor Control | 6.03E+05 | 4.35E+04 | 5 of 5 |

TABLE 18H
Tumor Volume at Day 37

| | Tumor Burden - | Total Flux | Number of |
|---|---|---|---|
| | Mean Total Flux | SEM on | mice still |
| Antibody Treatment | (p/s) on Day 37 | Day 37 | alive on day 37 |
| PBS | | | 0 of 5 |
| Control 2 (1 mg/kg) + Control 1 (4 mg/kg) | | | 0 of 5 |
| BCMA × CD3 bsAb (1 mg/kg) + Control 1 | | | 0 of 5 |
| (4 mg/kg) | | | |
| BCMA × CD3 bsAb (1 mg/kg) + | 6.14E+07 | | 1 of 5 |
| GPRC5D × CD28 bsAb17372 (4 mg/kg) | | | |
| BCMA × CD3 bsAb (1 mg/kg) + | | | 0 of 5 |
| GPRC5D × CD28 bsAb17375 (4 mg/kg) | | | |
| No Tumor Control | 6.52E+05 | 2.26E+04 | 5 of 5 |

### Example 9: In Vivo Antitumor-Efficacy of Bispecific Anti-GPRC5D x CD28 Antibodies in a Xenogenic Tumor Study (OPM-2 Cells)

To determine the in vivo anti-tumor efficacy of GPRC5D x CD28 bispecific antibodies (bsAb) in combination with a BCMA x CD3 bispecific antibody, a xenogenic tumor study was performed. On day 0, 5×10⁶ BCMA⁺GPRC5D⁺OPM-2 human multiple myeloma tumor cells that were engineered to also express firefly luciferase (OPM-2-luciferase cells) were intravenously administered into immunodeficient NOD.Cg-Prkdc^{scid}∥2rg^{tm1Wjl}/SzJ (NSG) mice. On day 11 the mice were intraperitoneally injected with 4×10⁶ human peripheral blood mononuclear cells (PBMC) from a normal, healthy donor. On day 21 mice were randomized for tumor burden and PBMC engraftment. The mice (n=5 per group) then received either Control 2 or a BCMA x CD3 (REGN5458) bispecific antibody at 0.04 mg/kg, in combination with Control 1, or GPRC5D x CD28 bispecific antibody (either bsAb17372 or bsAb17375) at 4 mg/kg, 0.4 mg/kg or 0.04 mg/kg. The mice were administered these antibodies twice more on days 29 and 36, for a total of three doses. Tumor growth was assessed over 52 days by measuring tumor bioluminescence (BLI) in anesthetized animals. As a positive control, a group of mice (n=5) was given only OPM-2-luciferase cells and PBMCs, but not antibody (PBS-treated group). In order to measure background BLI levels, a group of mice (n=5) were untreated and did not receive tumors, PBMC, or antibody (No Tumor group).

On day 0, 5×10⁶ BCMA⁺GPRC5D⁺OPM-2 human multiple myeloma tumor cells that were engineered to also express firefly luciferase (OPM-2-luciferase cells) were intravenously administered into immunodeficient NOD.Cg-Prkdc^{scid}∥2rg^{tm1Wjl}/SzJ (NSG) mice. On day 11 the mice were intraperitoneally injected with 4×10⁶ human peripheral blood mononuclear cells (PBMC) from a normal, healthy donor. On day 21 mice were randomized for tumor burden and PBMC engraftment. The mice (n=5 per group) then received either Control 2 or a BCMA x CD3 (REGN5458) bispecific antibody at 0.04 mg/kg, in combination with Control 1, or GPRC5D x CD28 bispecific antibody (either bsAb17372 or bsAb17375) at 4 mg/kg, 0.4 mg/kg or 0.04 mg/kg. The mice were administered these antibodies twice more on days 29 and 36, for a total of three doses. Tumor growth was assessed over 52 days by measuring tumor bioluminescence (BLI) in anesthetized animals. As a positive control, a group of mice (n=5) was given only OPM-2-luciferase cells and PBMCs, but not antibody (PBS-treated group). In order to measure background BLI levels, a group of mice (n=5) were untreated and did not receive tumors, PBMC, or antibody (No Tumor group).

BLI imaging was used to measure tumor burden. Mice were injected IP with 150 mg/kg of the luciferase substrate D-luciferin suspended in PBS. Five minutes after this injection, BLI imaging of the mice was performed under isoflurane anesthesia using the Xenogen IVIS system. Image acquisition was carried out with the field of view at D, subject height of 1.5 cm, and medium binning level with automatic exposure time determined by the Living Image Software. BLI signals were extracted using Living Image software: regions of interest were drawn around each tumor mass and photon intensities were recorded as total flux (photons/second—p/s).

Monotherapy of BCMA x CD3 (REGN5458) bispecific antibody provides only modest anti-tumor efficacy at a dose of 0.04 mg/kg, with mean BLI readings slightly lower compared to controls. The combination of GPRC5D x CD28 bispecific antibody (bsAb17372) and BCMA x CD3 bispecific antibody (REGN5458) results in enhanced tumor reduction compared to BCMA x CD3 bispecific antibody plus Control 1 at the 4 mg/kg and 0.4 mg/kg doses. Similarly, the combination of GPRC5D x CD28 bispecific antibody (bsAb17375) and BCMA x CD3 bispecific antibody (REGN5458) results in enhanced tumor reduction compared to BCMA x CD3 bispecific antibody plus Control 1 at the 4 mg/kg and 0.4 mg/kg doses.

These studies demonstrate that while monotherapy with BCMA x CD3 bispecific antibody has modest anti-tumor efficacy, combination treatment with BCMAxCD3 bispecific antibody (REGN5458) plus GPRC5D x CD28 bispecific antibody (bsAb17372 or bsAb17375) results in more potent, combinatorial anti-tumor efficacy at doses of 4 mg/kg and 0.4 mg/kg. bsAb17372 led to higher survival than bsAb17375.

TABLE 19A
Tumor Volume at Day 18

| | Tumor Burden - | Total Flux | Number of |
|---|---|---|---|
| | Mean Total Flux | SEM on | mice still |
| Antibody Treatment | (p/s) on Day 18 | Day 18 | alive on day 18 |
| Control 2 (0.04 mg/kg) + Control 1 (4 mg/kg) | 1.53E+06 | 1.82E+05 | 5 of 5 |
| BCMA × CD3 bsAb (0.04 mg/kg) + Control 1 | 1.42E+06 | 1.17E+05 | 5 of 5 |
| (4 mg/kg) | | | |
| BCMA × CD3 bsAb (0.04 mg/kg) + | 1.31E+06 | 1.13E+05 | 5 of 5 |
| GPRC5D × CD28 bsAb17372 (4 mg/kg) | | | |
| BCMA × CD3 bsAb (0.04 mg/kg) + | 1.41E+06 | 7.95E+04 | 5 of 5 |
| GPRC5D × CD28 bsAb17372 (0.4 mg/kg) | | | |
| BCMA × CD3 bsAb (0.04 mg/kg) + | 1.27E+06 | 1.35E+05 | 5 of 5 |
| GPRC5D × CD28 bsAb17372 (0.04 mg/kg) | | | |
| BCMA × CD3 bsAb (0.04 mg/kg) + | 1.29E+06 | 6.37E+04 | 5 of 5 |
| GPRC5D × CD28 bsAb17375 (4 mg/kg) | | | |
| BCMA × CD3 bsAb (0.04 mg/kg) + | 8.49E+05 | 3.45E+04 | 5 of 5 |
| GPRC5D × CD28 bsAb17375 (0.4 mg/kg) | | | |
| BCMA × CD3 bsAb (0.04 mg/kg) + | 1.34E+06 | 3.33E+04 | 5 of 5 |
| GPRC5D × CD28 bsAb17375 (0.04 mg/kg) | | | |
| No Tumor (Background BLI) | 9.92E+05 | 5.31E+04 | 4 of 4 |

TABLE 19B
Tumor Volume at Day 24

| | Tumor Burden - | Total Flux | Number of |
|---|---|---|---|
| | Mean Total Flux | SEM on | mice still |
| Antibody Treatment | (p/s) on Day 24 | Day 24 | alive on day 24 |
| Control 2 (0.04 mg/kg) + Control 1 (4 mg/kg) | 4.47E+06 | 9.93E+05 | 5 of 5 |
| BCMA × CD3 bsAb (0.04 mg/kg) + Control 1 | 2.80E+06 | 3.25E+05 | 5 of 5 |
| (4 mg/kg) | | | |
| BCMA × CD3 bsAb (0.04 mg/kg) + | 3.84E+06 | 2.97E+05 | 5 of 5 |
| GPRC5D × CD28 bsAb17372 (4 mg/kg) | | | |
| BCMA × CD3 bsAb (0.04 mg/kg) + | 2.31E+06 | 2.16E+05 | 5 of 5 |
| GPRC5D × CD28 bsAb17372 (0.4 mg/kg) | | | |
| BCMA × CD3 bsAb (0.04 mg/kg) + | 2.87E+06 | 4.42E+05 | 5 of 5 |
| GPRC5D × CD28 bsAb17372 (0.04 mg/kg) | | | |
| BCMA × CD3 bsAb (0.04 mg/kg) + | 2.32E+06 | 3.09E+05 | 5 of 5 |
| GPRC5D × CD28 bsAb17375 (4 mg/kg) | | | |
| BCMA × CD3 bsAb (0.04 mg/kg) + | 1.33E+06 | 2.07E+05 | 5 of 5 |
| GPRC5D × CD28 bsAb17375 (0.4 mg/kg) | | | |
| BCMA × CD3 bsAb (0.04 mg/kg) + | 3.35E+06 | 4.79E+05 | 5 of 5 |
| GPRC5D × CD28 bsAb17375 (0.04 mg/kg) | | | |
| No Tumor (Background BLI) | 6.98E+05 | 3.20E+04 | 4 of 4 |

TABLE 19C
Tumor Volume at Day 29

| | Tumor Burden - | Total Flux | Number of |
|---|---|---|---|
| | Mean Total Flux | SEM on | mice still |
| Antibody Treatment | (p/s) on Day 29 | Day 29 | alive on day 29 |
| Control 2 (0.04 mg/kg) + Control 1 (4 mg/kg) | 9.61E+06 | 1.02E+06 | 5 of 5 |
| BCMA × CD3 bsAb (0.04 mg/kg) + Control 1 | 5.29E+06 | 1.14E+06 | 5 of 5 |
| (4 mg/kg) | | | |
| BCMA × CD3 bsAb (0.04 mg/kg) + | 4.46E+06 | 1.14E+06 | 5 of 5 |
| GPRC5D × CD28 bsAb17372 (4 mg/kg) | | | |
| BCMA × CD3 bsAb (0.04 mg/kg) + | 2.30E+06 | 5.17E+05 | 5 of 5 |
| GPRC5D × CD28 bsAb17372 (0.4 mg/kg) | | | |
| BCMA × CD3 bsAb (0.04 mg/kg) + | 5.45E+06 | 9.09E+05 | 5 of 5 |
| GPRC5D × CD28 bsAb17372 (0.04 mg/kg) | | | |
| BCMA × CD3 bsAb (0.04 mg/kg) + | 2.63E+06 | 7.69E+05 | 5 of 5 |
| GPRC5D × CD28 bsAb17375 (4 mg/kg) | | | |
| BCMA × CD3 bsAb (0.04 mg/kg) + | 1.55E+06 | 5.52E+05 | 5 of 5 |
| GPRC5D × CD28 bsAb17375 (0.4 mg/kg) | | | |
| BCMA × CD3 bsAb (0.04 mg/kg) + | 5.94E+06 | 1.54E+06 | 5 of 5 |
| GPRC5D × CD28 bsAb17375 (0.04 mg/kg) | | | |
| No Tumor (Background BLI) | 8.43E+05 | 1.45E+04 | 4 of 4 |

TABLE 19D
Tumor Volume at Day 32

| | Tumor Burden - | Total Flux | Number of |
|---|---|---|---|
| | Mean Total Flux | SEM on | mice still |
| Antibody Treatment | (p/s) on Day 32 | Day 32 | alive on day 32 |
| Control 2 (0.04 mg/kg) + Control 1 (4 mg/kg) | 1.89E+07 | 1.16E+06 | 5 of 5 |
| BCMA × CD3 bsAb (0.04 mg/kg) + Control 1 | 7.05E+06 | 1.43E+06 | 5 of 5 |
| (4 mg/kg) | | | |
| BCMA × CD3 bsAb (0.04 mg/kg) + | 3.38E+06 | 1.31E+06 | 5 of 5 |
| GPRC5D × CD28 bsAb17372 (4 mg/kg) | | | |
| BCMA × CD3 bsAb (0.04 mg/kg) + | 2.67E+06 | 7.56E+05 | 5 of 5 |
| GPRC5D × CD28 bsAb17372 (0.4 mg/kg) | | | |
| BCMA × CD3 bsAb (0.04 mg/kg) + | 7.54E+06 | 1.91E+06 | 5 of 5 |
| GPRC5D × CD28 bsAb17372 (0.04 mg/kg) | | | |
| BCMA × CD3 bsAb (0.04 mg/kg) + | 2.14E+06 | 6.56E+05 | 5 of 5 |
| GPRC5D × CD28 bsAb17375 (4 mg/kg) | | | |
| BCMA × CD3 bsAb (0.04 mg/kg) + | 3.36E+06 | 2.49E+06 | 5 of 5 |
| GPRC5D × CD28 bsAb17375 (0.4 mg/kg) | | | |
| BCMA × CD3 bsAb (0.04 mg/kg) + | 8.54E+06 | 2.13E+06 | 5 of 5 |
| GPRC5D × CD28 bsAb17375 (0.04 mg/kg) | | | |
| No Tumor (Background BLI) | 7.66E+05 | 6.78E+04 | 4 of 4 |

TABLE 19E
Tumor Volume at Day 39

| | Tumor Burden - | Total Flux | Number of |
|---|---|---|---|
| | Mean Total Flux | SEM on | mice still |
| Antibody Treatment | (p/s) on Day 39 | Day 39 | alive on day 39 |
| Control 2 (0.04 mg/kg) + Control 1 (4 mg/kg) | 3.20E+07 | | 1 of 5 |
| BCMA × CD3 bsAb (0.04 mg/kg) + Control 1 | 1.10E+07 | 4.17E+06 | 3 of 5 |
| (4 mg/kg) | | | |
| BCMA × CD3 bsAb (0.04 mg/kg) + | 4.48E+06 | 2.37E+06 | 5 of 5 |
| GPRC5D × CD28 bsAb17372 (4 mg/kg) | | | |
| BCMA × CD3 bsAb (0.04 mg/kg) + | 2.38E+06 | 1.10E+06 | 5 of 5 |
| GPRC5D × CD28 bsAb17372 (0.4 mg/kg) | | | |
| BCMA × CD3 bsAb (0.04 mg/kg) + | 8.45E+06 | | 1 of 5 |
| GPRC5D × CD28 bsAb17372 (0.04 mg/kg) | | | |
| BCMA × CD3 bsAb (0.04 mg/kg) + | 1.43E+06 | 3.12E+05 | 4 of 5 |
| GPRC5D × CD28 bsAb17375 (4 mg/kg) | | | |
| BCMA × CD3 bsAb (0.04 mg/kg) + | 4.06E+06 | 3.43E+06 | 5 of 5 |
| GPRC5D × CD28 bsAb17375 (0.4 mg/kg) | | | |
| BCMA × CD3 bsAb (0.04 mg/kg) + | 1.63E+07 | 1.49E+07 | 2 of 5 |
| GPRC5D × CD28 bsAb17375 (0.04 mg/kg) | | | |
| No Tumor (Background BLI) | 6.95E+05 | 1.36E+04 | 4 of 4 |

TABLE 19F
Tumor Volume at Day 42

| | Tumor Burden - | | Number of mice |
|---|---|---|---|
| Antibody | Mean Total Flux | Total Flux SEM | still alive |
| Treatment | (p/s) on Day 42 | on Day 42 | on day 42 |
| Control 2 (0.04 mg/kg) + Control 1 (4 mg/kg) | | | 0 of 5 |
| BCMA × CD3 bsAb (0.04 mg/kg) + Control 1 | 1.68E+07 | 5.50E+06 | 3 of 5 |
| (4 mg/kg) | | | |
| BCMA × CD3 bsAb (0.04 mg/kg) + | 3.99E+06 | 2.41E+06 | 5 of 5 |
| GPRC5D × CD28 bsAb17372 (4 mg/kg) | | | |
| BCMA × CD3 bsAb (0.04 mg/kg) + | 3.54E+06 | 1.38E+06 | 5 of 5 |
| GPRC5D × CD28 bsAb17372 (0.4 mg/kg) | | | |
| BCMA × CD3 bsAb (0.04 mg/kg) + | | | 0 of 5 |
| GPRC5D × CD28 bsAb17372 (0.04 mg/kg) | | | |
| BCMA × CD3 bsAb (0.04 mg/kg) + | 1.50E+06 | 5.00E+05 | 4 of 5 |
| GPRC5D × CD28 bsAb17375 (4 mg/kg) | | | |
| BCMA × CD3 bsAb (0.04 mg/kg) + | 6.16E+06 | 5.24E+06 | 5 of 5 |
| GPRC5D × CD28 bsAb17375 (0.4 mg/kg) | | | |
| BCMA × CD3 bsAb (0.04 mg/kg) + | 2.52E+07 | 2.37E+07 | 2 of 5 |
| GPRC5D × CD28 bsAb17375 (0.04 mg/kg) | | | |
| No Tumor (Background BLI) | 6.83E+05 | 1.82E+04 | 4 of 4 |

### Example 10: In Vivo Antitumor-Efficacy of Bispecific Anti-GPRC5D x CD28 Antibodies in a Xenogenic Tumor Study (OPM-2 Cells)

To determine the in vivo anti-tumor efficacy of GPRC5DxCD28 bispecific antibodies (bsAb) in combination with a BCMAxCD3 bispecific antibody, a study was performed in immunocompetent C57BL/6 mice that transgenically express human BCMA, GPRC5D, CD3, and CD28 in place of the murine versions of these genes (BCMA/GPRC5D/CD3/CD28-humanized mice). On Day 0, BCMA/GPRC5D/CD3/CD28-humanized mice were subcutaneously injected with 1×10⁶ MC38 tumor cells engineered to express human BCMA and human GPRC5D (MC38/hBCMA/hGPRC5D cells). On Day 4, the mice (n=6-7 per group) were administered either a CD3-binding control bispecific antibody (H4sH17664D) or a BCMAxCD3 (REGN5458) bispecific antibody at 1 mg/kg, in combination with a CD28-binding control bispecific antibody (REGN6157), or a GPRC5DxCD28 bispecific antibody (REGN17372) at 4 mg/kg, 0.4 mg/kg or 0.04 mg/kg. The mice were administered these antibodies three more times on days 7, 11, and 14, for a total of four doses. Tumor growth was assessed over 21 days twice weekly by using calipers and calculated by the following formula: (length×width²)/2.

BCMAxCD3 bispecific antibody (REGN5458) plus CD28-binding control bispecific antibody (REGN6157) provides very modest anti-tumor efficacy compared to CD3-binding control bispecific antibody (H4sH17664D) plus CD28-binding control bispecific antibody (REGN6157). The combination of BCMAxCD3 bispecific antibody (REGN5458) plus GPRC5DxCD28 bispecific antibody (REGN17372) results in mean tumor volumes that are significantly lower than mice receiving BCMAxCD3 bispecific antibody (REGN5458) plus CD28-binding control bispecific antibody (REGN6157) at 4 mg/kg (p=0.0008 at day 18 and p<0.0001 at day 21, by 2-way ANOVA with tukey's multiple comparisons test).

These studies demonstrate that while monotherapy with BCMAxCD3 bispecific antibody shows very modest anti-tumor efficacy, combination treatment with BCMAxCD3 bispecific antibody (REGN5458) plus GPRC5DxCD28 bispecific antibody (REGN17372) results in more potent, combinatorial anti-tumor efficacy that is superior to BCMAxCD3 bispecific antibody (REGN5458) plus CD28-binding control bispecific antibody (REGN6157).

TABLE 20A
Tumor Volume at Day 4

| | Mean Tumor | Tumor | Number of |
|---|---|---|---|
| Antibody | Volume (mm3) | Volume SEM | mice still |
| Treatment | on Day 4 | on Day 4 | alive on day 4 |
| CD3-binding control bsAb (1 mg/kg) + CD28- | 17.7 | 3.7 | 6 of 6 |
| binding control bsAb (4 mg/kg) | | | |
| CD3-binding control bsAb (1 mg/kg) + | 16.2 | 2.7 | 6 of 6 |
| GPRC5D × CD28 REGN17372 (4 mg/kg) | | | |
| BCMA × CD3 bsAb (1 mg/kg) + CD28-binding | 17.0 | 4.7 | 7 of 7 |
| control bsAb (4 mg/kg) | | | |
| BCMA × CD3 bsAb (1 mg/kg) + GPRC5D × CD28 | 11.0 | 2.4 | 7 of 7 |
| REGN17372 (4 mg/kg) | | | |
| BCMA × CD3 bsAb (1 mg/kg) + GPRC5D × CD28 | 15.3 | 0.9 | 7 of 7 |
| REGN17372 (0.4 mg/kg) | | | |
| BCMA × CD3 bsAb (1 mg/kg) + GPRC5D × CD28 | 15.7 | 4.8 | 7 of 7 |
| REGN17372 (0.04 mg/kg) | | | |

TABLE 20B
Tumor Volume at Day 7

| | Mean Tumor | Tumor | Number of |
|---|---|---|---|
| Antibody | Volume (mm3) | Volume SEM | mice still |
| Treatment | on Day 7 | on Day 7 | alive on day 7 |
| CD3-binding control bsAb (1 mg/kg) + CD28- | 55.5 | 10.4 | 6 of 6 |
| binding control bsAb (4 mg/kg) | | | |
| CD3-binding control bsAb (1 mg/kg) + | 47.7 | 10.2 | 6 of 6 |
| GPRC5D × CD28 REGN17372 (4 mg/kg) | | | |
| BCMA × CD3 bsAb (1 mg/kg) + CD28-binding | 27.6 | 10.1 | 7 of 7 |
| control bsAb (4 mg/kg) | | | |
| BCMA × CD3 bsAb (1 mg/kg) + GPRC5D × CD28 | 14.3 | 4.2 | 7 of 7 |
| REGN17372 (4 mg/kg) | | | |
| BCMA × CD3 bsAb (1 mg/kg) + GPRC5D × CD28 | 22.3 | 6.9 | 7 of 7 |
| REGN17372 (0.4 mg/kg) | | | |
| BCMA × CD3 bsAb (1 mg/kg) + GPRC5D × CD28 | 24.4 | 8.2 | 7 of 7 |
| REGN17372 (0.04 mg/kg) | | | |

TABLE 20C
Tumor Volume at Day 11

| | Mean Tumor | Tumor | Number of |
|---|---|---|---|
| Antibody | Volume (mm3) | Volume SEM | mice still |
| Treatment | on Day 11 | on Day 11 | alive on day 11 |
| CD3-binding control bsAb (1 mg/kg) + CD28- | 261.3 | 35.5 | 6 of 6 |
| binding control bsAb (4 mg/kg) | | | |
| CD3-binding control bsAb (1 mg/kg) + | 146.2 | 26.3 | 6 of 6 |
| GPRC5D × CD28 REGN17372 (4 mg/kg) | | | |
| BCMA × CD3 bsAb (1 mg/kg) + CD28-binding | 77.0 | 33.7 | 7 of 7 |
| control bsAb (4 mg/kg) | | | |
| BCMA × CD3 bsAb (1 mg/kg) + GPRC5D × CD28 | 20.1 | 10.3 | 7 of 7 |
| REGN17372 (4 mg/kg) | | | |
| BCMA × CD3 bsAb (1 mg/kg) + GPRC5D × CD28 | 64.0 | 24.6 | 7 of 7 |
| REGN17372 (0.4 mg/kg) | | | |
| BCMA × CD3 bsAb (1 mg/kg) + GPRC5D × CD28 | 43.3 | 14.4 | 7 of 7 |
| REGN17372 (0.04 mg/kg) | | | |

TABLE 20D
Tumor Volume at Day 14

| | Mean Tumor | Tumor | Number of |
|---|---|---|---|
| Antibody | Volume (mm3) | Volume SEM | mice still |
| Treatment | on Day 14 | on Day 14 | alive on day 14 |
| CD3-binding control bsAb (1 mg/kg) + CD28- | 512.2 | 49.8 | 6 of 6 |
| binding control bsAb (4 mg/kg) | | | |
| CD3-binding control bsAb (1 mg/kg) + | 272.7 | 60.9 | 6 of 6 |
| GPRC5D × CD28 REGN17372 (4 mg/kg) | | | |
| BCMA × CD3 bsAb (1 mg/kg) + CD28-binding | 188.4 | 73.6 | 7 of 7 |
| control bsAb (4 mg/kg) | | | |
| BCMA × CD3 bsAb (1 mg/kg) + GPRC5D × CD28 | 57.7 | 35.4 | 7 of 7 |
| REGN17372 (4 mg/kg) | | | |
| BCMA × CD3 bsAb (1 mg/kg) + GPRC5D × CD28 | 218.1 | 101.9 | 7 of 7 |
| REGN17372 (0.4 mg/kg) | | | |
| BCMA × CD3 bsAb (1 mg/kg) + GPRC5D × CD28 | 154.9 | 56.9 | 7 of 7 |
| REGN17372 (0.04 mg/kg) | | | |

TABLE 20E
Tumor Volume at Day 18

| | Mean Tumor | Tumor | Number of |
|---|---|---|---|
| Antibody | Volume (mm3) | Volume SEM | mice still |
| Treatment | on Day 18 | on Day 18 | alive on day 18 |
| CD3-binding control bsAb (1 mg/kg) + CD28- | 1430.7 | 123.1 | 6 of 6 |
| binding control bsAb (4 mg/kg) | | | |
| CD3-binding control bsAb (1 mg/kg) + | 813.4 | 150.8 | 5 of 6 |
| GPRC5D × CD28 REGN17372 (4 mg/kg) | | | |
| BCMA × CD3 bsAb (1 mg/kg) + CD28-binding | 606.6 | 207.8 | 7 of 7 |
| control bsAb (4 mg/kg) | | | |
| BCMA × CD3 bsAb (1 mg/kg) + GPRC5D × CD28 | 124.9 | 113.3 | 7 of 7 |
| REGN17372 (4 mg/kg) | | | |
| BCMA × CD3 bsAb (1 mg/kg) + GPRC5D × CD28 | 640.3 | 295.6 | 7 of 7 |
| REGN17372 (0.4 mg/kg) | | | |
| BCMA × CD3 bsAb (1 mg/kg) + GPRC5D × CD28 | 330.7 | 128.1 | 7 of 7 |
| REGN17372 (0.04 mg/kg) | | | |

TABLE 20F
Tumor Volume at Day 21

| | Mean Tumor | Tumor | Number of |
|---|---|---|---|
| Antibody | Volume (mm3) | Volume SEM | mice still |
| Treatment | on Day 21 | on Day 21 | alive on day 21 |
| CD3-binding control bsAb (1 mg/kg) + CD28- | 2597.0 | 237.8 | 4 of 6 |
| binding control bsAb (4 mg/kg) | | | |
| CD3-binding control bsAb (1 mg/kg) + | 1542.4 | 318.9 | 5 of 6 |
| GPRC5D × CD28 REGN17372 (4 mg/kg) | | | |
| BCMA × CD3 bsAb (1 mg/kg) + CD28-binding | 1203.7 | 407.0 | 7 of 7 |
| control bsAb (4 mg/kg) | | | |
| BCMA × CD3 bsAb (1 mg/kg) + GPRC5D × CD28 | 263.4 | 250.1 | 7 of 7 |
| REGN17372 (4 mg/kg) | | | |
| BCMA × CD3 bsAb (1 mg/kg) + GPRC5D × CD28 | 806.8 | 224.7 | 6 of 7 |
| REGN17372 (0.4 mg/kg) | | | |
| BCMA × CD3 bsAb (1 mg/kg) + GPRC5D × CD28 | 796.0 | 339.9 | 7 of 7 |
| REGN17372 (0.04 mg/kg) | | | |

### Example 11: In Vivo Antitumor-Efficacy of Bispecific Anti-GPRC5D x CD28 Antibodies in a Xenogenic Tumor Study (OPM-2 Cells)

To determine the in vivo anti-tumor efficacy of GPRC5DxCD28 bispecific antibodies (bsAb) in combination with a BCMAxCD3 bispecific antibody, a study was performed in immunocompetent C57BL/6 mice that transgenically express human BCMA, GPRC5D, CD3, and CD28 in place of the murine versions of these genes (BCMA/GPRC5D/CD3/CD28-humanized mice). On Day 0, BCMA/GPRC5D/CD3/CD28-humanized mice were subcutaneously injected with 1×10⁶ MC38 tumor cells engineered to express human BCMA and human GPRC5D (MC38/hBCMA/hGPRC5D cells). On Day 7, the mice (n=6 per group) were administered either a CD3-binding control bispecific antibody (H4sH17664D) or a BCMAxCD3 (REGN5458) bispecific antibody at 4 mg/kg, in combination with a CD28-binding control bispecific antibody (REGN6157), or a GPRC5DxCD28 bispecific antibody (REGN17372) at 12 mg/kg, 4 mg/kg or 1 mg/kg. The mice were administered these antibodies three more times on days 10, 14, and 17, for a total of four doses. Tumor growth was assessed over 27 days twice weekly by using calipers and calculated by the following formula: (length×width²)/2.

BCMAxCD3 bispecific antibody (REGN5458) plus CD28-binding control bispecific antibody (REGN6157) provides modest anti-tumor efficacy compared to CD3-binding control bispecific antibody (H4sH17664D) plus CD28-binding control bispecific antibody (REGN6157). The combination of BCMAxCD3 bispecific antibody (REGN5458) plus GPRC5DxCD28 bispecific antibody (REGN17372) results in mean tumor volumes that are significantly lower than mice receiving BCMAxCD3 bispecific antibody (REGN5458) plus CD28-binding control bispecific antibody (REGN6157) at all three doses tested (On day 16, p=0.0129, p=0.0302, and p=0.3238 for 12 mg/kg, 4 mg/kg, and 1 mg/kg doses, respectively, by 2-way ANOVA with tukey's multiple comparisons test; On day 20, p<0.0001 for all doses by 2-way ANOVA with tukey's multiple comparisons test).

These studies demonstrate that while monotherapy with BCMAxCD3 bispecific antibody shows very modest anti-tumor efficacy, combination treatment with BCMAxCD3 bispecific antibody (REGN5458) plus GPRC5DxCD28 bispecific antibody (REGN17372) results in more potent, combinatorial anti-tumor efficacy that is superior to BCMAxCD3 bispecific antibody (REGN5458) plus CD28-binding control bispecific antibody (REGN6157).

TABLE 21A
Tumor Volume at Day 3

| | Mean Tumor | Tumor | Number of |
|---|---|---|---|
| Antibody | Volume (mm3) | Volume SEM | mice still |
| Treatment | on Day 3 | on Day 3 | alive on day 3 |
| CD3-binding control bsAb (4 mg/kg) + CD28- | 17.2 | 4.1 | 6 of 6 |
| binding control bsAb (12 mg/kg) | | | |
| CD3-binding control bsAb (4 mg/kg) + | 25.2 | 5.4 | 6 of 6 |
| GPRC5D × CD28 REGN17372 (12 mg/kg) | | | |
| BCMA × CD3 bsAb (4 mg/kg) + CD28-binding | 25.2 | 3.4 | 6 of 6 |
| control bsAb (12 mg/kg) | | | |
| BCMA × CD3 bsAb (4 mg/kg) + GPRC5D × CD28 | 19.5 | 4.5 | 6 of 6 |
| REGN17372 (12 mg/kg) | | | |
| BCMA × CD3 bsAb (4 mg/kg) + GPRC5D × CD28 | 21.5 | 6.1 | 6 of 6 |
| REGN17372 (4 mg/kg) | | | |
| BCMA × CD3 bsAb (4 mg/kg) + GPRC5D × CD28 | 28.5 | 2.9 | 6 of 6 |
| REGN17372 (1 mg/kg) | | | |

TABLE 21B
Tumor Volume at Day 6

| | Mean Tumor | Tumor | Number of |
|---|---|---|---|
| Antibody | Volume (mm3) | Volume SEM | mice still |
| Treatment | on Day 6 | on Day 6 | alive on day 6 |
| CD3-binding control bsAb (4 mg/kg) + CD28- | 40.5 | 8.0 | 6 of 6 |
| binding control bsAb (12 mg/kg) | | | |
| CD3-binding control bsAb (4 mg/kg) + | 52.7 | 12.0 | 6 of 6 |
| GPRC5D × CD28 REGN17372 (12 mg/kg) | | | |
| BCMA × CD3 bsAb (4 mg/kg) + CD28-binding | 60.2 | 5.0 | 6 of 6 |
| control bsAb (12 mg/kg) | | | |
| BCMA × CD3 bsAb (4 mg/kg) + GPRC5D × CD28 | 42.8 | 12.7 | 6 of 6 |
| REGN17372 (12 mg/kg) | | | |
| BCMA × CD3 bsAb (4 mg/kg) + GPRC5D × CD28 | 54.2 | 11.1 | 6 of 6 |
| REGN17372 (4 mg/kg) | | | |
| BCMA × CD3 bsAb (4 mg/kg) + GPRC5D × CD28 | 59.3 | 5.2 | 6 of 6 |
| REGN17372 (1 mg/kg) | | | |

TABLE 21C
Tumor Volume at Day 9

| | Mean Tumor | Tumor | Number of |
|---|---|---|---|
| Antibody | Volume (mm3) | Volume SEM | mice still |
| Treatment | on Day 9 | on Day 9 | alive on day 9 |
| CD3-binding control bsAb (4 mg/kg) + CD28- | 122.7 | 18.6 | 6 of 6 |
| binding control bsAb (12 mg/kg) | | | |
| CD3-binding control bsAb (4 mg/kg) + | 121.5 | 24.3 | 6 of 6 |
| GPRC5D × CD28 REGN17372 (12 mg/kg) | | | |
| BCMA × CD3 bsAb (4 mg/kg) + CD28-binding | 112.3 | 21.2 | 6 of 6 |
| control bsAb (12 mg/kg) | | | |
| BCMA × CD3 bsAb (4 mg/kg) + GPRC5D × CD28 | 38.7 | 10.7 | 6 of 6 |
| REGN17372 (12 mg/kg) | | | |
| BCMA × CD3 bsAb (4 mg/kg) + GPRC5D × CD28 | 59.5 | 5.0 | 6 of 6 |
| REGN17372 (4 mg/kg) | | | |
| BCMA × CD3 bsAb (4 mg/kg) + GPRC5D × CD28 | 44.0 | 6.5 | 6 of 6 |
| REGN17372 (1 mg/kg) | | | |

TABLE 21D
Tumor Volume at Day 13

| | Mean Tumor | Tumor | Number of |
|---|---|---|---|
| Antibody | Volume (mm3) | Volume SEM | mice still |
| Treatment | on Day 13 | on Day 13 | alive on day 13 |
| CD3-binding control bsAb (4 mg/kg) + CD28- | 289.7 | 37.0 | 6 of 6 |
| binding control bsAb (12 mg/kg) | | | |
| CD3-binding control bsAb (4 mg/kg) + | 314.8 | 50.2 | 6 of 6 |
| GPRC5D × CD28 REGN17372 (12 mg/kg) | | | |
| BCMA × CD3 bsAb (4 mg/kg) + CD28-binding | 172.2 | 49.8 | 6 of 6 |
| control bsAb (12 mg/kg) | | | |
| BCMA × CD3 bsAb (4 mg/kg) + GPRC5D × CD28 | 1.7 | 1.7 | 6 of 6 |
| REGN17372 (12 mg/kg) | | | |
| BCMA × CD3 bsAb (4 mg/kg) + GPRC5D × CD28 | 24.8 | 9.8 | 6 of 6 |
| REGN17372 (4 mg/kg) | | | |
| BCMA × CD3 bsAb (4 mg/kg) + GPRC5D × CD28 | 19.0 | 10.9 | 6 of 6 |
| REGN17372 (1 mg/kg) | | | |

TABLE 21E
Tumor Volume at Day 16

| | Mean Tumor | Tumor | Number of |
|---|---|---|---|
| Antibody | Volume (mm3) | Volume SEM | mice still |
| Treatment | on Day 16 | on Day 16 | alive on day 16 |
| CD3-binding control bsAb (4 mg/kg) + CD28- | 602.3 | 81.7 | 6 of 6 |
| binding control bsAb (12 mg/kg) | | | |
| CD3-binding control bsAb (4 mg/kg) + | 596.2 | 74.0 | 6 of 6 |
| GPRC5D × CD28 REGN17372 (12 mg/kg) | | | |
| BCMA × CD3 bsAb (4 mg/kg) + CD28-binding | 257.3 | 83.2 | 6 of 6 |
| control bsAb (12 mg/kg) | | | |
| BCMA × CD3 bsAb (4 mg/kg) + GPRC5D × CD28 | 2.8 | 2.8 | 6 of 6 |
| REGN17372 (12 mg/kg) | | | |
| BCMA × CD3 bsAb (4 mg/kg) + GPRC5D × CD28 | 27.3 | 14.1 | 6 of 6 |
| REGN17372 (4 mg/kg) | | | |
| BCMA × CD3 bsAb (4 mg/kg) + GPRC5D × CD28 | 29.8 | 23.8 | 6 of 6 |
| REGN17372 (1 mg/kg) | | | |

TABLE 21F
Tumor Volume at Day 20

| | Mean Tumor | Tumor | Number of |
|---|---|---|---|
| Antibody | Volume (mm3) | Volume SEM | mice still |
| Treatment | on Day 20 | on Day 20 | alive on day 20 |
| CD3-binding control bsAb (4 mg/kg) + CD28- | 1151.2 | 72.2 | 6 of 6 |
| binding control bsAb (12 mg/kg) | | | |
| CD3-binding control bsAb (4 mg/kg) + | 1445.8 | 168.7 | 6 of 6 |
| GPRC5D × CD28 REGN17372 (12 mg/kg) | | | |
| BCMA × CD3 bsAb (4 mg/kg) + CD28-binding | 670.8 | 253.5 | 6 of 6 |
| control bsAb (12 mg/kg) | | | |
| BCMA × CD3 bsAb (4 mg/kg) + GPRC5D × CD28 | 2.5 | 2.5 | 6 of 6 |
| REGN17372 (12 mg/kg) | | | |
| BCMA × CD3 bsAb (4 mg/kg) + GPRC5D × CD28 | 33.7 | 19.1 | 6 of 6 |
| REGN17372 (4 mg/kg) | | | |
| BCMA × CD3 bsAb (4 mg/kg) + GPRC5D × CD28 | 71.0 | 71.0 | 6 of 6 |
| REGN17372 (1 mg/kg) | | | |

TABLE 21G
Tumor Volume at Day 23

| | Mean Tumor | Tumor | Number of |
|---|---|---|---|
| Antibody | Volume (mm3) | Volume SEM | mice still |
| Treatment | on Day 23 | on Day 23 | alive on day 23 |
| CD3-binding control bsAb (4 mg/kg) + CD28- | 2115.8 | 305.4 | 5 of 6 |
| binding control bsAb (12 mg/kg) | | | |
| CD3-binding control bsAb (4 mg/kg) + | 1815.3 | 237.7 | 3 of 6 |
| GPRC5D × CD28 REGN17372 (12 mg/kg) | | | |
| BCMA × CD3 bsAb (4 mg/kg) + CD28-binding | 1067.4 | 468.5 | 5 of 6 |
| control bsAb (12 mg/kg) | | | |
| BCMA × CD3 bsAb (4 mg/kg) + GPRC5D × CD28 | 0.0 | 0.0 | 6 of 6 |
| REGN17372 (12 mg/kg) | | | |
| BCMA × CD3 bsAb (4 mg/kg) + GPRC5D × CD28 | 58.2 | 36.4 | 6 of 6 |
| REGN17372 (4 mg/kg) | | | |
| BCMA × CD3 bsAb (4 mg/kg) + GPRC5D × CD28 | 104.5 | 104.5 | 6 of 6 |
| REGN17372 (1 mg/kg) | | | |

TABLE 21H
Tumor Volume at Day 27

| | Mean Tumor | Tumor | Number of |
|---|---|---|---|
| Antibody | Volume (mm3) | Volume SEM | mice still |
| Treatment | on Day 27 | on Day 27 | alive on day 27 |
| CD3-binding control bsAb (4 mg/kg) + CD28- | | | 0 of 6 |
| binding control bsAb (12 mg/kg) | | | |
| CD3-binding control bsAb (4 mg/kg) + | | | 0 of 6 |
| GPRC5D × CD28 REGN17372 (12 mg/kg) | | | |
| BCMA × CD3 bsAb (4 mg/kg) + CD28-binding | | | 0 of 6 |
| control bsAb (12 mg/kg) | | | |
| BCMA × CD3 bsAb (4 mg/kg) + GPRC5D × CD28 | 0.0 | 0.0 | 6 of 6 |
| REGN17372 (12 mg/kg) | | | |
| BCMA × CD3 bsAb (4 mg/kg) + GPRC5D × CD28 | 144.5 | 102.0 | 6 of 6 |
| REGN17372 (4 mg/kg) | | | |
| BCMA × CD3 bsAb (4 mg/kg) + GPRC5D × CD28 | 305.3 | 305.3 | 6 of 6 |
| REGN17372 (1 mg/kg) | | | |

Sequences

SEQ ID NO: 1
caggttcagctggtgcagtctggagctgaggtgaagaagcctggggcctcagtgaaggtctcctgcaaggctt
ctggttacacctttaccagctatggtatcagctgggtgcgacaggcccctggacaaggacttgagtggatggg
atggatcagcccttacaatgttaacacaaactatgcacagaagctccagggcagagtcaccatgaccacagac
acatccacgaacacagcctacatggagctgaggagcctgagatctgacgactcggccgtgtattactgttcga
gagctgggatgccagctcgtcgctacggtatggacgtctggggccaagggaccacggtcaccgtctcctca
SEQ ID NO: 2
QVQLVQSGAEVKKPGASVKVSCKASGYTFTSYGISWVRQAPGQGLEWMGWISPYNVNTNYAQKLQGRVTMTTD
TSTNTAYMELRSLRSDDSAVYYCSRAGMPARRYGMDVWGQGTTVTVSS
SEQ ID NO: 3
ggttacacctttaccagctatggt
SEQ ID NO: 4
GYTFTSYG
SEQ ID NO: 5
atcagcccttacaatgttaacaca
SEQ ID NO: 6
ISPYNVNT
SEQ ID NO: 7
tcgagagctgggatgccagctcgtcgctacggtatggacgtc
SEQ ID NO: 8
SRAGMPARRYGMDV
SEQ ID NO: 9
caggtgcagctgcaggagtcgggcccaggactggtgaagccttcggagaccctgtccctcacctgcactgtct
ctggtggctccatcagtagttactactggagctggatccggcagcccccagggaagggactggagtggattgg
gtatatctattacagtgggatcacccactacaacccctccctcaagagtcgagtcaccatatcagtagacacg
tccaagatccagttctccctgaagctgagttctgtgaccgctgcggacacggccgtgtattactgtgcgagat
ggggggttcggagggactactactactacggtatggacgtctggggccaagggaccacggtcaccgtctcctc
a
SEQ ID NO: 10
QVQLQESGPGLVKPSETLSLTCTVSGGSISSYYWSWIRQPPGKGLEWIGYIYYSGITHYNPSLKSRVTISVDT
SKIQFSLKLSSVTAADTAVYYCARWGVRRDYYYYGMDVWGQGTTVTVSS
SEQ ID NO: 11
ggtggctccatcagtagttactac
SEQ ID NO: 12
GGSISSYY
SEQ ID NO: 13
atctattacagtgggatcacc
SEQ ID NO: 14
IYYSGIT
SEQ ID NO: 15
gcgagatggggggttcggagggactactactactacggtatggacgtc
SEQ ID NO: 16
ARWGVRRDYYYYGMDV
SEQ ID NO: 17
gaaatagttttgacacagagtcccggcacactgtcactctctcccggggaaagagccaccttgtcatgtagag
caagtcagtcagtctctagctcttatctcgcctggtaccagcagaagccgggacaggcccctagactgctgat
ctacggggcaagttccagggccaccggaatccccgaccggttcagtggaagcggaagcggaaccgattttact
ttgacgatttctagactggagccagaggatttcgccgtttactattgtcaacagtacggaagcagcccgtgga
cgtttggccagggcacgaaggtagaaatcaag
SEQ ID NO: 18
EIVLTQSPGTLSLSPGERATLSCRASQSVSSSYLAWYQQKPGQAPRLLIYGASSRATGIPDRFSGSGSGTDET
LTISRLEPEDFAVYYCQQYGSSPWTFGQGTKVEIK
SEQ ID NO: 19
cagtcagtctctagctcttat
SEQ ID NO: 20
QSVSSSY
SEQ ID NO: 21
ggggcaagt
SEQ ID NO: 22
GAS
SEQ ID NO: 23
caacagtacggaagcagcccgtggacg
SEQ ID NO: 24
QQYGSSPWT
SEQ ID NO: 25
caggttcagctggtgcagtctggagctgaggtgaagaagcctggggcctcagtgaaggtctcctgcaaggctt
ctggttacacctttaccagctatggtatcagctgggtgcgacaggcccctggacaaggacttgagtggatggg
atggatcagcccttacaatgttaacacaaactatgcacagaagctccagggcagagtcaccatgaccacagac
acatccacgaacacagcctacatggagctgaggagcctgagatctgacgactcggccgtgtattactgttcga
gagctgggatgccagctcgtcgctacggtatggacgtctggggccaagggaccacggtcaccgtctcctcagc
ctccaccaagggcccatcggtcttccccctggcgccctgctccaggagcacctccgagagcacagccgccctg
ggctgcctggtcaaggactacttccccgaaccggtgacggtgtcgtggaactcaggcgccctgaccagcggcg
tgcacaccttcccggctgtcctacagtcctcaggactctactccctcagcagcgtggtgaccgtgccctccag
cagcttgggcacgaagacctacacctgcaacgtagatcacaagcccagcaacaccaaggtggacaagagagtt
gagtccaaatatggtcccccatgcccaccgtgcccagcaccacctgtggcaggaccatcagtcttcctgttcc
ccccaaaacccaaggacactctcatgatctcccggacccctgaggtcacgtgcgtggtggtggacgtgagcca
ggaagaccccgaggtccagttcaactggtacgtggatggcgtggaggtgcataatgccaagacaaagccgcgg
gaggagcagttcaacagcacgtaccgtgtggtcagcgtcctcaccgtcctgcaccaggactggctgaacggca
aggagtacaagtgcaaggtctccaacaaaggcctcccgtcctccatcgagaaaaccatctccaaagccaaagg
gcagccccgagagccacaggtgtacaccctgcccccatcccaggaggagatgaccaagaaccaggtcagcctg
acctgcctggtcaaaggcttctaccccagcgacatcgccgtggagtgggagagcaatgggcagccggagaaca
actacaagaccacgcctcccgtgctggactccgacggctccttcttcctctacagcaggctcaccgtggacaa
gagcaggtggcaggaggggaatgtcttctcatgctccgtgatgcatgaggctctgcacaaccactacacacag
aagtccctctccctgtctctgggtaaatga
SEQ ID NO: 26
QVQLVQSGAEVKKPGASVKVSCKASGYTFTSYGISWVRQAPGQGLEWMGWISPYNVNTNYAQKLQGRVTMTTD
TSTNTAYMELRSLRSDDSAVYYCSRAGMPARRYGMDVWGQGTTVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSNTKVDKRV
ESKYGPPCPPCPAPPVAGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDKSRWQEGNVFSCSVMHEALHNHYTQ
KSLSLSLGK
SEQ ID NO: 27
caggtgcagctgcaggagtcgggcccaggactggtgaagccttcggagaccctgtccctcacctgcactgtct
ctggtggctccatcagtagttactactggagctggatccggcagcccccagggaagggactggagtggattgg
gtatatctattacagtgggatcacccactacaacccctccctcaagagtcgagtcaccatatcagtagacacg
tccaagatccagttctccctgaagctgagttctgtgaccgctgcggacacggccgtgtattactgtgcgagat
ggggggttcggagggactactactactacggtatggacgtctggggccaagggaccacggtcaccgtctcctc
agcctctacaaagggaccttctgtgtttcctctggctccttgttctagatctacatctgaatctacagctgct
ctgggatgtctggtgaaggattattttcctgaacctgtgacagtgtcttggaattctggagctctgacatctg
gagtgcatacatttcctgctgtgctgcagtcttctggactgtattctctgtcttctgtggtgacagtgccttc
ttcttctctgggaacaaagacatatacatgtaatgtggatcataagccttctaatacaaaggtggataagaga
gtggaatctaagtatggacctccttgtcctccttgtcctgctcctcctgtggctggaccttctgtgtttctgt
ttcctcctaagcctaaggatacactgatgatctctagaacacctgaagtgacatgtgtggtggtggatgtgtc
tcaggaagatcctgaagtgcagtttaattggtatgtggatggagtggaagtgcataatgctaagacaaagcct
agagaagaacagtttaattctacatatagagtggtgtctgtgctgacagtgctgcatcaggattggctgaatg
gaaaggaatataagtgtaaggtgtctaataagggactgccttcttctatcgaaaagacaatctctaaggctaa
gggacagcctagagaacctcaggtgtatacactgcctccttctcaggaagaaatgacaaagaatcaggtgtct
ctgacatgtctggtgaagggattttatccttctgatatcgctgtggaatgggaatctaatggacagcctgaaa
ataattataagacaacacctcctgtgctggattctgatggatctttttttctgtattctagactgacagtgga
taagtctagatggcaggaaggaaatgtgttttcttgttctgtgatgcatgaagctctgcataatagatttaca
cagaagtctctgtctctgtctcctggaaagtag
SEQ ID NO: 28
QVQLQESGPGLVKPSETLSLTCTVSGGSISSYYWSWIRQPPGKGLEWIGYIYYSGITHYNPSLKSRVTISVDT
SKIQFSLKLSSVTAADTAVYYCARWGVRRDYYYYGMDVWGQGTTVTVSSASTKGPSVFPLAPCSRSTSESTAA
LGCLVKDYFPEPVTVSWNSGALTSGVHTFPAVLQSSGLYSLSSVVTVPSSSLGTKTYTCNVDHKPSNTKVDKR
VESKYGPPCPPCPAPPVAGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVDKSRWQEGNVFSCSVMHEALHNRET
QKSLSLSPGK
SEQ ID NO: 29
gaaatagttttgacacagagtcccggcacactgtcactctctcccggggaaagagccaccttgtcatgtagag
caagtcagtcagtctctagctcttatctcgcctggtaccagcagaagccgggacaggcccctagactgctgat
ctacggggcaagttccagggccaccggaatccccgaccggttcagtggaagcggaagcggaaccgattttact
ttgacgatttctagactggagccagaggatttcgccgtttactattgtcaacagtacggaagcagcccgtgga
cgtttggccagggcacgaaggtagaaatcaagcgaactgtggctgcaccatctgtcttcatcttcccgccatc
tgatgagcagttgaaatctggaactgcctctgttgtgtgcctgctgaataacttctatcccagagaggccaaa
gtacagtggaaggtggataacgccctccaatcgggtaactcccaggagagtgtcacagagcaggacagcaagg
acagcacctacagcctcagcagcaccctgacgctgagcaaagcagactacgagaaacacaaagtctacgcctg
cgaagtcacccatcagggcctgagctcgcccgtcacaaagagcttcaacaggggagagtgttag
SEQ ID NO: 30
EIVLTQSPGTLSLSPGERATLSCRASQSVSSSYLAWYQQKPGQAPRLLIYGASSRATGIPDRFSGSGSGTDFT
LTISRLEPEDFAVYYCQQYGSSPWTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGEC
SEQ ID NO: 31
caggtgcagctgcaggagtcgggcccaggactggttcagccttcggagaccctgtccctcacctgcactgtct
ctggtggctccatcagtaattactactggagctggatccggcaggccccaggaaagggactggaatggattgg
atatgtctatcacagtgggagcaccaattccaagtccgccctcaagagtcgagtcaccatatcagttgacacg
tccaagaatcagttctcccttaaactgcggactgtgaccgccgcagacacggccgtatatttctgtgcgagac
atatcccagcccgttattactatggcatggacgtctggggccaagggaccacggtcaccgtctcctca
SEQ ID NO: 32
QVQLQESGPGLVQPSETLSLTCTVSGGSISNYYWSWIRQAPGKGLEWIGYVYHSGSTNSKSALKSRVTISVDT
SKNQFSLKLRTVTAADTAVYFCARHIPARYYYGMDVWGQGTTVTVSS
SEQ ID NO: 33
ggtggctccatcagtaattactac
SEQ ID NO: 34
GGSISNYY
SEQ ID NO: 35
gtctatcacagtgggagcacc
SEQ ID NO: 36
VYHSGST
SEQ ID NO: 37
gcgagacatatcccagcccgttattactatggcatggacgtc
SEQ ID NO: 38
ARHIPARYYYGMDV
SEQ ID NO: 39
caggtgcagctgcaggagtcgggcccaggactggttcagccttcggagaccctgtccctcacctgcactgtct
ctggtggctccatcagtaattactactggagctggatccggcaggccccaggaaagggactggaatggattgg
atatgtctatcacagtgggagcaccaattccaagtccgccctcaagagtcgagtcaccatatcagttgacacg
tccaagaatcagttctcccttaaactgcggactgtgaccgccgcagacacggccgtatatttctgtgcgagac
atatcccagcccgttattactatggcatggacgtctggggccaagggaccacggtcaccgtctcctcagcctc
caccaagggcccatcggtcttccccctggcgccctgctccaggagcacctccgagagcacagccgccctgggc
tgcctggtcaaggactacttccccgaaccggtgacggtgtcgtggaactcaggcgccctgaccagcggcgtgc
acaccttcccggctgtcctacagtcctcaggactctactccctcagcagcgtggtgaccgtgccctccagcag
cttgggcacgaagacctacacctgcaacgtagatcacaagcccagcaacaccaaggtggacaagagagttgag
tccaaatatggtcccccatgcccaccgtgcccagcaccacctgtggcaggaccatcagtcttcctgttccccc
caaaacccaaggacactctcatgatctcccggacccctgaggtcacgtgcgtggtggtggacgtgagccagga
agaccccgaggtccagttcaactggtacgtggatggcgtggaggtgcataatgccaagacaaagccgcgggag
gagcagttcaacagcacgtaccgtgtggtcagcgtcctcaccgtcctgcaccaggactggctgaacggcaagg
agtacaagtgcaaggtctccaacaaaggcctcccgtcctccatcgagaaaaccatctccaaagccaaagggca
gccccgagagccacaggtgtacaccctgcccccatcccaggaggagatgaccaagaaccaggtcagcctgacc
tgcctggtcaaaggcttctaccccagcgacatcgccgtggagtgggagagcaatgggcagccggagaacaact
acaagaccacgcctcccgtgctggactccgacggctccttcttcctctacagcaggctcaccgtggacaagag
caggtggcaggaggggaatgtcttctcatgctccgtgatgcatgaggctctgcacaaccactacacacagaag
tccctctccctgtctctgggtaaatga
SEQ ID NO: 40
QVQLQESGPGLVQPSETLSLTCTVSGGSISNYYWSWIRQAPGKGLEWIGYVYHSGSTNSKSALKSRVTISVDT
SKNQFSLKLRTVTAADTAVYFCARHIPARYYYGMDVWGQGTTVTVSSASTKGPSVFPLAPCSRSTSESTAALG
CLVKDYFPEPVTVSWNSGALTSGVHTFPAVLQSSGLYSLSSVVTVPSSSLGTKTYTCNVDHKPSNTKVDKRVE
SKYGPPCPPCPAPPVAGPSVFLFPPKPKDTLMISRTPEVTCVVVDVSQEDPEVQFNWYVDGVEVHNAKTKPRE
EQFNSTYRVVSVLTVLHQDWLNGKEYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKSRWQEGNVFSCSVMHEALHNHYTQK
SLSLSLGK
SEQ ID NO: 41
caggtgcagctgcaggagtcgggcccaggactggtgaagccttcggagaccctgtccctcacctgcactgtct
ctggtggctccatcagtagttactactggagttggatccggcagcccccagggaagggactggaatggattgg
gtctatttattacagtgggggcaccaattacaacccctccctcaagagtcgagtcaccatatcattagacacg
tccaagaaccagttctccctgaagctgagatctgggaccgctgcggacacggccgtttatttttgtaccagaa
ataactggttctacgggagggtggggggccttgactactggggccagggaaccctggtcaccgtctcctca
SEQ ID NO: 42
QVQLQESGPGLVKPSETLSLTCTVSGGSISSYYWSWIRQPPGKGLEWIGSIYYSGGTNYNPSLKSRVTISLDT
SKNQFSLKLRSGTAADTAVYFCTRNNWFYGRVGGLDYWGQGTLVTVSS
SEQ ID NO: 43
atttattacagtgggggcacc
SEQ ID NO: 44
IYYSGGT
SEQ ID NO: 45
accagaaataactggttctacgggagggtggggggccttgactac
SEQ ID NO: 46
TRNNWFYGRVGGLDY
SEQ ID NO: 47
caggtgcagctgcaggagtcgggcccaggactggtgaagccttcggagaccctgtccctcacctgcactgtct
ctggtggctccatcagtagttactactggagttggatccggcagcccccagggaagggactggaatggattgg
gtctatttattacagtgggggcaccaattacaacccctccctcaagagtcgagtcaccatatcattagacacg
tccaagaaccagttctccctgaagctgagatctgggaccgctgcggacacggccgtttatttttgtaccagaa
ataactggttctacgggagggtggggggccttgactactggggccagggaaccctggtcaccgtctcctcagc
ctccaccaagggcccatcggtcttccccctggcgccctgctccaggagcacctccgagagcacagccgccctg
ggctgcctggtcaaggactacttccccgaaccggtgacggtgtcgtggaactcaggcgccctgaccagcggcg
tgcacaccttcccggctgtcctacagtcctcaggactctactccctcagcagcgtggtgaccgtgccctccag
cagcttgggcacgaagacctacacctgcaacgtagatcacaagcccagcaacaccaaggtggacaagagagtt
gagtccaaatatggtcccccatgcccaccgtgcccagcaccacctgtggcaggaccatcagtcttcctgttcc
ccccaaaacccaaggacactctcatgatctcccggacccctgaggtcacgtgcgtggtggtggacgtgagcca
ggaagaccccgaggtccagttcaactggtacgtggatggcgtggaggtgcataatgccaagacaaagccgcgg
gaggagcagttcaacagcacgtaccgtgtggtcagcgtcctcaccgtcctgcaccaggactggctgaacggca
aggagtacaagtgcaaggtctccaacaaaggcctcccgtcctccatcgagaaaaccatctccaaagccaaagg
gcagccccgagagccacaggtgtacaccctgcccccatcccaggaggagatgaccaagaaccaggtcagcctg
acctgcctggtcaaaggcttctaccccagcgacatcgccgtggagtgggagagcaatgggcagccggagaaca
actacaagaccacgcctcccgtgctggactccgacggctccttcttcctctacagcaggctcaccgtggacaa
gagcaggtggcaggaggggaatgtcttctcatgctccgtgatgcatgaggctctgcacaaccactacacacag
aagtccctctccctgtctctgggtaaatga
SEQ ID NO: 48
QVQLQESGPGLVKPSETLSLTCTVSGGSISSYYWSWIRQPPGKGLEWIGSIYYSGGTNYNPSLKSRVTISLDT
SKNQFSLKLRSGTAADTAVYFCTRNNWFYGRVGGLDYWGQGTLVTVSSASTKGPSVEPLAPCSRSTSESTAAL
GCLVKDYFPEPVTVSWNSGALTSGVHTFPAVLQSSGLYSLSSVVTVPSSSLGTKTYTCNVDHKPSNTKVDKRV
ESKYGPPCPPCPAPPVAGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDKSRWQEGNVFSCSVMHEALHNHYTQ
KSLSLSLGK
SEQ ID NO: 49
NKILVKQSPMLVAYDNAVNLSCKYSYNLESREFRASLHKGLDSAVEVCVVYGNYSQQLQVYSKTGENCDGKLG
NESVTFYLQNLYVNQTDIYFCKIEVMYPPPYLDNEKSNGTIIHVKGKHLCPSPLFPGPSKPEPRGPTIKPCPP
CKCPAPNLLGGPSVFIFPPKIKDVLMISLSPIVTCVVVDVSEDDPDVQISWFVNNVEVHTAQTQTHREDYNST
LRVVSALPIQHQDWMSGKEFKCKVNNKDLPAPIERTISKPKGSVRAPQVYVLPPPEEEMTKKQVTLTCMVTDE
MPEDIYVEWTNNGKTELNYKNTEPVLDSDGSYFMYSKLRVEKKNWVERNSYSCSVVHEGLHNHHTTKSFSRTP
GK

The present disclosure is not to be limited in scope by the specific embodiments described herein. Indeed, various modifications of the disclosure in addition to those described herein will become apparent to those skilled in the art from the foregoing description. Such modifications are intended to fall within the scope of the appended claims.

1. An isolated bispecific antigen-binding molecule comprising:
(a) a first antigen-binding domain that specifically binds to human G protein-coupled receptor, class C group 5 member D (GPRC5D); and
(b) a second antigen-binding domain that specifically binds human CD28,
wherein the first antigen-binding domain comprises a heavy chain variable region (HCVR) comprising three heavy chain complementarity determining regions (CDRs), HCDR1, HCDR2 and HCDR3, and a light chain variable region (LCVR) comprising three light chain complementarity determining regions (CDRs), LCDR1, LCDR2 and LCDR3, wherein:
(i) HCDR1, HCDR2 and HCDR3 are the CDRs contained within a HCVR comprising the amino acid sequence of SEQ ID NO: 2; or
(ii) HCDR1, HCDR2 and HCDR3 are the CDRs contained within a HCVR comprising the amino acid sequence of SEQ ID NO: 32; or
(iii) HCDR1, HCDR2 and HCDR3 are the CDRs contained within a HCVR comprising the amino acid sequence of SEQ ID NO: 42, and
wherein LCDR1, LCDR2 and LCDR3 are the CDRs contained within a LCVR comprising the amino acid sequence of SEQ ID NO: 18.

2. The isolated bispecific antigen-binding molecule of claim 1, wherein the first antigen-binding domain binds to human GPRC5D within residues comprising an extracellular loop (ECL) 2 and ECL3 of GPRC5D.

3-4. (canceled)

5. The isolated bispecific antigen-binding molecule of claim 1, wherein the second antigen-binding domain comprises a heavy chain variable region (HCVR) comprising three heavy chain complementarity determining regions (CDRs), HCDR1, HCDR2 and HCDR3, and a light chain variable region (LCVR) comprising three light chain complementarity determining regions (CDRs), LCDR1, LCDR2 and LCDR3, wherein HCDR1, HCDR2 and HCDR3 are the CDRs contained within a HCVR comprising the amino acid sequence of SEQ ID NO: 10, and wherein LCDR1, LCDR2 and LCDR3 are the CDRs contained within a LCVR comprising the amino acid sequence of SEQ ID NO: 18.

6. (canceled)

7. The isolated bispecific antigen-binding molecule of claim 1, wherein:
(a) the first antigen-binding domain comprises a HCVR comprising HCDR1, HCDR2 and HCDR3 comprising the amino acid sequences of SEQ ID NOs: 4, 6 and 8, respectively, and a LCVR comprising LCDR1, LCDR2 and LCDR3 comprising the amino acid sequences of SEQ ID NOs: 20, 22 and 24, respectively; and
(b) the second antigen-binding domain comprises a HCVR comprising HCDR1, HCDR2 and HCDR3 comprising the amino acid sequences of SEQ ID NOs: 10, 12 and 14, respectively, and a LCVR comprising LCDR1, LCDR2 and LCDR3 comprising the amino acid sequences of SEQ ID NOs: 20, 22 and 24, respectively.

8. The isolated bispecific antigen-binding molecule of claim 7, wherein the first antigen-binding domain comprises a HCVR comprising the amino acid sequence of SEQ ID NO: 2 and a LCVR comprising the amino acid sequence of SEQ ID NO: 18, and the second antigen-binding domain comprises a HCVR comprising the amino acid sequence of SEQ ID NO: 10 and a LCVR comprising the amino acid sequence of SEQ ID NO: 18.

9. The isolated bispecific antigen-binding molecule of claim 8 that is a bispecific antibody comprising:
a first heavy chain and a paired light chain interconnected by disulfide bonds, wherein the first heavy chain comprises a HCVR and a heavy chain constant region comprising CH1, CH2, and CH3 domains, and the paired light chain comprises a LCVR and a light chain constant region, wherein the first heavy chain and paired light chain comprise the first antigen-binding domain; and
a second heavy chain and a paired light chain interconnected by disulfide bonds, wherein the second heavy chain comprises a HCVR and a heavy chain constant region comprising CH1, CH2, and CH3 domains, and the light chain comprises a LCVR and a light chain constant region, wherein the second heavy chain and paired light chain comprise the second antigen-binding domain.

10. The isolated bispecific antigen-binding molecule of claim 9, wherein:
(a) the first heavy chain or the second heavy chain, but not both, comprises a CH3 domain comprising a H435R (EU numbering) modification and a Y436F (EU numbering) modification;
(b) the heavy chain constant region of the first heavy chain and the heavy chain constant region of the second heavy chain are of isotype IgG1, or of isotype IgG4; and/or
(c) the first heavy chain and the second heavy chain comprise a chimeric hinge that reduces Fcγ receptor binding relative to a wild-type hinge of the same isotype.

11-13. (canceled)

14. The isolated bispecific antigen-binding molecule of claim 9, wherein the first heavy chain comprises the amino acid sequence of SEQ ID NO: 26, the second heavy chain comprises the amino acid sequence of SEQ ID NO: 28, and the paired light chain comprises the amino acid sequence of SEQ ID NO: 30.

15. The isolated bispecific antigen-binding molecule of claim 1, wherein:
(a) the first antigen-binding domain comprises a HCVR comprising HCDR1, HCDR2 and HCDR3 comprising the amino acid sequences of SEQ ID NOs: 12, 44 and 46, respectively, and a LCVR comprising LCDR1, LCDR2 and LCDR3 comprising the amino acid sequences of SEQ ID NOs: 20, 22 and 24, respectively; and
(b) the second antigen-binding domain comprises a HCVR comprising HCDR1, HCDR2 and HCDR3 comprising the amino acid sequences of SEQ ID NOs: 10, 12 and 14, respectively, and a LCVR comprising LCDR1, LCDR2 and LCDR3 comprising the amino acid sequences of SEQ ID NOs: 20, 22 and 24, respectively.

16. The isolated bispecific antigen-binding molecule of claim 15, wherein the first antigen-binding domain comprises a HCVR comprising the amino acid sequence of SEQ ID NO: 42 and a LCVR comprising the amino acid sequence of SEQ ID NO: 18, and the second antigen-binding domain comprises a HCVR comprising the amino acid sequence of SEQ ID NO: 10 and a LCVR comprising the amino acid sequence of SEQ ID NO: 18.

17. The isolated bispecific antigen-binding molecule of claim 16 that is a bispecific antibody comprising:
a first heavy chain and a paired light chain interconnected by disulfide bonds, wherein the first heavy chain comprises a HCVR and a heavy chain constant region comprising CH1, CH2, and CH3 domains, and the paired light chain comprises a LCVR and a light chain constant region, wherein the first heavy chain and paired light chain comprise the first antigen-binding domain; and
a second heavy chain and a paired light chain interconnected by disulfide bonds, wherein the second heavy chain comprises a HCVR and a heavy chain constant region comprising CH1, CH2, and CH3 domains, and the light chain comprises a LCVR and a light chain constant region, wherein the second heavy chain and paired light chain comprise the second antigen-binding domain.

18. The isolated bispecific antigen-binding molecule of claim 17, wherein:
(a) the first heavy chain or the second heavy chain, but not both, comprises a CH3 domain comprising a H435R (EU numbering) modification and a Y436F (EU numbering) modification;
(b) the heavy chain constant region of the first heavy chain and the heavy chain constant region of the second heavy chain are of isotype IgG1, or of isotype IgG4; and/or
(c) the first heavy chain and the second heavy chain comprise a chimeric hinge that reduces Fcγ receptor binding relative to a wild-type hinge of the same isotype.

19-21. (canceled)

22. The isolated bispecific antigen-binding molecule of claim 17, wherein the first heavy chain comprises the amino acid sequence of SEQ ID NO: 48, the second heavy chain comprises the amino acid sequence of SEQ ID NO: 28, and the paired light chain comprises the amino acid sequence of SEQ ID NO: 30.

23. The isolated bispecific antigen-binding molecule of claim 1, wherein:
(a) the first antigen-binding domain comprises a HCVR comprising HCDR1, HCDR2 and HCDR3 comprising the amino acid sequences of SEQ ID NOs: 34, 36 and 38, respectively, and a LCVR comprising LCDR1, LCDR2 and LCDR3 comprising the amino acid sequences of SEQ ID NOs: 20, 22 and 24, respectively; and
(b) the second antigen-binding domain comprises a HCVR comprising HCDR1, HCDR2 and HCDR3 comprising the amino acid sequences of SEQ ID NOs: 10, 12 and 14, respectively, and a LCVR comprising LCDR1, LCDR2 and LCDR3 comprising the amino acid sequences of SEQ ID NOs: 20, 22 and 24, respectively.

24. The isolated bispecific antigen-binding molecule of claim 23, wherein the first antigen-binding domain comprises a HCVR comprising the amino acid sequence of SEQ ID NO: 32 and a LCVR comprising the amino acid sequence of SEQ ID NO: 18, and the second antigen-binding domain comprises a HCVR comprising the amino acid sequence of SEQ ID NO: 10 and a LCVR comprising the amino acid sequence of SEQ ID NO: 18.

25. The isolated bispecific antigen-binding molecule of claim 24 that is a bispecific antibody comprising:
a first heavy chain and a paired light chain interconnected by disulfide bonds, wherein the first heavy chain comprises a HCVR and a heavy chain constant region comprising CH1, CH2, and CH3 domains, and the paired light chain comprises a LCVR and a light chain constant region, wherein the first heavy chain and paired light chain comprise the first antigen-binding domain; and
a second heavy chain and a paired light chain interconnected by disulfide bonds, wherein the second heavy chain comprises a HCVR and a heavy chain constant region comprising CH1, CH2, and CH3 domains, and the light chain comprises a LCVR and a light chain constant region, wherein the second heavy chain and paired light chain comprise the second antigen-binding domain.

26. The isolated bispecific antigen-binding molecule of claim 25, wherein:
(a) the first heavy chain or the second heavy chain, but not both, comprises a CH3 domain comprising a H435R (EU numbering) modification and a Y436F (EU numbering) modification;
(b) the heavy chain constant region of the first heavy chain and the heavy chain constant region of the second heavy chain are of isotype IgG1, or of isotype IgG4; and/or
(c) the first heavy chain and the second heavy chain comprise a chimeric hinge that reduces Fcγ receptor binding relative to a wild-type hinge of the same isotype.

27-29. (canceled)

30. The isolated bispecific antigen-binding molecule of claim 25, wherein the first heavy chain comprises the amino acid sequence of SEQ ID NO: 40, the second heavy chain comprises the amino acid sequence of SEQ ID NO: 28, and the paired light chain comprises the amino acid sequence of SEQ ID NO: 30.

31. A pharmaceutical composition comprising the isolated bispecific antigen-binding molecule of claim 1, and a pharmaceutically acceptable carrier or diluent.

32. A nucleic acid molecule comprising a nucleotide sequence encoding a bispecific antigen-binding molecule of claim 1, or a group of nucleic acid molecules comprising nucleotide sequences, respectively, encoding the HCVR of the first antigen-binding domain, the HCVR of the second antigen-binding domain, and the LCVR of the first and second antigen-binding domains of a bispecific antigen-binding molecule of claim 1.

33. An expression vector comprising the nucleic acid molecule of claim 32, or a group of expression vectors comprising, respectively, the group of nucleic acid molecules of claim 32.

34. A host cell comprising the expression vector or group of expression vectors of claim 33.

35. A host cell, comprising:
(a) an expression vector comprising a nucleic acid molecule encoding a first immunoglobulin heavy chain comprising the amino acid sequence of SEQ ID NO: 26;
(b) an expression vector comprising a nucleic acid molecule encoding a second immunoglobulin heavy chain comprising the amino acid sequence of SEQ ID NO: 28; and
(c) an expression vector comprising a nucleic acid molecule encoding an immunoglobulin light chain comprising the amino acid sequence of SEQ ID NO: 30; or
comprising:
(a) an expression vector comprising a nucleic acid molecule encoding a first immunoglobulin heavy chain comprising the amino acid sequence of SEQ ID NO: 48;
(b) an expression vector comprising a nucleic acid molecule encoding a second immunoglobulin heavy chain comprising the amino acid sequence of SEQ ID NO: 28; and
(c) an expression vector comprising a nucleic acid molecule encoding an immunoglobulin light chain comprising the amino acid sequence of SEQ ID NO: 30; or
comprising:
(a) an expression vector comprising a nucleic acid molecule encoding a first immunoglobulin heavy chain comprising the amino acid sequence of SEQ ID NO: 40;
(b) an expression vector comprising a nucleic acid molecule encoding a second immunoglobulin heavy chain comprising the amino acid sequence of SEQ ID NO: 28; and
(c) an expression vector comprising a nucleic acid molecule encoding an immunoglobulin light chain comprising the amino acid sequence of SEQ ID NO: 30.

36-37. (canceled)

38. A method of producing a bispecific antigen-binding molecule, comprising culturing the host cell of claim 34 under conditions permitting production of the bispecific antigen-binding molecule, and recovering the bispecific antigen-binding molecule so produced.

39. The method of claim 38, further comprising formulating the bispecific antigen-binding molecule as a pharmaceutical composition with a suitable carrier.

40. A method of treating a GPRC5D-expressing cancer in a subject in need thereof, the method comprising administering a bispecific antigen-binding molecule of claim 1, to the subject.

41. The method of claim 40, wherein:
(a) the GPRC5D-expressing cancer is multiple myeloma, non-small-cell lung cancer, head & neck cancer, breast cancer, colorectal cancer, pancreatic cancer, ovarian cancer, non-Hodgkin's lymphoma, renal cell carcinoma, lung cancer, liver cancer, or stomach cancer; and/or
(b) the subject has relapsed following prior therapy to treat the GPRC5D-expressing cancer, or the subject is refractory to at least one other therapy to treat the GPRC5D-expressing cancer.

42-43. (canceled)

44. A method of reducing risk of progression to symptomatic multiple myeloma in a subject in need thereof, comprising administering to the subject a bispecific antigen-binding molecule of claim 1, wherein the subject has been diagnosed with monoclonal gammopathy of undetermined significance (MGUS) or smoldering multiple myeloma (SMM), and wherein the risk of progression is reduced relative to a control population not administered the bispecific antigen-binding molecule.

45. The method of claim 44, wherein the subject is a subject that:
(a) has been diagnosed with MGUS;
(b) has been diagnosed with SMM;
(c) has a serum monoclonal protein level of <3 g/dL, clonal bone marrow plasma cells of <10%, and an absence of end-organ damage or a myeloma-defining event that can be attributed to the underlying plasma cell proliferative disorder; or
(d) exhibits one or more indicia of an enhanced risk of progression to symptomatic multiple myeloma.

46-49. (canceled)

50. The method of claim 44, further comprising identifying a subject that exhibits one or more indicia of an enhanced risk of progression to symptomatic multiple myeloma.

51. The method of claim 50, wherein the one or more indicia of an enhanced risk of progression to symptomatic multiple myeloma includes one or more of: (a) an M protein level of ≥5 g/L, ≥15 g/L, or ≥25 g/L; (b) presence of an IgM or IgA monoclonal protein; (c) presence of ≥5% clonal bone marrow plasma cells; and (d) an abnormal FLC ratio.

52. The method of claim 40, further comprising administering a second therapeutic agent or therapy.

53. The method of claim 52, wherein the second therapeutic agent or therapy comprises:
(a) a chemotherapeutic drug, DNA alkylators, immunomodulators, proteasome inhibitors, histone deacetylase inhibitors, radiotherapy, a stem cell transplant, a different bispecific antigen-binding molecule that interacts with a different tumor cell surface antigen and a T cell or immune cell antigen, an antibody drug conjugate, a bispecific antibody that specifically binds human B-cell maturation antigen (BCMA) and human CD3, a PD-1 inhibitor, PD-L1 inhibitor, CTLA-4 inhibitor, LAG-3 inhibitor, or combinations thereof;
(b) a bispecific antibody that specifically binds human BCMA and human CD3; or
(c) linvoseltamab.

54-55. (canceled)

56. An isolated antibody or antigen binding fragment thereof that specifically binds to human G protein-coupled receptor, class C group 5 member D (GPRC5D), wherein the antibody or antigen-binding fragment thereof comprises a heavy chain variable region (HCVR) comprising three heavy chain complementarity determining regions HCDR1, HCDR2 and HCDR3, and a light chain variable region (LCVR) comprising three light chain complementarity determining regions, LCDR1, LCDR2 and LCDR3, wherein the HCDR1, HCDR2 and HCDR3 are the complementarity determining regions contained within a HCVR comprising the amino acid sequence of SEQ ID NO: 2, 32 or 42, and the LCDR1, LCDR2 and LCDR3 are the complementarity determining regions contained within a LCVR comprising the amino acid sequence of SEQ ID NO: 18.

57. The isolated antibody or antigen-binding fragment of claim 56, wherein HCDR1, HCDR2, HCDR3, LCDR1, LCDR2, and LCDR3 comprise the amino acid sequences of SEQ ID NOs: 4, 6, 8, 20, 22, and 24, respectively.

58. The isolated antibody or antigen-binding fragment of claim 57, comprising a HCVR comprising the amino acid sequence of SEQ ID NO: 2 and a LCVR comprising the amino acid sequence of SEQ ID NO: 18.

59. The isolated antibody or antigen-binding fragment of claim 58, comprising a heavy chain comprising the amino acid sequence of SEQ ID NO: 26 and a light chain comprising the amino acid sequence of SEQ ID NO: 30.

60. The isolated antibody or antigen-binding fragment of claim 56, wherein HCDR1, HCDR2, HCDR3, LCDR1, LCDR2, and LCDR3 comprise the amino acid sequences of SEQ ID NOs: 34, 36, 38, 20, 22, and 24, respectively.

61. The isolated antibody or antigen-binding fragment of claim 60, comprising a HCVR comprising the amino acid sequence of SEQ ID NO: 32 and a LCVR comprising the amino acid sequence of SEQ ID NO: 18.

62. The isolated antibody or antigen-binding fragment of claim 61, comprising a heavy chain comprising the amino acid sequence of SEQ ID NO: 40 and a light chain comprising the amino acid sequence of SEQ ID NO: 30.

63. The isolated antibody or antigen-binding fragment of claim 56, wherein HCDR1, HCDR2, HCDR3, LCDR1, LCDR2, and LCDR3 comprise the amino acid sequences of SEQ ID NOs: 12, 44, 46, 20, 22, and 24, respectively.

64. The isolated antibody or antigen-binding fragment of claim 63, comprising a HCVR comprising the amino acid sequence of SEQ ID NO: 42 and a LCVR comprising the amino acid sequence of SEQ ID NO: 18.

65. The isolated antibody or antigen-binding fragment of claim 64, comprising a heavy chain comprising the amino acid sequence of SEQ ID NO: 48 and a light chain comprising the amino acid sequence of SEQ ID NO: 30.

66. A pharmaceutical composition comprising the isolated antibody or antigen-binding fragment of claim 56, and a pharmaceutically acceptable carrier or diluent.

67. A nucleic acid molecule comprising a nucleotide sequence encoding an antibody or antigen-binding fragment of claim 56, or a pair of nucleic acid molecules comprising nucleotide sequences, respectively, encoding the HCVR and the LCVR of the antibody or antigen-binding fragment of claim 56.

68. An expression vector comprising the nucleic acid molecule of claim 67, or a pair of expression vectors comprising, respectively, the pair of nucleic acid molecules of claim 67.

69. A host cell comprising the expression vector or pair of expression vectors of claim 68.

70. A method of treating a GPRC5D-expressing cancer in a subject in need thereof, the method comprising administering an antibody or antigen-binding fragment of claim 56 to the subject, optionally wherein the GPRC5D-expressing cancer is multiple myeloma.
